(19) United States
(12) Patent Application Publication
(10) Pub. No.: US 2021/0206758 A1
(43) Pub. Date: Jul. 8, 2021

(54) NAPHTHYRIDINES AS INTEGRIN ANTAGONISTS

(71) Applicant: GlaxoSmithKline Intellectual Property Development Limited, Brentford, Middlesex (GB)

(72) Inventors: Niall Andrew ANDERSON, Stevenage (GB); Matthew Howard James CAMPBELL-CRAWFORD, Stevenage (GB); Ashley Paul HANCOCK, Stevenage (GB); Seble LEMMA, Stevenage (GB); John Martin PRITCHARD, Stevenage (GB); Panayiotis Alexandrou PROCOPIOU, Stevenage (GB); Joanna Mary REDMOND, Stevenage (GB); Steven Leslie SOLLIS, Stevenage (GB)

(21) Appl. No.: 16/087,349

(22) PCT Filed: Mar. 20, 2017
(86) PCT No.: PCT/EP2017/056527

§ 371 Date: Sep. 21, 2018

(30) Foreign Application Priority Data
Mar. 21, 2016 (GB) 1604681.5

(51) Int. Cl. C07D 471/04

(52) U.S. Cl. CPC C07D 471/04; C07B 2200/13

(57) ABSTRACT

The invention relates to compounds of Formula (I):

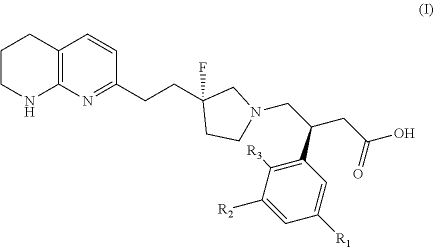

wherein R₁, R₂ and R₃ are as defined in the description and claims, or pharmaceutically acceptable salts thereof, having αᵥβ₆ integrin antagonist activity. The invention also relates to pharmaceutical compositions including a compound of Formula (I), or a pharmaceutically acceptable salt thereof, and to the use of a compound of Formula (I), or a pharmaceutically acceptable salt thereof, in therapy, including in the treatment of a disease or condition for which an αᵥβ₆ integrin antagonist is indicated, in particular the treatment of idiopathic pulmonary fibrosis.

### FIELD OF THE INVENTION

The present invention relates to pyrrolidine compounds being αᵥβ₆ integrin antagonists, pharmaceutical compositions comprising such compounds and to their use in therapy, especially in the treatment of conditions for which an αᵥβ₆ integrin antagonist is indicated, to the use of a compound in the manufacture of a medicament for the treatment of conditions in which an antagonist of αᵥβ₆ integrin is indicated, and a method for the treatment of disorders in which antagonism of αᵥβ₆ integrin is indicated in a human.

### BACKGROUND OF THE INVENTION

Integrin superfamily proteins are heterodimeric cell surface receptors, composed of an alpha and beta subunit. At least 18 alpha and 8 beta subunits have been reported, which have been demonstrated to form 24 distinct alpha/beta heterodimers. Each chain comprises a large extracellular domain (>640 amino acids for the beta subunit, >940 amino acids for the alpha subunit), with a transmembrane spanning region of around 20 amino acids per chain, and generally a short cytoplasmic tail of 30-50 amino acids per chain. Different integrins have been shown to participate in a plethora of cellular biologies, including cell adhesion to the extracellular matrix, cell-cell interactions, and effects on call migration, proliferation, differentiation and survival (Barczyk et al, Cell and Tissue Research, 2010, 339, 269).

Integrin receptors interact with binding proteins via short protein-protein binding interfaces.

The integrin family can be grouped into sub-families that share similar binding recognition motifs in such ligands. A major subfamily is the RGD-integrins, which recognise ligands that contain an RGD (Arginine-glycine-aspartic acid) motif within their protein sequence. There are 8 integrins in this subfamily, namely αᵥβ₁, αᵥβ₃, αᵥβ₅, αᵥβ₆, αᵥβ₈, α_{IIb}β₃, α₅β₁, α₈β₁, where nomenclature demonstrates that αᵥβ₁, αᵥβ₃, αᵥβ₅, αᵥβ₆, & αᵥβ₈ share a common αᵥ subunit with a divergent β subunit, and αᵥβ₁, α₅β₁ & α₈β₁ share a common β₁ subunit with a divergent a subunit. The β₁ subunit has been shown to pair with 11 different a subunits, of which only the 3 listed above commonly recognise the RGD peptide motif. (Humphries et al, Journal of Cell Science, 2006, 119, 3901).

The 8 RGD-binding integrins have different binding affinities and specificities for different RGD-containing ligands. Ligands include proteins such as fibronectin, vitronectin, osteopontin, and the latency associated peptides (LAPs) of Transforming Growth Factor β₁ and β₃ (TGFβ₁ and TGFβ₃). Integrin binding to the LAPs of TGFβ₁ and TGFβ₃ has been shown in several systems to enable activation of the TGFβ₁ and TGFβ₃ biological activities, and subsequent TGFβ-driven biologies (Worthington et al, Trends in Biochemical Sciences 2011, 36, 47). The diversity of such ligands, coupled with expression patterns of RGD-binding integrins, generates multiple opportunities for disease intervention. Such diseases include fibrotic diseases (Margadant et al, EMBO reports, 2010, 11, 97), inflammatory disorders, cancer (Desgrosellier et al, Nature Reviews Cancer, 2010, 10, 9), restenosis, and other diseases with an angiogenic component (Weis et al, Cold Spring. Harb. Perspect. Med. 2011, 1, a 006478).

A significant number of αᵥ integrin inhibitors (Goodman et al, Trends in Pharmacological Sciences, 2012, 33, 405) have been disclosed in the literature including inhibitory antibodies, peptides and small molecules. For antibodies these include the pan-αᵥ inhibitor Intetumumab, the selective αᵥβ₃ inhibitor Etaraczumab, and the selective αᵥβ₆ inhibitor STX-100. Cilengitide is a cyclic peptide inhibitor that inhibits both αᵥβ₃ and αᵥβ₅, and SB-267268 is an example of a compound (Wilkinson-Berka et al, Invest. Ophthalmol. Vis Sci., 2006, 47, 1600), which inhibits both αᵥβ₃ and αᵥβ₅. Invention of compounds to act as inhibitors of differing combinations of αᵥ integrins enables novel agents to be generated tailored for specific disease indications.

Pulmonary fibrosis represents the end stage of several interstitial lung diseases, including the idiopathic interstitial pneumonias, and is characterised by the excessive deposition of extracellular matrix within the pulmonary interstitium. Among the idiopathic interstitial pneumonias, idiopathic pulmonary fibrosis (IPF) represents the commonest and most fatal condition with a typical survival of 3 to 5 years following diagnosis. Fibrosis in IPF is generally progressive, refractory to current pharmacological intervention and inexorably leads to respiratory failure due to obliteration of functional alveolar units. IPF affects approximately 500,000 people in the USA and Europe.

There are in vitro experimental animal and IPF patient immunohistochemistry data to support a key role for the epithelially restricted integrin, αᵥβ₆, in the activation of TGFβ1. Expression of this integrin is low in normal epithelial tissues and is significantly up-regulated in injured and inflamed epithelia including the activated epithelium in IPF. Targeting this integrin, therefore, reduces the theoretical possibility of interfering with wider TGFβ homeostatic roles. Partial inhibition of the αᵥβ₆ integrin by antibody blockade has been shown to prevent pulmonary fibrosis without exacerbating inflammation (Horan G S et al Partial inhibition of integrin αᵥβ₆ prevents pulmonary fibrosis without exacerbating inflammation. Am J Respir Crit Care Med 2008 177: 56-65). Outside of pulmonary fibrosis, αᵥβ₆ is also considered an important promoter of fibrotic disease of other organs, including liver and kidney (Reviewed in Henderson N C et al Integrin-mediated regulation of TGFβ in Fibrosis, Biochimica et Biophysica Acta—Molecular Basis of Disease 2013 1832:891-896), suggesting that an αᵥβ₆ inhibitor could be effective in treating fibrotic diseases in multiple organs.

Consistent with the observation that several RGD-binding integrins can bind to, and activate, TGFβ, different αᵥ integrins have recently been implicated in fibrotic disease (Henderson N C et al Targeting of αᵥ integrin identifies a core molecular pathway that regulates fibrosis in several organs Henderson N C et al Targeting of αᵥ integrin identifies a core molecular pathway that regulates fibrosis in several organs Nature Medicine 2013 19: 1617-1627). Therefore inhibitors against specific members of the RGD binding integrin families, or with specific selectivity fingerprints within the RGD binding integrin family, may be effective in treating fibrotic diseases in multiple organs.

WO 2016/046225 A1 (published 31 Mar. 2016) disclosed compounds of formula

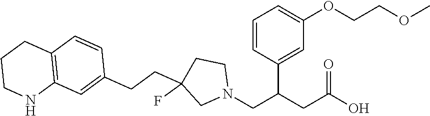 [SMILES: COCCOc1cccc(C(CC(=O)O)CN2CCC(F)(CCc3ccc4c(c3)NCCC4)C2)c1]

and salts thereof as αᵥβ₆ antagonists, including the specific diastereoisomer (S)-4-((S)-3-Fluoro-3-(2-(5,6,7,8-tetrahydro-1,8-naphthyridin-2-yl)ethyl)pyrrolidin-1-yl)-3-(3-(2-methoxyethoxy)phenyl) butanoic acid and a maleate and a citraconate salt thereof.

It is an object of the invention to provide αᵥβ₆ antagonists, including those with activities against other αᵥ integrins, such as αᵥβ₁, αᵥβ₃, αᵥβ₅ or αᵥβ₈.

### BRIEF SUMMARY OF THE INVENTION

In a first aspect of the present invention, there is provided a compound of Formula (I):

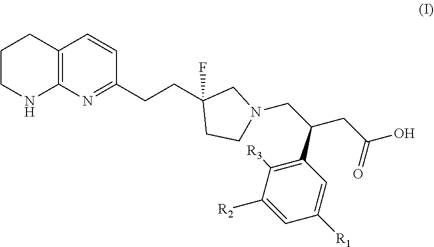

wherein
either R₁ and R₂ each independently represent hydrogen or a group —O—CR₅R₆—CR₇R₈—O(C₁₋₂-alkyl)
   wherein R₅, R₆, R₇ and R₈ each independently represent hydrogen or methyl;
   with the proviso that R₁ and R₂ cannot both represent hydrogen;
or R₂ represents hydrogen and R₁ represents
   (i) a group selected from

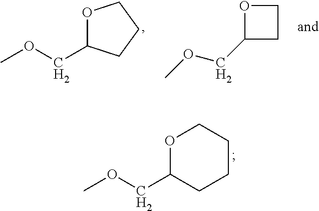

or
(ii) a group selected from

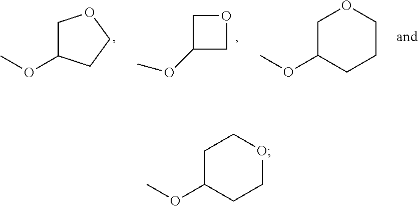

or
(iii) a group selected from

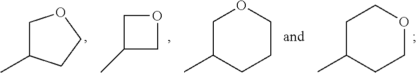

or R₂ represents hydrogen and R₁ represents

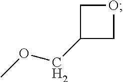

or one of R₁ and R₂ represents a group —O(CH₂)₂OMe and the other represents —O(CH₂)₂F;
and R₃ represents hydrogen or fluoro; with the proviso that where R₁ and R₂ both represent other than hydrogen then R₃ represents hydrogen;
provided that the compound is not (S)-4-((S)-3-fluoro-3-(2-(5,6,7,8-tetrahydro-1,8-naphthyridin-2-yl)ethyl)pyrrolidin-1-yl)-3-(3-(2-methoxyethoxy)phenyl)butanoic acid;
   or a pharmaceutically acceptable salt thereof.

Compounds of Formula (I) and their pharmaceutically acceptable salts have αᵥβ₆ integrin antagonist activity and are believed to be of potential use for the treatment of certain disorders. The term αᵥβ₆ antagonist activity includes αᵥβ₆ inhibitor activity herein.

In a second aspect of the present invention, there is provided a pharmaceutical composition comprising a compound of Formula (I) or a pharmaceutically acceptable salt thereof and a pharmaceutically acceptable carrier, diluent or excipient.

In a third aspect of the present invention, there is provided a compound of Formula (I), or a pharmaceutically acceptable salt thereof for use in therapy, in particular in the treatment of a disease or condition for which an αᵥβ₆ integrin antagonist is indicated.

In a fourth aspect of the present invention, there is provided a method of treatment of a disease or condition for which an αᵥβ₆ integrin antagonist is indicated in a human in need thereof which comprises administering to such human a therapeutically effective amount of compound of Formula (I) or a pharmaceutically acceptable salt thereof.

In a fifth aspect of the present invention, there is provided the use of a compound of Formula (I) or a pharmaceutically acceptable salt thereof in the manufacture of a medicament for the treatment of a disease or condition for which an αᵥβ₆ integrin antagonist is indicated.

### BRIEF DESCRIPTION OF THE DRAWINGS

FIG. 1 illustrates the X-ray crystal structure of the intermediate compound of Formula (XX).

### DETAILED DESCRIPTION OF THE INVENTION

The present invention relates to a compound of Formula (I):

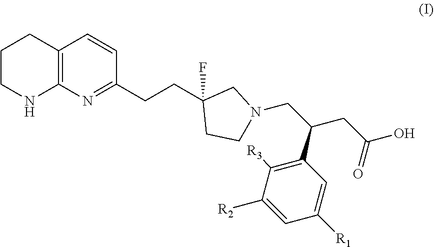

wherein
either R₁ and R₂ each independently represent hydrogen or a group —O—CR₅R₆—CR₇R₈—O(C₁₋₂-alkyl)
   wherein R₅, R₆, R₇ and R₈ each independently represent hydrogen or methyl;
   with the proviso that R₁ and R₂ cannot both represent hydrogen;
      or R₂ represents hydrogen and R₁ represents
(i) a group selected from

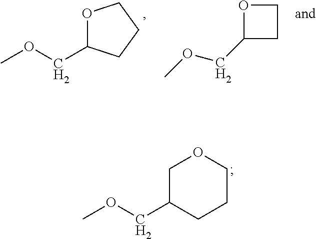

or
(ii) a group selected from

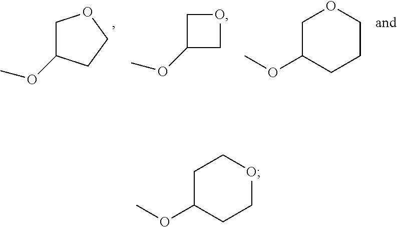

or
(iii) a group selected from

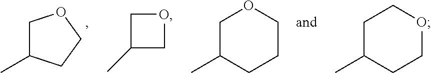

or R₂ represents hydrogen and R₁ represents

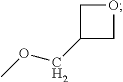

or one of R₁ and R₂ represents a group —O(CH₂)₂OMe and the other represents —O(CH₂)₂F;
and R₃ represents hydrogen or fluoro; but wherein when R₁ and R₂ both represent other than hydrogen then R₃ represents hydrogen;
provided that the compound is not (S)-4-((S)-3-fluoro-3-(2-(5,6,7,8-tetrahydro-1,8-naphthyridin-2-yl)ethyl)pyrrolidin-1-yl)-3-(3-(2-methoxyethoxy)phenyl)butanoic acid;
   or a pharmaceutically acceptable salt thereof.

In an embodiment the present invention relates to a compound of Formula (I) wherein either R₁ and R₂ each independently represent hydrogen or a group —O—CR₅R₆—CR₇R₈—O(C₁₋₂-alkyl)
wherein R₅, R₆, R₇ and R₈ each independently represent hydrogen or methyl;
with the proviso that R₁ and R₂ cannot both represent hydrogen;
   or R₂ represents hydrogen and R₁ represents
(i) a group selected from

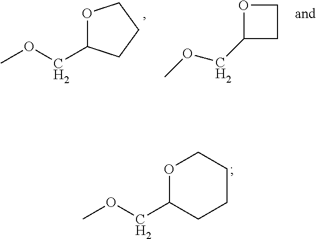

or
(ii) a group selected from

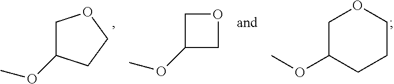

or
(iii) a group selected from

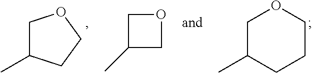

or R₂ represents hydrogen and R₁ represents

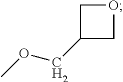

or one of R₁ and R₂ represents a group —O(CH₂)₂OMe and the other represents —O(CH₂)₂F;
and R₃ represents hydrogen or fluoro; but wherein when R₁ and R₂ both represent other than hydrogen then R₃ represents hydrogen;
provided that the compound is not (S)-4-((S)-3-fluoro-3-(2-(5,6,7,8-tetrahydro-1,8-naphthyridin-2-yl)ethyl)pyrrolidin-1-yl)-3-(3-(2-methoxyethoxy)phenyl)butanoic acid;
   or a pharmaceutically acceptable salt thereof.

In an embodiment, R₁ and R₂ each independently represent hydrogen or a group —O—CR₅R₆—CR₇R₈—O(C₁₋₂-alkyl) wherein R₅, R₆, R₇ and R₈ each independently represent hydrogen or methyl; with the proviso that R₁ and R₂ cannot both represent hydrogen.

In an embodiment, one of R₁ and R₂ represents hydrogen and the other represents a group —O—CR₅R₆—CR₇R₈—O(C₁₋₂-alkyl) wherein R₅, R₆, R₇ and R₈ each independently represent hydrogen or methyl.

In an embodiment, both of R₁ and R₂ represents a group —O—CR₅R₆—CR₇R₈—O(C₁₋₂-alkyl) wherein R₅, R₆, R₇ and R₈ each independently represent hydrogen or methyl.

In an embodiment, one of R₁ and R₂ represents hydrogen and the other represents a group selected from 2-methoxyethoxy, 2-methoxypropoxy, 2-methoxy-2-methylpropoxy, (1-methoxypropan-2-yl)oxy, or (1-methoxy-2-methylpropan-2-yl)oxy. In a further embodiment, one of R₁ and R₂ represents hydrogen and the other represents a group selected from 2-methoxypropoxy or (1-methoxy-2-methylpropan-2-yl)oxy.

In a specific embodiment, both of R₁ and R₂ represent 2-methoxyethoxy.

In an embodiment R₂ represents hydrogen and R₁ represents a group selected from

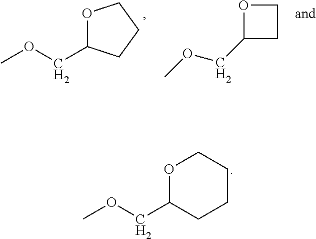

In a specific embodiment R₂ represents hydrogen and R₁ represents (tetrahydrofuran-2-yl)methoxy.

In an embodiment R₂ represents hydrogen and R₁ represents a group selected from

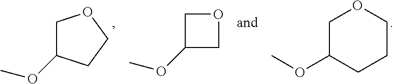

In a specific embodiment R₂ represents hydrogen and R₁ represents a group

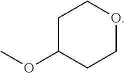

In a specific embodiment R₂ represents hydrogen and R₁ represents (tetrahydrofuran-3-yl)oxy.

In a specific embodiment R₂ represents hydrogen and R₁ represents (oxetan-3-yl)oxy.

In an embodiment R₂ represents hydrogen and R₁ represents a group selected from

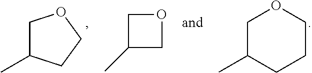

In a specific embodiment R₂ represents hydrogen and R₁ represents tetrahydrofuran-3-yl.

In a specific embodiment R₂ represents hydrogen and R₁ represents oxetan-3-yl.

In a specific embodiment R₃ represents hydrogen. In a further specific embodiment R₃ represents fluoro.

In an embodiment, R₃ represents fluoro, R₂ represents hydrogen; and R₁ is as defined above.

It is to be understood that the present invention covers all combinations of particular groups described hereinabove.

In an embodiment, specific compounds of this invention include:
(S)-4-((S)-3-Fluoro-3-(2-(5,6,7,8-tetrahydro-1,8-naphthyridin-2-yl)ethyl)pyrrolidin-1-yl)-3-(3-(((R)-tetrahydrofuran-2-yl)methoxy)phenyl)butanoic acid; or
(S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-2-yl)methoxy)phenyl)butanoic acid;
   or a pharmaceutically acceptable salt thereof.

In a further embodiment, specific compounds of this invention include:
(S)-4-((S)-3-Fluoro-3-(2-(5,6,7,8-tetrahydro-1,8-naphthyridin-2-yl)ethyl)pyrrolidin-1-yl)-3-(3-(((R)-tetrahydrofuran-3-yl)oxy)phenyl)butanoic acid;
(S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoic acid; or
(S)-4-((S)-3-Fluoro-3-(2-(5,6,7,8-tetrahydro-1,8-naphthyridin-2-yl)ethyl)pyrrolidin-1-yl)-3-(3-(oxetan-3-yloxy)phenyl)butanoic acid;
   or a pharmaceutically acceptable salt thereof.

In a further embodiment, specific compounds of this invention include:
(3S)-4-((S)-3-Fluoro-3-(2-(5,6,7,8-tetrahydro-1,8-naphthyridin-2-yl)ethyl)pyrrolidin-1-yl)-3-(3-(tetrahydrofuran-3-yl)phenyl)butanoic acid (Isomer 1);
(3S)-4-((S)-3-Fluoro-3-(2-(5,6,7,8-tetrahydro-1,8-naphthyridin-2-yl)ethyl)pyrrolidin-1-yl)-3-(3-(tetrahydrofuran-3-yl)phenyl)butanoic acid (Isomer 2);
   or a pharmaceutically acceptable salt thereof.

In a further embodiment, specific compounds of this invention include:
((S)-4-((S)-3-Fluoro-3-(2-(5,6,7,8-tetrahydro-1,8-naphthyridin-2-yl)ethyl)pyrrolidin-1-yl)-3-(3-((R)-2-methoxypropoxy)phenyl)butanoic acid;
((S)-4-((S)-3-Fluoro-3-(2-(5,6,7,8-tetrahydro-1,8-naphthyridin-2-yl)ethyl)pyrrolidin-1-yl)-3-(3-((S)-2-methoxypropoxy)phenyl)butanoic acid;
(S)-4-((S)-3-Fluoro-3-(2-(5,6,7,8-tetrahydro-1,8-naphthyridin-2-yl)ethyl)pyrrolidin-1-yl)-3-(3-((1-methoxy-2-methylpropan-2-yl)oxy)phenyl)butanoic acid;
(S)-3-(3,5-Bis(2-methoxyethoxy)phenyl)-4-((S)-3-fluoro-3-(2-(5,6,7,8-tetrahydro-1,8-naphthyridin-2-yl)ethyl)pyrrolidin-1-yl)butanoic acid;
   or a pharmaceutically acceptable salt thereof.

In a further embodiment, specific compounds of this invention include:
(S)-4-((S)-3-Fluoro-3-(2-(5,6,7,8-tetrahydro-1,8-naphthyridin-2-yl)ethyl)pyrrolidin-1-yl)-3-(3-(oxetan-3-ylmethoxy)phenyl)butanoic acid;
4-((S)-3-Fluoro-3-(2-(5,6,7,8-tetrahydro-1,8-naphthyridin-2-yl)ethyl)pyrrolidin-1-yl)-3-(3-(2-fluoroethoxy)-5-(2-methoxyethoxy)phenyl)butanoic acid;
4-((S)-3-Fluoro-3-(2-(5,6,7,8-tetrahydro-1,8-naphthyridin-2-yl)ethyl)pyrrolidin-1-yl)-3-(2-fluoro-5-(2-methoxyethoxy)phenyl)butanoic acid;
   or a pharmaceutically acceptable salt thereof.

In a further embodiment, specific compounds of this invention include:
(S)-4-((S)-3-Fluoro-3-(2-(5,6,7,8-tetrahydro-1,8-naphthyridin-2-yl)ethyl)pyrrolidin-1-yl)-3-(3-((tetrahydro-2H-pyran-4-yl)oxy)phenyl)butanoic acid;
   or a pharmaceutically acceptable salt thereof.

In a further embodiment, specific compounds of this invention include:
(S)-4-((S)-3-Fluoro-3-(2-(5,6,7,8-tetrahydro-1,8-naphthyridin-2-yl)ethyl)pyrrolidin-1-yl)-3-(3-(((3-tetrahydrofuran-3-yl)oxy)phenyl)butanoic acid citrate salt; or
(S)-4-((S)-3-Fluoro-3-(2-(5,6,7,8-tetrahydro-1,8-naphthyridin-2-yl)ethyl)pyrrolidin-1-yl)-3-(3-(((3-tetrahydrofuran-3-yl)oxy)phenyl)butanoic acid maleate salt.

In a further embodiment, specific compounds of this invention include: (S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoic acid citrate salt; or
(S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoic acid maleate salt.

Compounds of Formula (I) have both a basic amine group and a carboxylic acid group and can consequently be in the form of a zwitterion, also known as an inner salt. Therefore, in an embodiment the compound of Formula (I) is in a zwitterion form.

It will be appreciated that the present invention covers compounds of Formula (I) as the parent compound and as pharmaceutically acceptable salts thereof. In one embodiment the invention relates to compounds of Formula (I). In another embodiment the invention relates to a pharmaceutically acceptable salt of a compound of Formula (I).

As used herein, the term ‘pharmaceutically acceptable salt’ refers to a salt that retains the desired biological activity of the subject compound and exhibits minimal undesired toxicological effects.

For a review of suitable pharmaceutically acceptable salts see Berge et al., J. Pharm. Sci., 66: 1-19, (1977). Suitable pharmaceutically acceptable salts are also listed in P H Stahl and C G Wermuth, editors, Handbook of Pharmaceutical Salts; Properties, Selection and Use, Weinheim/Zurich: Wiley-VCH/VHCA, 2002.

Suitable pharmaceutically acceptable salts can include acid addition salts with inorganic acids such, for example, as hydrochloric acid, hydrobromic acid, nitric acid, phosphoric acid, or sulfuric acid, or with organic acids such, for example as methanesulfonic acid, ethanesulfonic acid, p-toluenesulfonic acid, acetic acid, propionic acid, lactic acid, citric acid, fumaric acid, malic acid, succinic acid, salicylic acid, maleic acid, glycerophosphoric acid, tartaric, benzoic, glutamic, aspartic, benzenesulfonic, naphthalenesulfonic such as 2-naphthalenesulfonic, hexanoic acid or acetylsalicylic acid. Suitable pharmaceutically acceptable salts can include base addition salts such as, for example, ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

In an embodiment the pharmaceutically acceptable salt is a maleate salt or a citrate salt.

Typically, a pharmaceutically acceptable salt may readily be prepared by reaction with the appropriate acid or base, optionally in a suitable solvent such as an organic solvent. The resultant salt may be isolated by crystallisation and filtration or may be recovered by evaporation of the solvent.

Other non-pharmaceutically acceptable salts, e.g. formates, oxalates or trifluoroacetates, may be used, for example, in the preparation of the compounds of Formula (I) and their pharmaceutically acceptable salts.

The invention includes within its scope all possible stoichiometric and non-stoichiometric forms of the pharmaceutically acceptable salts of the compounds of Formula (I).

It will be appreciated that many organic compounds can form complexes with solvents in which they are reacted or from which they are precipitated or crystallized. These complexes are known as “solvates”. For example, a complex with water is known as a “hydrate”. Solvents with high boiling points and/or capable of forming hydrogen bonds such as water, xylene, N-methyl pyrrolidinone, methanol and ethanol may be used to form solvates. Methods for identification of solvates include, but are not limited to, NMR and microanalysis. The compounds of Formula (I) and pharmaceutically acceptable salts thereof may exist in solvated and unsolvated form.

The compounds of Formula (I) may be in crystalline or amorphous form. Furthermore, some of the crystalline forms of the compounds of Formula (I) may exist in different polymorphic forms. Polymorphic forms of compounds of Formula (I) may be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (SSNMR).

The compounds of Formula (I) may contain one or more asymmetric centres as a result of the groups R₁ and R₂ as defined above, so that optical isomers, e.g. diastereoisomers may be formed. Accordingly, the present invention encompasses such isomers of the compounds of Formula (I) whether as individual isomers isolated such as to be substantially free of the other isomer (i.e. pure) or as mixtures. An individual isomer isolated such as to be substantially free of the other isomer (i.e. pure) may be isolated such that less than 10%, particularly less than about 1%, for example less than about 0.1% of the other isomer is present.

Separation of isomers may be achieved by conventional techniques known to those skilled in the art, e.g. by fractional crystallisation, chromatography, HPLC or a combination of these techniques.

Compounds of Formula (I) may exist in one of several tautomeric forms. It will be understood that the present invention encompasses all tautomers of the compounds of Formula (I) whether as individual tautomers or as mixtures thereof.

### Definitions

Terms are used within their accepted meanings. The following definitions are meant to clarify, but not limit, the terms defined.

As used herein, the term “alkyl” represents a saturated, straight or branched hydrocarbon moiety having the specified number of carbon atoms. The term “(C₁-C₂)alkyl” in the definition of R₁ and R₂ above refers to an unsubstituted alkyl moiety containing 1 or 2 carbon atoms; exemplary alkyls include methyl and ethyl. In an embodiment the term “(C₁-C₂)alkyl” in the definition of R₁ and R₂ above represents methyl. In an embodiment the term “(C₁-C₂)alkyl” in the definition of R₁ and R₂ above represents ethyl.

As used herein, the term “optionally” means that the subsequently described event(s) may or may not occur, and includes both event(s) that occur and event(s) that do not occur.

As used herein, the term “treatment” refers to alleviating the specified condition, eliminating or reducing one or more symptoms of the condition, slowing or eliminating the progression of the condition, and delaying the reoccurrence of the condition in a previously afflicted or diagnosed patient or subject.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, or amelioration of a disease, disorder, or side effect, or a decrease in the rate of advancement of a disease or disorder. The term also includes within its scope amounts effective to enhance normal physiological function.

### Compound Preparation

The compounds of Formula (I) or their salts, including pharmaceutically acceptable salts, may be made by a variety of methods, including standard chemistry. Any previously defined variable will continue to have the previously defined meaning unless otherwise indicated. Illustrative general synthetic methods are set out below and then specific compounds of Formula (I) are prepared in the Examples.

Compounds of Formula (I) may be prepared by a process involving first deprotection of a compound of Formula (II), i.e. cleavage of the ester group, followed optionally by conversion to a salt:

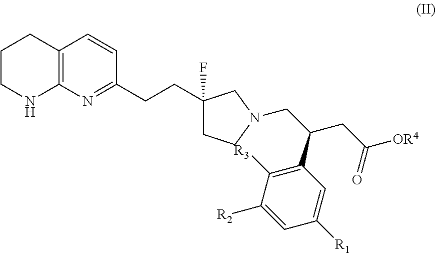

wherein R₁, R₂ and R₃ are each as hereinbefore defined, and R⁴ is a C₁-C₆ alkyl group, for example a tert-butyl, isopropyl, ethyl or methyl group. Alternatively —OR⁴ is a chiral alkoxy group for example from (−)-menthol [(1R,2S,5R)-2-isopropyl-5-methylcyclohexanol].

A further aspect of the invention provides a compound of Formula (II).

The deprotection of a compound of Formula (II) where R⁴ is methyl, menthyl or tert-butyl may be accomplished by acid hydrolysis using for example hydrochloric, hydrobromic, sulfuric, or trifluoroacetic acid, in an inert solvent, such as dichloromethane, 2-methyl-tetrahydrofuran, tetrahydrofuran, 1,4-dioxane, cyclopentyl methyl ether or water. Alternatively enzymatic hydrolysis may be used.

Alternatively the deprotection of compound of Formula (II) where R⁴ is methyl, ethyl, isopropyl or menthyl may be accomplished by base hydrolysis using for example lithium hydroxide, sodium hydroxide, potassium hydroxide in a suitable solvent, e.g. an aqueous solvent such as aqueous methanol.

After the cleavage of the ester group the resulting product may be converted to the required salt by methods well known to those skilled in the art.

In one embodiment the conversion of the zwitterion to the hydrochloride salt is achieved by treatment of a solution of the zwitterion in an inert organic solvent such as acetonitrile or acetone with an aqueous hydrochloric acid solution, concentration of the resulting salt solution and crystallisation from acetonitrile.

Compounds of Formula (II) may be obtained from compounds of Formula (III):

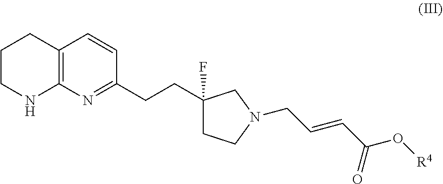

where R⁴ is as defined above, by reaction with a boronic acid compound of Formula (IV):

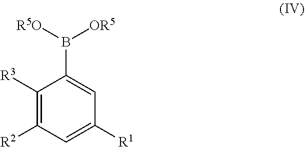

wherein R₁, R₂ and R₃ are each as hereinbefore defined, and each R₅ is hydrogen or C₁₋₄ alkyl, or both R₅ groups are linked to form a C₂₋₆ alkyl group.

Compounds of Formula (IV) may be used as the pure boronic acid (R₅═H). Alternatively a boronate ester (each R₅=alkyl group, or both R₅ are linked e.g. to form a pinacol ester), may be used, which provides the parent boronic acid in situ. The reaction between the compound of Formulae (III) and (IV) may be performed in the presence of a suitable catalyst, such as a rhodium catalyst, for example the dimer of rhodium (1,5-cyclooctadiene) chloride, [Rh(COD)Cl]₂ and an additive such as a phosphine ligand, for example bis(diphenylphosphino)-1,1′-binaphthyl (BINAP), preferably in the presence of a base, such as aqueous potassium hydroxide, at elevated temperature, such as 50-90° C., and in a water-miscible solvent, such as 1,4-dioxane. The reaction is preferably carried out under strictly anaerobic conditions, where the reaction mixture is purged with an inert gas such as nitrogen, and evacuated under reduced pressure, repeating this process of evacuation and purging with nitrogen three times. The coupling reaction in the presence of (R)-BINAP provided a diastereoisomeric mixture with a predominant isomer, for example approximately 80:20 or higher. The predominant diastereoisomer when using (R)-BINAP has the (5) configuration (as similarly shown in respect of the preparation of structurally related compounds in WO2014/154725). The diastereoisomeric ratio may be further increased to, for example greater than 99:1, by chiral HPLC, chiral SFC, or by crystallisation, at either the ester stage (compound of Formula (II)) or after conversion to the corresponding acid (compound of Formula (I)). Use of enzymatic hydrolysis for the conversion of the compound of Formula (II) to the compound of Formula (I) may also be used to increase the diastereomeric ratio and may avoid the need to use methods such as chiral HPLC.

The methyl ester group of compound (II) may be hydrolysed under the basic reaction conditions during the coupling process to provide compound (I) directly without the need of a separate hydrolysis step.

The geometry of the double bond in the compound of Formula (III) may be (E) or mixture of (E) and (Z) isomers, preferably pure (E) isomer.

Compounds of Formula (IV), where both R₅ groups and the oxygen atoms to which they are attached represent a pinacol ester, may be prepared from compounds of Formula (V):

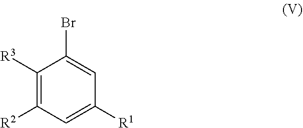

with bis(pinacolato)diboron (available from Aldrich), in the presence of a palladium catalyst, such as 1,1′-bis(diphenylphosphino)ferrocene]dichloropalladium(II) complex with dichloromethane [PdCl₂(dppf)-CH₂Cl₂ adduct] (available from Aldrich) and in the presence of potassium acetate in an inert solvent, such as 1,4-dioxane, at elevated temperature, for example 90° C., and in an inert atmosphere, such as nitrogen. Alternatively such compounds of Formula (IV) may be prepared using a palladium catalyst, such as tris(dibenzylideneacetone)dipalladium (available from Aldrich), and in the presence of a phosphine ligand, such as 2-dicyclohexylphosphino-2′,4′,6′-triisopropylbiphenyl (X-PHOS) (available from Aldrich), and in the presence of potassium acetate, in an inert solvent, such as 1,4-dioxane, at elevated temperature, for example 110° C., and in an inert atmosphere, such as nitrogen. Addition of water to the reaction mixture at the end of the reaction causes hydrolysis of the resulting pinacolato ester to provide the required boronic acid. Compounds of Formula (IV) where R₅ is hydrogen can alternatively be prepared by a three-step process involving reaction of a compound of Formula (V) with an organolithium reagent, such as n-butyllithium, in an inert solvent, such as THF or 2-methyl-tetrahydrofuran, at low temperature, such as between −60 and −78° C., and in an inert atmosphere of nitrogen or argon, followed by reaction with a trialkylborate ester such as tri(isopropyl) borate, and finally hydrolysis.

Compounds of Formula (V) may be prepared by methods described herein. For example, compounds of Formula (V) where R₁ is attached to the phenyl ring via an oxygen may be prepared from the appropriate 3-bromophenol by an alkylation reaction, for example reaction with an alkyl halide e.g. alkyl bromide or a sulfonate ester e.g. alkyl tosylate optionally in the presence of a base, in an inert solvent such as THF or DMF, and at a temperature between 20 and 60° C., or by reacting with an epoxide. Alternatively the appropriate 3-bromophenol may be alkylated via a Mitsunobu reaction using an alcohol in the presence of a phosphine e.g. triphenylphosphine and an azodicarboxylate for example diisopropyl azodicarboxylate (DIAD), in an inert solvent, such as THF and at a temperature between 0 and 25° C. For example, compounds of formula (IV) where R₁ is attached to the phenyl ring via a carbon atom may be prepared by addition of an appropriately substituted aryl lithium to a ketone to form a carbinol, which is then reduced using triethylsilane in the presence of TFA.

Compounds of Formula (III) may be obtained from compounds of Formula (VI):

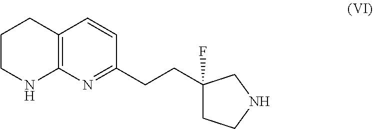 [SMILES: F[C@@]1(CCc2ccc3c(n2)NCCC3)CCNC1]

by reaction with a compound of Formula (VII)

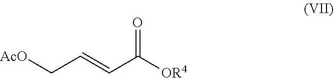

where R⁴ is as defined above, in the presence of an organic base such as N,N-diisopropylethylamine (“DIPEA”) and a suitable palladium-based catalyst, for example PdCl₂(dppf)-CH₂Cl₂ [1,1′-bis(diphenylphosphino) ferrocene]dichloropalladium(II), complex with dichloromethane, in a solvent such as dichloromethane. The compound of Formula (VII) wherein R⁴ represents tert-butyl is disclosed at page 32 of WO2014/154725. The compound of Formula (VII) wherein R⁴ represents methyl is disclosed at page 50 of WO2014/15475. The compound of Formula (VI) can be used as the parent compound, or be generated in situ from a salt, such as the dihydrochloride salt, in the presence of a tertiary amine base.

Compounds of Formula (VI) may be prepared from compounds of Formula (VIII):

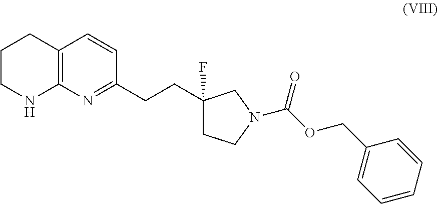 [SMILES: O=C(OCc1ccccc1)N1CC[C@@](F)(CCc2ccc3c(n2)NCCC3)C1]

by catalytic hydrogenolysis for example using a palladium catalyst deposited on carbon, in an inert solvent such as ethanol or ethyl acetate.

Compounds of Formula (VIII) may be obtained from compounds of Formula (IX):

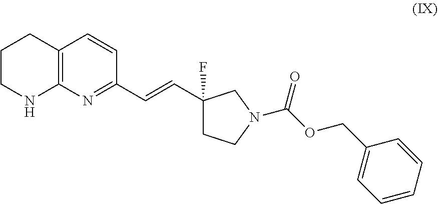 [SMILES: O=C(OCc1ccccc1)N1CC[C@@](F)(/C=C/c2ccc3c(n2)NCCC3)C1]

by diimide reduction, generated for example from benzenesulfonyl hydrazide in the presence of base such as potassium carbonate in a suitable solvent such as DMF at elevated temperature such as 130° C.

Compounds of Formula (IX) exist as geometrical isomers e.g. (E) or (Z)-form and may be used either as pure isomers or as mixtures. Compounds of Formula (IX) may be obtained from compound of Formula (X):

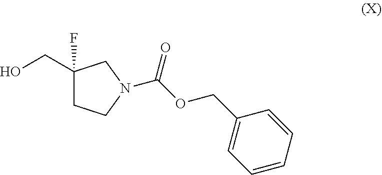 [SMILES: O=C(OCc1ccccc1)N1CC[C@@](F)(CO)C1]

which may be oxidised e.g. with sulphur trioxide in pyridine to the corresponding aldehyde of Formula (XI):

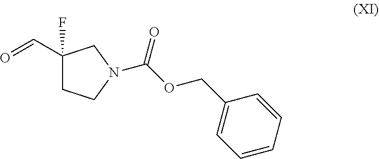 [SMILES: O=C[C@]1(F)CCN(C(=O)OCc2ccccc2)C1]

Compound of Formula (XI) may preferably be reacted in situ without prior isolation, with an ylide of Formula (XII):

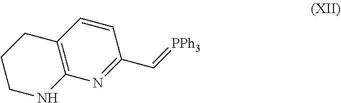

to thereby form the compound of Formula (IX), which exists as a mixture of geometrical isomers (E) and (Z). The geometrical isomers can be separated by chromatography or used in the next step as a mixture.

The overall scheme for preparation of compounds of Formula (I) is summarised below as Scheme (I):

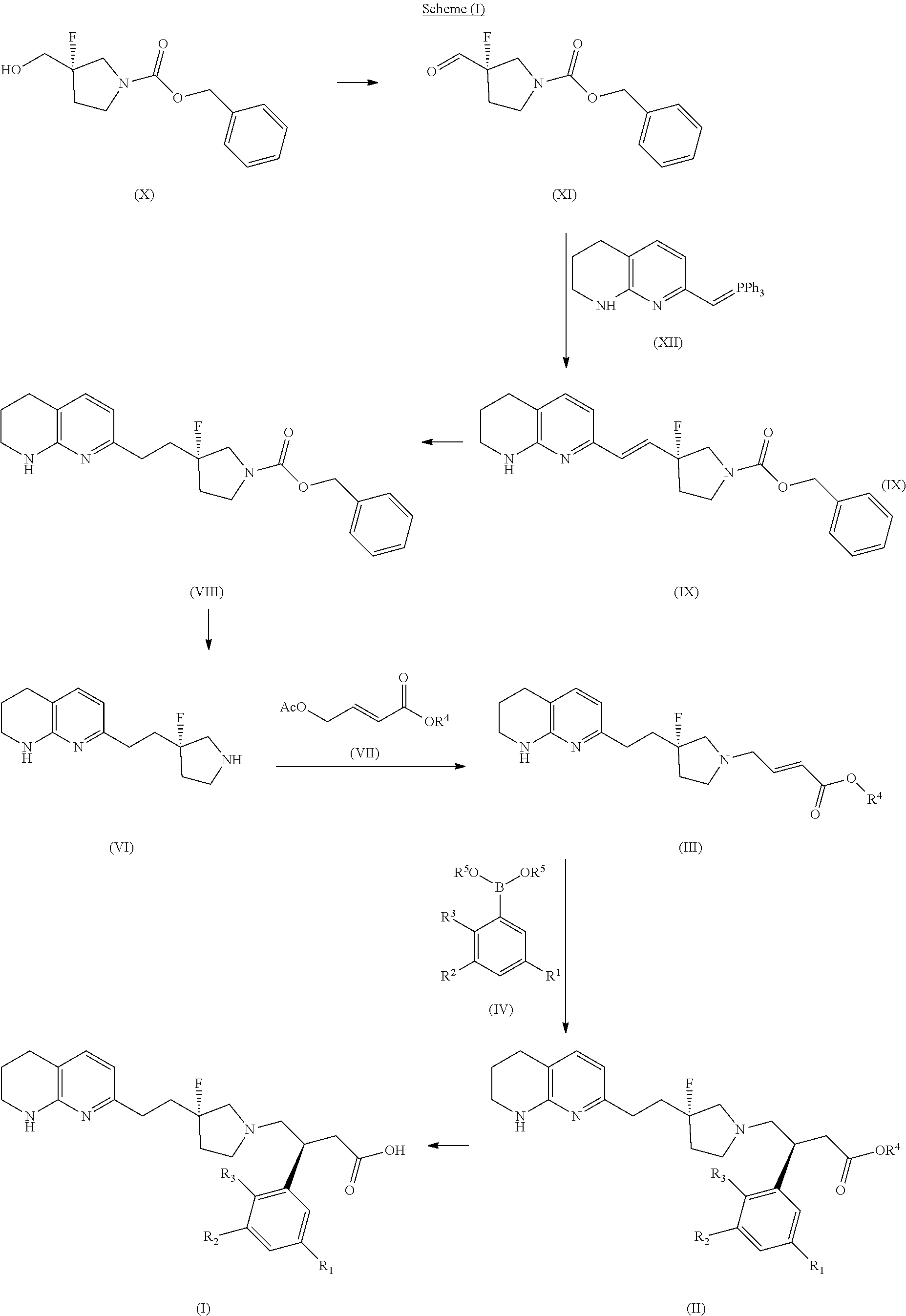

Ylides of Formula (XII) may be made starting from compounds of Formula (XIII) (available from Fluorochem):

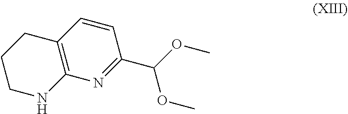

which by reaction with first hydrochloric acid followed by neutralisation with sodium bicarbonate may then be converted into an aldehyde of Formula (XIV):

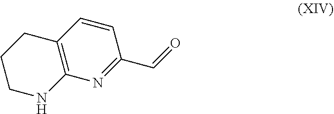 [SMILES: O=Cc1ccc2c(n1)NCCC2]

Compounds of Formula (XIV) may be reduced e.g. using sodium borohydride to the corresponding alcohol of Formula (XV):

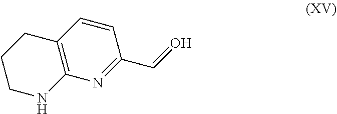 [SMILES: O=Cc1ccc2c(n1)NCCC2]

(See also the routes disclosed in US-A-20040092538 for preparation of alcohols of Formula (XV)) which may then be brominated e.g. using phosphorus tribromide to produce the corresponding bromo compound of Formula (XVI):

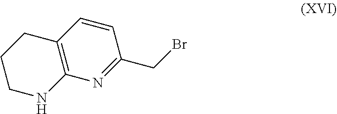 [SMILES: BrCc1ccc2c(n1)NCCC2]

Compounds of Formula (XVI) which may be converted to the triphenylphosphonium bromide (XVII) by reacting with triphenylphosphine in a solvent such as acetonitrile.

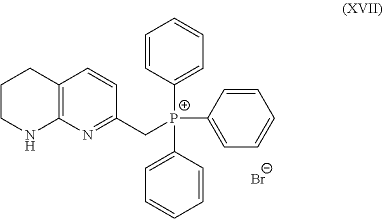 [SMILES: c1ccc([P+](Cc2ccc3c(n2)NCCC3)(c2ccccc2)c2ccccc2)cc1 ; [Br-]]

The ylide compound of Formula (XI) may be obtained by reaction of compound of Formula (XVI) with a base, such as a solution of potassium tert-butoxide in an inert solvent, such as THF. The ylide of Formula (XII) may be isolated or preferably formed in situ and reacted in the same vessel with an aldehyde of Formula (XIV) without prior isolation.

This overall scheme for preparation of ylide of Formula (XII) is summarised below as Scheme (II):

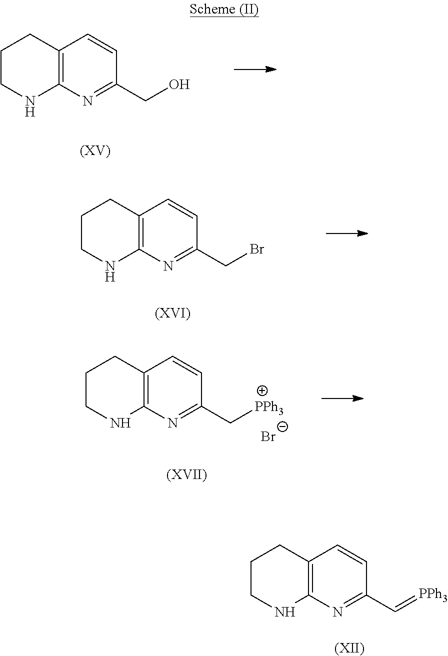

Compounds of Formula (X) may be made starting from compounds of Formula (XVIII) (available from Sigma Aldrich):

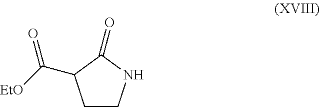 [SMILES: CCOC(=O)C1CCNC1=O]

Compounds of Formula (XVIII) may be converted by reaction with (+)-menthol [(1S,2R,5S)-2-isopropyl-5-methylcyclohexanol] (available from Alfa Aesar) with catalytic DMAP in an inert solvent, such as toluene or xylenes at elevated temperatures, preferably 100-140° C., into the corresponding (+)-menthol ester of Formula (XIX):

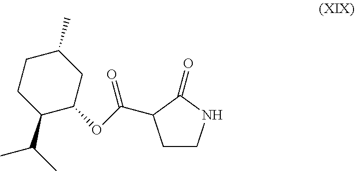 [SMILES: CC(C)[C@H]1CC[C@H](C)C[C@@H]1OC(=O)C1CCNC1=O]

Compounds of Formula (XIX) may be converted by reaction with N-fluorobenzenesulfonimide (NFSI) in the presence of a palladium catalyst, preferably 0.5 to 20 mol % of (S)-BINAP-Pd(OTf)₂(MeCN)₂ [for preparation see: Neil R. Curtis et al., Org Process Res Dev., 2015, 19 (7), pp 865-871] in the presence of a base such as 2,6-lutidine or DIPEA in a suitable solvent, such as EtOH or toluene, into the ester of Formula (XX):

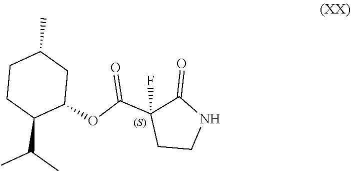

The reaction provides a diastereoisomeric mixture with a predominant isomer, for example approximately 90:10 or higher. The predominant diastereoisomer when using (S)-BINAP-Pd(OTf)₂(MeCN)₂ has the (S) configuration at the pyrrolidine stereocentre. The diastereoisomeric ratio may be further increased to, for example greater than 99:1 by crystallisation, or by chromatography.

Compounds of Formula (X) may be converted by reduction, preferably using excess borane dimethylsulfide complex in an inert solvent, such as THF, at elevated temperatures, such as 66° C., to the corresponding alcohol of Formula (XXI):

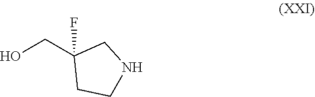

The compound of Formula (X) may be obtained by reaction of compound of Formula (XXI) with N-(benzyloxycarbonyloxy)succinimide in the presence of a base, such as excess sodium hydroxide or potassium carbonate, in a 1:1 mixture of water and water immiscible solvent, such as DCM or TBME, alternatively using benzyl chloroformate in the presence of a base, such as triethylamine or DIPEA in an inert solvent such as DCM or THF.

The overall scheme for preparation of Formula (XI) is summarised below as Scheme (III):

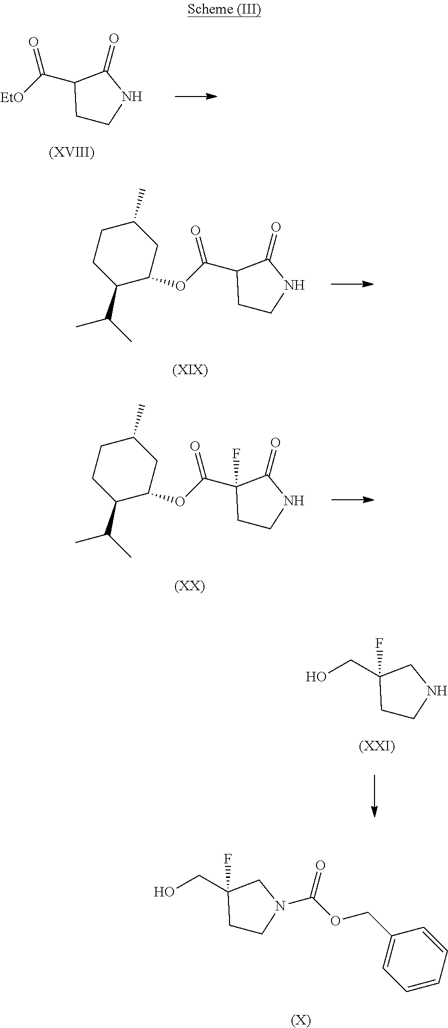

It will be appreciated that in any of the routes described above it may be advantageous to protect one or more functional groups. Examples of protecting groups and the means for their removal can be found in T. W. Greene ‘Protective Groups in Organic Synthesis’ (3rd edition, J. Wiley and Sons, 1999). Suitable amine protecting groups include acyl (e.g. acetyl, carbamate (e.g. 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid in dioxane or trifluoroacetic acid in dichloromethane) or reductively (e.g. hydrogenolysis of a benzyl or benzyloxycarbonyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis.

It will be appreciated that in any of the routes described above, the precise order of the synthetic steps by which the various groups and moieties are introduced into the molecule may be varied. It will be within the skill of the practitioner in the art to ensure that groups or moieties introduced at one stage of the process will not be affected by subsequent transformations and reactions, and to select the order of synthetic steps accordingly.

Certain compounds of Formula (IV) are also believed to be novel and therefore form a yet further aspect of the invention.

The absolute configuration of compounds of Formula (I) may be obtained following an independent enantioselective synthesis from an intermediate of known absolute configuration. Alternatively an enantiomeric pure compound of Formula (I) may be converted into a compound whose absolute configuration is known. In either case comparison of spectroscopic data, optical rotation and retention times on an analytical chiral HPLC column may be used to confirm absolute configuration. A third option where feasible is determination of absolute configuration through X-Ray crystallography.

### Methods of Use

The compounds of Formula (I) and pharmaceutically acceptable salts thereof have av integrin antagonist activity, particularly αᵥβ₆ receptor activity, and thus have potential utility in the treatment of diseases or conditions for which an αᵥβ₆ antagonist is indicated.

The present invention thus provides a compound of Formula (I) or a pharmaceutically acceptable salt thereof for use in therapy. The compound of Formula (I) or pharmaceutically acceptable salt thereof can be for use in the treatment of a disease or condition for which an αᵥβ₆ integrin antagonist is indicated.

The present invention thus provides a compound of Formula (I) or a pharmaceutically acceptable salt thereof for use in the treatment of a disease or condition for which an αᵥβ₆ integrin antagonist is indicated.

Also provided is the use of a compound of Formula (I) or a pharmaceutically acceptable salt thereof in the manufacture of a medicament for the treatment of a disease or condition for which an αᵥβ₆ integrin antagonist is indicated.

Also provided is a method of treating a disease or condition for which an αᵥβ₆ integrin antagonist is indicated in a subject in need thereof which comprises administering a therapeutically effective amount of a compound of Formula (I) or a pharmaceutically acceptable salt thereof.

Suitably the subject in need thereof is a mammal, particularly a human.

Fibrotic diseases involve the formation of excess fibrous connective tissue in an organ or tissue in a reparative or reactive process. αᵥβ₆ antagonists are believed to be useful in the treatment of a variety of such diseases or conditions including those dependent on αᵥβ₆ integrin function and on activation of transforming growth factor beta via alpha v integrins. Accordingly, in one embodiment the disease or condition for which an αᵥβ₆ antagonist is indicated is a fibrotic disease. Diseases may include but are not limited to pulmonary fibrosis (e.g. idiopathic pulmonary fibrosis, non-specific interstitial pneumonia (NSIP), usual interstitial pneumonia (UIP), Hermansky-Pudlak syndrome, progressive massive fibrosis (a complication of coal workers' pneumoconiosis), connective tissue disease-related pulmonary fibrosis, airway fibrosis in asthma and COPD, ARDS associated fibrosis, acute lung injury, radiation-induced fibrosis, familial pulmonary fibrosis, pulmonary hypertension); renal fibrosis (diabetic nephropathy, IgA nephropathy, lupus nephritis, focal segmental glomerulosclerosis (FSGS), transplant nephropathy, autoimmune nephropathy, drug-induced nephropathy, hypertension-related nephropathy, nephrogenic systemic fibrosis); liver fibrosis (virally-induced fibrosis (e.g. hepatitis C or B), autoimmune hepatitis, primary biliary cirrhosis, alcoholic liver disease, non-alcoholic fatty liver disease including non-alcoholic steatohepatitis (NASH), congential hepatic fibrosis, primary sclerosing cholangitis, drug-induced hepatitis, hepatic cirrhosis); skin fibrosis (hypertrophic scars, scleroderma, keloids, dermatomyositis, eosinophilic fasciitis, Dupytrens contracture, Ehlers-Danlos syndrome, Peyronie's disease, epidermolysis bullosa dystrophica, oral submucous fibrosis); ocular fibrosis (age-related macular degeneration (AMD), diabetic macular oedema, dry eye, glaucoma) corneal scarring, corneal injury and corneal wound healing, prevention of filter bleb scarring post trabeculectomy surgery; cardiac fibrosis (congestive heart failure, atherosclerosis, myocardial infarction, endomyocardial fibrosis, hypertrophic cardiomyopathy (HCM)) and other miscellaneous fibrotic conditions (mediastinal fibrosis, myelofibrosis, retroperitoneal fibrosis, Crohn's disease, neurofibromatosis, uterine leiomyomas (fibroids), chronic organ transplant rejection. There may be further benefit from additional inhibition of αᵥβ₁, αᵥβ₅ or αᵥβ₈ integrins

In addition, pre-cancerous lesions or cancers associated with αᵥβ₆ integrins may also be treated (these may include but are not limited to endometrial, basal cell, liver, colon, cervical, oral, pancreas, breast and ovarian cancers, Kaposi's sarcoma, Giant cell tumours and cancer associated stroma). Conditions that may derive benefit from effects on angiogenesis may also benefit (e.g. solid tumours).

The term “disease or condition for which an αᵥβ₆ antagonist is indicated”, is intended to include any or all of the above disease states.

In one embodiment the disease or condition for which an αᵥβ₆ antagonist is indicated is idiopathic pulmonary fibrosis.

In another embodiment the disease or condition for which an αᵥβ₆ antagonist is indicated is selected from corneal scarring, corneal injury and corneal wound healing.

### Compositions

While it is possible that for use in therapy, a compound of Formula (I) as well as pharmaceutically acceptable salts thereof may be administered as the raw chemical, it is common to present the active ingredient as a pharmaceutical composition.

The present invention therefore provides in a further aspect a pharmaceutical composition comprising a compound of Formula (I) or a pharmaceutically acceptable salt thereof and a pharmaceutically acceptable carrier, diluents or excipient. The compound of Formula (I) and pharmaceutically acceptable salts thereof are as described above. The carrier, diluent or excipient must be acceptable in the sense of being compatible with the other ingredients of the composition and not deleterious to the recipient thereof.

In accordance with another aspect of the invention there is also provided a process for the preparation of a pharmaceutical composition including admixing a compound of the Formula (I), or a pharmaceutically acceptable salt thereof, with a pharmaceutically acceptable carrier, diluent or excipient. The pharmaceutical composition can be for use in the treatment of any of the conditions described herein.

Further provided is a pharmaceutical composition for the treatment of diseases or conditions for which an αᵥβ₆ integrin antagonist is indicated comprising a compound of Formula (I) or a pharmaceutically acceptable salt thereof.

Further provided is a pharmaceutical composition comprising 0.05 to 1000 mg of a compound of Formula (I) or a pharmaceutical salt thereof and 0.1 to 2 g of a pharmaceutically acceptable carrier, diluent or excipient.

Since the compounds of Formula (I) are intended for use in pharmaceutical compositions it will be readily understood that they are each preferably provided in substantially pure form, for example, at least 60% pure, more suitably at least 75% pure and preferably at least 85% pure, especially at least 98% pure (% in a weight for weight basis).

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered more than once a day. Preferred unit dosage compositions are those containing a daily dose or sub-dose (for administration more than once a day), as herein above recited, or an appropriate fraction thereof, of an active ingredient.

Pharmaceutical compositions may be adapted for administration by any appropriate route, for example by the oral (including buccal or sublingual), rectal, inhaled, intranasal, topical (including buccal, sublingual or transdermal), vagina, ocular or parenteral (including subcutaneous, intramuscular, intravenous or intradermal) route. Such compositions may be prepared by any method known in the art of pharmacy, for example by bringing into association the active ingredient with the carrier or excipient.

In one embodiment the pharmaceutical composition is adapted for oral administration.

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders suitable for incorporating into tablets or capsules may be prepared by reducing the compound to a suitable fine particle size (e.g. by micronisation) and mixing with a similarly prepared pharmaceutical carrier such as an edible carbohydrate, as, for example, starch or mannitol. Flavoring, preservative, dispersing and coloring agent can also be present.

Capsules may be made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilising agent such as agaragar, calcium carbonate or sodium carbonate can also be added to improve the availability of the medicament when the capsule is ingested.

Moreover, when desired or necessary, suitable binders, glidants, lubricants, sweetening agents, flavours, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like.

Lubricants used in these dosage forms include sodium oleate, sodium stearate, magnesium stearate, sodium benzoate, sodium acetate, sodium chloride and the like.

Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavoured aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavour additive such as peppermint oil or natural sweeteners or saccharin or other artificial sweeteners, and the like can also be added.

Where appropriate, dosage unit compositions for oral administration can be microencapsulated. The formulation can also be prepared to prolong or sustain the release as for example by coating or embedding particulate material in polymers, wax or the like.

The compounds of the invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the invention may also be prepared as an amorphous molecular dispersion in a polymer matrix, such as hydroxypropylmethyl cellulose acetate succinate, using a spray-dried dispersion (SDD) process to improve the stability and solubility of the drug substance.

The compounds of the invention may also be delivered using a liquid encapsulation technology to improve properties such as bioavailability and stability, in either liquid or semi-solid filled hard capsule or soft gelatin capsule formats.

Pharmaceutical compositions adapted for transdermal administration may be presented as discrete patches intended to remain in intimate contact with the epidermis of the recipient for a prolonged period of time.

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water miscible ointment base. Alternatively, the active ingredient may be formulated in a cream with an oil-in-water cream base or a water-in-oil base. The compounds of this invention can be administered as topical eye drops. The compounds of this invention can be administered via sub-conjunctival, intracameral or intravitreal routes which would necessitate administration intervals that are longer than daily.

Pharmaceutical formulations adapted for topical administrations to the eye include eye drops wherein the active ingredient is dissolved or suspended in a suitable carrier, especially an aqueous solvent. Formulations to be administered to the eye will have ophthalmically compatible pH and osmolality. One or more ophthalmically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions.

The ocular delivery device may be designed for the controlled release of one or more therapeutic agents with multiple defined release rates and sustained dose kinetics and permeability. Controlled release may be obtained through the design of polymeric matrices incorporating different choices and properties of biodegradable/bioerodible polymers (e.g. poly(ethylene vinyl) acetate (EVA), superhydrolyzed PVA), hydroxyalkyl cellulose (HPC), methylcellulose (MC), hydroxypropyl methyl cellulose (HPMC), polycaprolactone, poly(glycolic) acid, poly(lactic) acid, polyanhydride, of polymer molecular weights, polymer crystallinity, copolymer ratios, processing conditions, surface finish, geometry, excipient addition and polymeric coatings that will enhance drug diffusion, erosion, dissolution and osmosis.

Formulations for drug delivery using ocular devices may combine one or more active agents and adjuvants appropriate for the indicated route of administration. For example, the active agents may be admixed with any pharmaceutically acceptable excipient, lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, tableted or encapsulated for conventional administration. Alternatively, the compounds may be dissolved in polyethylene glycol, propylene glycol, carboxymethyl cellulose colloidal solutions, ethanol, corn oil, peanut oil, cottonseed oil, sesame oil, tragacanth gum, and/or various buffers. The compounds may also be mixed with compositions of both biodegradable and non-biodegradable polymers and a carrier or diluent that has a time delay property. Representative examples of biodegradable compositions can include albumin, gelatin, starch, cellulose, dextrans, polysaccharides, poly (D, L-lactide), poly (D, L-lactide-co-glycolide), poly (glycolide), poly (hydroxybutyrate), poly (alkylcarbonate) and poly (orthoesters) and mixtures thereof. Representative examples of non-biodegradable polymers can include EVA copolymers, silicone rubber and poly (methylacrylate), and mixtures thereof.

Pharmaceutical compositions for ocular delivery also include in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig (2005) Adv. Drug Deliv. Rev. 3; 57:1595-639, herein incorporated by reference for purposes of its teachings of examples of polymers for use in ocular drug delivery.

Pharmaceutical compositions adapted for topical administration in the mouth include lozenges, pastilles and mouth washes.

Pharmaceutical compositions adapted for rectal administration may be presented as suppositories or as enemas.

Dosage forms for nasal or inhaled administration may conveniently be formulated as aerosols, solutions, suspensions, gels or dry powders.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the composition isotonic with the blood of the intended recipient, and aqueous and non-aqueous sterile suspensions which may include suspending agents and thickening agents. The compositions may be presented in unit dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The compounds of the invention may be administered in a long-acting parenteral (LAP) drug delivery system. Such drug delivery systems include formulations which aim to provide a slow release of drug once injected. LAP formulations may be particulate based, e.g. nano or micron sized polymeric spherical particles, which once injected would not be retrieved thus acting as a depot formulation; or small rod-like insert devices which may be retrieved if needed. Long acting particulate injectable formulations may be composed of an aqueous suspension of crystalline drug particle, where the drug has low solubility, thus providing a slow dissolution rate. Polymeric based LAP formulations are typically composed of a polymer matrix containing a drug (of hydrophilic or hydrophobic nature) homogeneously dispersed within the matrix. When LAP formulations are polymer based, the polymer widely used is poly-d,l-lactic-co-glycolic acid (PLGA) or versions thereof.

A therapeutically effective amount of a compound of Formula (I) or a pharmaceutically acceptable salt thereof (hereinafter a compound of the invention) will depend upon a number of factors including, for example, the age and weight of the subject, the precise condition requiring treatment and its severity, the nature of the formulation, and the route of administration, and will ultimately be at the discretion of the attendant physician or veterinarian.

In the pharmaceutical composition, each dosage unit for oral or parenteral administration may contain from 0.01 to 3000 mg, or 0.1 to 2000 mg, or more typically 0.5 to 1000 mg of a compound of the invention calculated as the zwitterion parent compound.

Each dosage unit for nasal or inhaled administration preferably contains from 0.001 to 50 mg, more preferably 0.01 to 5 mg, yet more preferably 1 to 50 mg, of a compound of the invention, calculated as the zwitterion parent compound.

For administration of a nebulised solution or suspension, a dosage unit typically contains from 1 to 15 mg which may suitably be delivered once daily, twice daily or more than twice daily. The compound of the invention may be provided in a dry or lyophilised powder for reconstitution in the pharmacy or by the patient, or may, for example, be provided in an aqueous saline solution.

The compounds of the invention can be administered in a daily dose (for an adult patient) of, for example, an oral or parenteral dose of 0.01 mg to 3000 mg per day, or 0.5 to 1000 mg per day or 0.5 to 300 mg per day, or 2 to 300 mg per day, or a nasal or inhaled dose of 0.001 to 50 mg per day or 0.01 to 50 mg per day, or 1 to 50 mg per day, of the compound of the invention, calculated as the zwitterion parent compound. This amount may be given in a single dose per day or more usually in a number (such as two, three, four, five or six) of sub-doses per day such that the total daily dose is the same. An effective amount of a salt thereof may be determined as a proportion of the effective amount of the compound of Formula (I) per se.

The compounds of the invention may be employed alone or in combination with other therapeutic agents. Combination therapies according to the present invention thus comprise the administration of at least one compound of Formula (I) or a pharmaceutically acceptable salt thereof, and the use of at least one other pharmaceutically active agent. Preferably, combination therapies according to the present invention comprise the administration of at least one compound of Formula (I) or a pharmaceutically acceptable salt thereof, and at least one other pharmaceutically active agent. The compound(s) of the invention and the other pharmaceutically active agent(s) may be administered together in a single pharmaceutical composition or separately and, when administered separately this may occur simultaneously or sequentially in any order. The amounts of the compound(s) of the invention and the other pharmaceutically active agent(s) and the relative timings of administration will be selected in order to achieve the desired combined therapeutic effect.

Thus in a further aspect, there is provided a combination comprising a compound of the invention and at least one other pharmaceutically active agent.

Thus in one aspect, the compound and pharmaceutical compositions according to the invention may be used in combination with or include one or more other therapeutic agents, including therapies for allergic disease, inflammatory disease, autoimmune disease, anti-fibrotic therapies and therapies for obstructive airway disease, therapies for diabetic ocular diseases, and therapies for corneal scarring, corneal injury and corneal wound healing.

Anti-allergic therapies include antigen immunotherapy (such as components and fragments of bee venom, pollen, milk, peanut, CpG motifs, collagen, other components of extracellular matrix which may be administered as oral or sublingual antigens), anti-histamines (such as cetirizine, loratidine, acrivastine, fexofenidine, chlorphenamine), and corticosteroids (such as fluticasone propionate, fluticasone furoate, beclomethasone dipropionate, budesonide, ciclesonide, mometasone furoate, triamcinolone, flunisolide, prednisolone, hydrocortisone).

Anti-inflammatory therapies include NSAIDs (such as aspirin, ibuprofen, naproxen), leukotriene modulators (such as montelukast, zafirlukast, pranlukast), and other anti-inflammatory therapies (such as iNOS inhibitors, tryptase inhibitors, IKK2 inhibitors, p38 inhibitors (losmapimod, dilmapimod), elastase inhibitors, beta2 agonists, DP1 antagonists, DP2 antagonists, p13K delta inhibitors, ITK inhibitors, LP (lysophosphatidic) inhibitors or FLAP (5-lipoxygenase activating protein) inhibitors (such as sodium 3-(3-(tert-butylthio)-1-(4-(6-ethoxypyridin-3-yl)benzyl)-5-((5-methylpyridin-2-yl)methoxy)-1H-indol-2-yl)-2,2-dimethylpropanoate); adenosine a2a agonists (such as adenosine and regadenoson), chemokine antagonists (such as CCR3 antagonists or CCR4 antagonists), mediator release inhibitors.

Therapies for autoimmune disease include DMARDS (such as methotrexate, leflunomide, azathioprine), biopharmaceutical therapies (such as anti-IgE, anti-TNF, anti-interleukins (such as anti-IL-1, anti-IL-6, anti-IL-12, anti-IL-17, anti-IL-18), receptor therapies (such as etanercept and similar agents); antigen non-specific immunotherapies (such as interferon or other cytokines/chemokines, cytokine/chemokine receptor modulators, cytokine agonists or antagonists, TLR agonists and similar agents).

Other anti-fibrotic therapies includes inhibitors of TGFβ synthesis (such as pirfenidone), tyrosine kinase inhibitors targeting the vascular endothelial growth factor (VEGF), platelet-derived growth factor (PDGF) and fibroblast growth factor (FGF) receptor kinases (such as Nintedanib (BI BF-1120) and imatinib mesylate (Gleevec)), endothelin receptor antagonists (such as ambrisentan or macitentan), antioxidants (such as N-acetylcysteine (NAC); broad-spectrum antibiotics (such as cotrimoxazole, tetracyclines (minocycline hydrochloride)), phosphodiesterase 5 (PDE5) inhibitors (such as sildenafil), anti-αvβx antibodies and drugs (such as anti-αvβ6 monoclonal antibodies such as those described in WO2003100033A2 may be used in combination, intetumumab, cilengitide) may be used in combination.

Therapies for obstructive airway diseases include bronchodilators such as short-acting β2-agonists, such as salbutamol), long-acting β2-agonists (such as salmeterol, formoterol and vilanterol), short-acting muscarinic antagonists (such as ipratropium bromide), long-acting muscarinic antagonists, (such as tiotropium, umeclidinium).

In some embodiments, treatment can also involve combination of a compound of this invention with other existing modes of treatment, for example existing agents for treatment of diabetic ocular diseases, such as anti VEGF therapeutics e.g. Lucentis®, Avastin®, and Aflibercept and steroids, e.g., triamcinolone, and steroid implants containing fluocinolone acetonide.

In some embodiments, treatment can also involve combination of a compound of this invention with other existing modes of treatment, for example existing agents for treatment of corneal scarring, corneal injury or corneal wound healing, such as Gentel®, calf blood extract, Levofloxacin®, and Ofloxacin®.

The compounds and compositions of the invention may be used to treat cancers alone or in combination with cancer therapies including chemotherapy, radiotherapy, targeted agents, immunotherapy and cell or gene therapy.

The combinations referred to above may conveniently be presented for use in the form of a pharmaceutical composition and thus pharmaceutical compositions comprising a combination as defined above together with a pharmaceutically acceptable diluent or carrier represent a further aspect of the invention. The individual compounds of such combinations may be administered either sequentially or simultaneously in separate or combined pharmaceutical compositions. Preferably, the individual compounds will be administered simultaneously in a combined pharmaceutical composition. Appropriate doses of known therapeutic agents will be readily appreciated by those skilled in the art.

It will be appreciated that when the compound of the present invention is administered in combination with one or more other therapeutically active agents normally administered by the inhaled, intravenous, oral, intranasal, ocular topical or other route that the resultant pharmaceutical composition may be administered by the same route. Alternatively, the individual components of the composition may be administered by different routes.

The present invention will now be illustrated by way of example only.

### Abbreviations

The following list provides definitions of certain abbreviations as used herein. It will be appreciated that the list is not exhaustive, but the meaning of those abbreviations not herein below defined will be readily apparent to those skilled in the art.
Ac (acetyl)
BCECF-AM (2′,7-bis-(2-carboxyethyl)-5-(and-6)-carboxyfluorescein acetoxymethyl ester)
BEH (Ethylene Bridge Hybrid Technology)
BH₃-DMS (borane dimethyl sulphide complex)
Bu (butyl)
CHAPS (3-[(3-Cholamidopropyl)dimethylammonio]-1-propanesulfonate)
Chiralcel OD-H (cellulose tris(3,5-dimethylphenylcarbamate) coated on 5 μm silica gel)
Chiralcel OJ-H (cellulose tris(4-methylbenzoate) coated on 5 μm silica gel)
Chiralpak AD-H (amylose tris(3,5-dimethylphenylcarbamate) coated on 5 μm silica gel)
Chiralpak ID (amylose tris(3-chlorophenylcarbamate) immobilised on 5 μm silica gel)
Chiralpak AS (amylose tris((S)-alpha-methylbenzylcarbamate) coated on 5 μm silica gel)
CSH (Charged Surface Hybrid Technology)
CV (column volume)
DCM (dichloromethane)
DIAD (diisopropyl azodicarboxylate)
DIPEA (diisopropylethylamine)
DMF (N,N-dimethylformamide)
DMSO (dimethylsulfoxide)
Et (ethyl)
EtOH (ethanol)
EtOAc (ethyl acetate)
FID (flame ionisation detection)
h (hour/hours)
HCl (Hydrochloric acid)
HEPES (4-(2-hydroxyethyl)-1-piperazineethanesulfonic acid)
HPLC (high performance liquid chromatography)
LCMS (liquid chromatography mass spectrometry)
LiHMDS (lithium hexamethyldisilazide)
MDAP (mass directed auto-preparative HPLC)
Me (methyl)
MeCN (acetonitrile)
MeOH (methanol)
min minute/minutes
MS (mass spectrum)
NSFI (N-Fluorobenzenesulfonimide)
PdCl₂(dppf)-CH₂Cl₂ [1,1′-Bis(diphenylphosphino)ferrocene]dichloropalladium(II), complex with dichloromethane
Ph (phenyl)
ⁱPr (isopropyl)
(R)-BINAP (R)-(+)-2,2′-Bis(diphenylphosphino)-1,1′-binaphthalene
(S)-BINAP (S)-(+)-2,2′-Bis(diphenylphosphino)-1,1′-binaphthalene
[Rh(COD)Cl]₂ [chloro(1,5-cyclooctadiene)rhodium (I) dimer]
Si (Silica)
SFC (supercritical fluid chromatography)
SPE (solid phase extraction)
TBME (tert-butyl methyl ether)
TEA (triethylamine)
TFA (trifluoroacetic acid)
THE (tetrahydrofuran)
TLC (thin layer chromatography) All references to brine refer to a saturated aqueous solution of sodium chloride.

### Experimental Details

¹H-NMR spectra were recorded at 400 MHz unless otherwise noted. Multiplicities indicated are: s=singlet, d=doublet, t=triplet, q=quartet, quint=quintet, sxt=sextet, m=multiplet, dd=doublet of doublets, dt=doublet of triplets, etc. and br indicates a broad signal.

### Analytical LCMS

Analytical LCMS was conducted on one of the following Systems A, B or C.

The UV detection to all systems was an averaged signal from wavelength of 220 nm to 350 nm and mass spectra were recorded on a mass spectrometer using alternate-scan positive and negative mode electrospray ionization.

Experimental details of LCMS Systems A, B or C as referred to herein are as follows:

System A

| Column: | 50 mm × 2.1 mm ID, 1.7 μm Acquity UPLC | | |
|---|---|---|---|
| | BEH C₁₈ column | | |
| Flow Rate: | 1 mL/min. | | |
| Temp.: | 40° C. | | |
| Solvents: | A: 10 mM ammonium bicarbonate in water adjusted to | | |
| | pH 10 with ammonia solution | | |
| | B: Acetonitrile | | |
| Gradient: | Time (min) | A % | B % |
| | 0 | 99 | 1 |
| | 1.5 | 3 | 97 |
| | 1.9 | 3 | 97 |
| | 2.0 | 0 | 100 |

System B

| Column: | 50 mm × 2.1 mm ID, 1.7 μm Acquity UPLC | | |
|---|---|---|---|
| | BEH C18 column | | |
| Flow Rate: | 1 mL/min | | |
| Temp.: | 40° C. | | |
| Solvents: | A: 0.1% v/v solution of formic acid in water | | |
| | B: 0.1% v/v solution of formic acid in acetonitrile | | |
| Gradient: | Time (min) | A % | B % |
| | 0 | 97 | 3 |
| | 1.5 | 0 | 100 |
| | 1.9 | 0 | 100 |
| | 2.0 | 97 | 3 |

System C

| Column: | 50 mm × 2.1 mm ID, 1.7 μm Acquity UPLC | | |
|---|---|---|---|
| | CSH C18 column | | |
| Flow Rate: | 1 mL/min. | | |
| Temp.: | 40° C. | | |
| Solvents: | A: 10 mM ammonium bicarbonate in water adjusted to | | |
| | pH 10 with ammonia solution | | |
| | B: Acetonitrile | | |
| Gradient: | Time (min) | A % | B % |
| | 0 | 97 | 3 |
| | 1.5 | 5 | 95 |
| | 1.9 | 5 | 95 |
| | 2.0 | 97 | 3 |

### Preparation of Intermediates

### Intermediate 1: (1S,2R,5S)-2-isopropyl-5-methylcyclohexyl 2-oxopyrrolidine-3-carboxylate (Compound XIX)

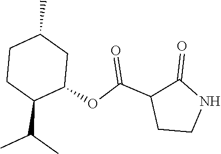 [SMILES: CC(C)[C@H]1CC[C@H](C)C[C@@H]1OC(=O)C1CCNC1=O]

A solution of (+)-Menthol (5.12 g, 32.8 mmol) (available from Alfa Aesar), ethyl 2-oxopyrrolidine-3-carboxylate (5 g, 31.8 mmol) (available from Aldrich), and DMAP (1.943 g, 15.91 mmol) in toluene (40 mL) was heated to reflux in a Dean Stark apparatus for 72 h with periodic removal of condensed toluene/ethanol mixture and replacement with equal quantity of toluene. The solution was cooled and treated with aqueous 2M hydrochloric acid solution (100 mL) and ethyl acetate (100 mL). The layers were separated and the organic layer concentrated in vacuo to give a yellow oil. The crude oil was subjected to column chromatography (silica 330 g, 0 to 100% TBME in cyclohexane over 10 CVs, visualised at 220 nm). The relevant fractions were combined and concentrated in vacuo to give the title compound (8.494 g, 100%) as a colourless oil: LCMS (System C) RT=1.17 min, ES+ ve m/z 268 (M+H)⁺.

### Intermediate 2: (S)-(1S,2R,5S)-2-isopropyl-5-methylcyclohexyl 3-fluoro-2-oxopyrrolidine-3-carboxylate (Compound XX)

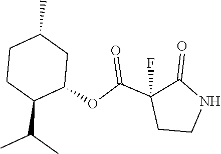 [SMILES: CC(C)[C@H]1CC[C@H](C)C[C@@H]1OC(=O)[C@]1(F)CCNC1=O]

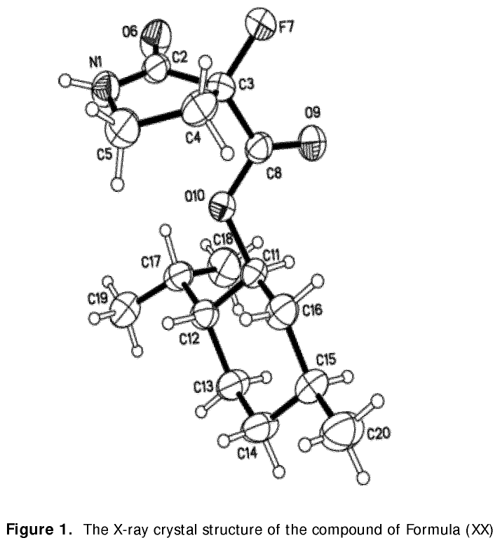

A solution of (1S,2R,5S)-2-isopropyl-5-methylcyclohexyl 2-oxopyrrolidine-3-carboxylate (compound XIX) (4.277 g, 16.00 mmol) in ethanol (100 mL) was treated with (S)-BINAP-Pd(OTf)₂(MeCN)₂ (0.089 g, 0.080 mmol) [Neil R. Curtis et al., Org Process Res Dev., 2015, 19 (7), pp 865-871] and N-fluorobenzenesulfonimide (5.55 g, 17.60 mmol) at room temperature, the reaction was cooled to 0° C. and 2,6-lutidine (0.932 mL, 8.00 mmol) was added. The reaction was allowed to warm to room temperature and stirring was continued for 4 h. The reaction was filtered through Celite with methanol washing, and the solution was concentrated in vacuo to give a yellow solid. The crude solid was dissolved in ethyl acetate (50 mL) and washed with NaOH solution (2M, 2×50 mL). The organic layer was separated, passed through a hydrophobic frit and concentrated in vacuo as a light yellow solid. The crude solid was recrystallised in TBME (100 mL) and collected by filtration to give the title compound (3.10 g, 68%) as a white crystalline solid: LCMS (System A) RT=1.22 min, ES+ ve m/z 286 (M+H)⁺; Analytical chiral HPLC on a Chiralpak IA column (250 mm×4.6 mm) RT=10.68 min, 100% eluting with 10% EtOH-heptane, flow rate 1 mL/min, detecting at 215 nm. The absolute configuration of this compound was established from an X-ray diffraction study (see FIG. 1).

### Intermediate 3: (S)-benzyl 3-fluoro-3-(hydroxymethyl)pyrrolidine-1-carboxylate (Compound X)

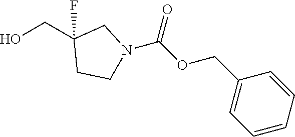 [SMILES: O=C(OCc1ccccc1)N1CC[C@@](F)(CO)C1]

(S)-(1S,2R,5S)-2-isopropyl-5-methylcyclohexyl-3-fluoro-2-oxopyrrolidine-3-carboxylate (compound XX) (500 mg, 1.752 mmol) was suspended in THE (2.5 mL) and was treated with BH₃-DMS (0.998 mL, 10.51 mmol). The resultant solution was stirred at reflux for 24 hours. The reaction mixture was cooled to 0° C. and was then slowly added to cold (0-5° C.) methanol (2.5 mL) dropwise over 15 minutes maintaining the internal temperature below 20° C.; the solution was then stirred for an hour at 10° C. Aqueous 2M HCl solution (5 mL, 10.00 mmol) was then added dropwise, maintaining the internal temperature below 20° C. Once all the HCl had been added the mixture was stirred at room temperature for 30 minutes before being heated to reflux and stirred for an hour, before being allowed to warm to room temperature. Toluene (5 mL) was added and the mixture was stirred for 10 minutes before being filtered to remove any solid. The filtrate was separated, and the lower aqueous phase was run off, and the organic phase was washed twice with 1 mL portions of aqueous 2M HCl solution. The combined aqueous phase was further washed with TBME (3×5 mL). The aqueous phases were combined, solid NaOH (406 mg, 10.16 mmol) was added portionwise, maintaining the temperature below 25° C., until the pH was 8 (pH indicator paper). The aqueous reaction mixture was diluted with TBME (7 mL) and N-(benzyloxycarbonyloxy)-succinimide (306 mg, 1.227 mmol) was added and the mixture stirred vigorously for 3 hours. The layers were separated and organic phase collected. The organic phase was washed with aqueous 2M aqueous sodium hydroxide solution (2×10 mL), aqueous 2M HCl solution (10 mL), and concentrated in vacuo giving the title compound (305 mg, 69%) as an opaque oil; LCMS (System C) RT=0.86 min, ES+ ve m/z 254 (M+H)⁺; [α]_{D}²⁰=+20 (c=1.10 in CHCl₃).

### Intermediate 4: 7-(Bromomethyl)-1,2,3,4-tetrahydro-1,8-naphthyridine (Compound XVI)

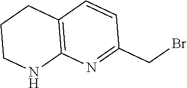 [SMILES: BrCc1ccc2c(n1)NCCC2]

Phosphorus tribromide (0.565 mL, 5.99 mmol) was added dropwise to a suspension of (5,6,7,8-tetrahydro-1,8-naphthyridin-2-yl) methanol ((Compound XV): see US20040092538) (820 mg, 4.99 mmol) in anhydrous acetonitrile (50 mL) at 0° C. under nitrogen. Upon addition a deep orange coloured precipitate formed, which turned to pale orange. The reaction mixture was stirred at 0° C. for 1 h by which time the reaction was complete. The mixture was concentrated in vacuo and the residue was partitioned between ethyl acetate (250 mL) and a saturated aqueous solution of NaHCO₃ (250 mL). The aqueous phase was further extracted with ethyl acetate (250 mL). The combined organic solutions were passed through a hydrophobic frit and then concentrated in vacuo to give the title compound (1.05 g, 93%) as a fluffy creamy solid: LCMS (System A) RT=0.95 min, ES+ ve m/z 227, 229 (M+H)⁺.

### Intermediate 5: Triphenyl((5,6,7,8-tetrahydro-1,8-naphthyridin-2-yl)methyl)phosphonium bromide (Compound (XVII))

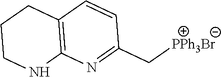

A solution of 7-(bromomethyl)-1,2,3,4-tetrahydro-1,8-naphthyridine (Compound (XVI)) (1.00 g, 4.40 mmol) in acetonitrile (98 mL) was treated with triphenylphosphine (1.270 g, 4.84 mmol) and the solution was stirred at room temperature under nitrogen overnight. The mixture was concentrated in vacuo to give a dark cream solid, which was then triturated with diethyl ether to give the title compound (2.139 g, 99%) as a pale cream solid: LCMS (System B) RT=1.23 min, ES+ ve m/z 409 (M+H)+.

### Intermediate 6: (R)-Benzyl 3-fluoro-3-(2-(5,6,7,8-tetrahydro-1,8-naphthyridin-2-yl)vinyl)pyrrolidine-1-carboxylate (Compound (IX))

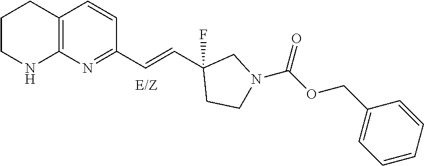 [SMILES: O=C(OCc1ccccc1)N1CC[C@@](F)(/C=C/c2ccc3c(n2)NCCC3)C1]

Prepared as disclosed in WO 2016/046225 to obtain the title compound as two geometrical isomers:

Isomer 1: a straw-coloured gum (123.4 mg, 31%), LCMS (System A) RT=1.28 min, 95%, ES+ ve m/z 382 (M+H)⁺ and

Isomer 2: a straw-coloured gum (121.5 mg, 31%), LCMS (System A) RT=1.22 min, 91%, ES+ ve m/z 382 (M+H)⁺.

### Intermediate 7: (S)-Benzyl 3-fluoro-3-(2-(5,6,7,8-tetrahydro-1,8-naphthyridin-2-yl)ethyl)pyrrolidine-1-carboxylate (Compound (VIII))

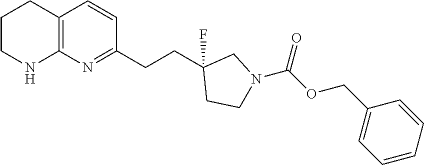 [SMILES: O=C(OCc1ccccc1)N1CC[C@@](F)(CCc2ccc3c(n2)NCCC3)C1]

Prepared as disclosed in WO 2016/046225 to give the title compound as a pale yellow gum: LCMS (System A) RT=1.24 min, 90%, ES+ ve m/z 384 (M+H)⁺.

### Intermediate 8: (S)-7-(2-(3-Fluoropyrrolidin-3-yl)ethyl)-1,2,3,4-tetrahydro-1,8-naphthyridine (Compound (VI))

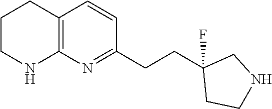 [SMILES: F[C@@]1(CCc2ccc3c(n2)NCCC3)CCNC1]

Prepared as disclosed in WO 2016/046225 to obtain the title compound as an orange oil: LCMS (System A) RT=0.79 min, 90%, ES+ ve m/z 250 (M+H)⁺.

### Intermediate 9: (S,E)-Methyl 4-(3-fluoro-3-(2-(5,6,7,8-tetrahydro-1,8-naphthyridin-2-yl)ethyl)pyrrolidin-1-yl)but-2-enoate (Compound (IIIa))

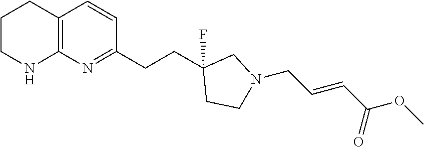 [SMILES: COC(=O)/C=C/CN1CC[C@@](F)(CCc2ccc3c(n2)NCCC3)C1]

Prepared as disclosed in WO 2016/046225 to give the title compound: LCMS (System A) RT=1.08 min, 95%, ES+ ve m/z 348 (M+H)⁺.

### Intermediate 10: (S,E)-Tert-butyl 4-(3-fluoro-3-(2-(5,6,7,8-tetrahydro-1,8-naphthyridin-2-yl)ethyl)pyrrolidin-1-yl)but-2-enoate (Compound IIIb)

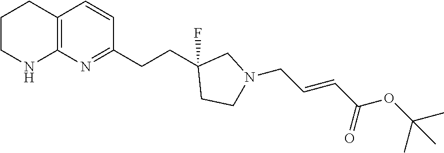 [SMILES: CC(C)(C)OC(=O)/C=C/CN1CC[C@@](F)(CCc2ccc3c(n2)NCCC3)C1]

A mixture of (E)-tert-butyl 4-acetoxybut-2-enoate (201 mg, 1.003 mmol) and (S)-7-(2-(3-fluoropyrrolidin-3-yl)ethyl)-1,2,3,4-tetrahydro-1,8-naphthyridine (Compound (VI)) (250 mg, 1.003 mmol) were stirred in DCM (2 mL) and the solution purged with nitrogen. DIPEA (0.349 mL, 2.005 mmol) and PdCl₂(dppf)-CH₂Cl₂ adduct (82 mg, 0.100 mmol) were added and the solution stirred under a nitrogen atmosphere at room temperature for 3 h. The material was loaded directly onto a column and purified by chromatography (10 g silica cartridge) eluting with 0-100% EtOAc in cyclohexane, then with 0-25% EtOH:EtOAc (3:1). The appropriate fractions were combined and evaporated to give the title compound (268 mg, 68.6%): LCMS (System B) RT=0.45 min, 87%, ES+ ve m/z 390 (M+H)⁺.

### Intermediate 11. (R)-2-((3-Bromophenoxy)methyl)tetrahydrofuran

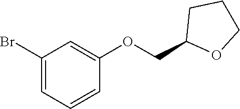 [SMILES: Brc1cccc(OC[C@H]2CCCO2)c1]

A stirred solution of 3-bromophenol (1 g, 5.78 mmol), triphenylphosphine (1.971 g, 7.51 mmol), (R)-(tetrahydrofuran-2-yl)methanol (0.708 g, 6.94 mmol) (available from Frapps) in THE (15 mL) was added DIAD (1.461 mL, 7.51 mmol) at 0° C. and stirred at 25° C. for 16 h. The reaction mixture was concentrated in vacuo, diluted with DCM (10 mL), pre-absorbed on to silica gel and purified by silica column chromatography, eluting with 5% ethyl acetate in hexane. The corresponding fractions were concentrated in vacuo to afford the title compound (1 g, 52%) as a yellow liquid: MS ES+ ve m/z 257, 259 (M+H)⁺.

### Intermediate 12. (R)-4,4,5,5-Tetramethyl-2-(3-((tetrahydrofuran-2-yl)methoxy)phenyl)-1,3,2-dioxaborolane

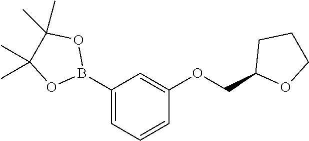 [SMILES: CC1(C)OB(c2cccc(OC[C@H]3CCCO3)c2)OC1(C)C]

A solution of (R)-2-((3-bromophenoxy)methyl)tetrahydrofuran (Intermediate 11) (1 g, 3.89 mmol), potassium acetate (1.145 g, 11.67 mmol) and bis(pinacolato)diboron (1.481 g, 5.83 mmol) in 1,4-dioxane (15 mL) was deoxygenated with argon for 15 min, then PdCl₂(dppf)-CH₂Cl₂ adduct (0.159 g, 0.194 mmol) was added. The reaction mixture was stirred at 100° C. for 18 h. The solvent was removed in vacuo to afford the crude product. The crude product was purified by silica column chromatography (10 g column), eluting with petroleum ether, and the collected fractions were concentrated in vacuo to afford the title compound (1 g, 66%) as a yellow liquid: MS ES+ ve m/z 305 (M+H)⁺.

### Intermediate 13: (S)-Methyl 4-((S)-3-fluoro-3-(2-(5,6,7,8-tetrahydro-1,8-naphthyridin-2-yl)ethyl)pyrrolidin-1-yl)-3-(3-(((R)-tetrahydrofuran-2-yl)methoxy)phenyl)butanoate

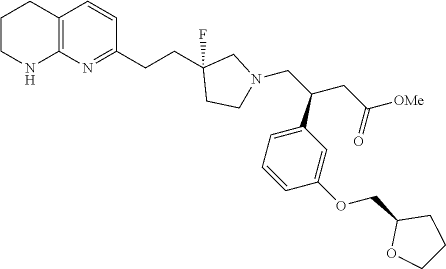 [SMILES: COC(=O)C[C@H](CN1CC[C@@](F)(CCc2ccc3c(n2)NCCC3)C1)c1cccc(OC[C@H]2CCCO2)c1]

A stirred solution of (S,E)-methyl 4-(3-fluoro-3-(2-(5,6,7,8-tetrahydro-1,8-naphthyridin-2-yl)ethyl)pyrrolidin-1-yl)but-2-enoate (compound IIIa) (250 mg, 0.720 mmol), (R)-4,4,5,5-tetramethyl-2-(3-((tetrahydrofuran-2-yl)methoxy)phenyl)-1,3,2-dioxaborolane (Intermediate 12) (657 mg, 2.159 mmol) and 3.8 M aqueous KOH solution (0.568 mL, 2.159 mmol) in 1,4-dioxane (5 mL) were deoxygenated with argon for 20 minutes. In a separate vial (R)-BINAP (53.8 mg, 0.086 mmol) and chloro(1,5-cyclooctadiene)rhodium(I) dimer (17.74 mg, 0.036 mmol) in 1,4-Dioxane (5 mL) were deoxygenated with argon for 20 minutes and added to the reaction solution, and deoxygenated with argon for further 10 minutes. The reaction mixture was stirred at 100° C. for 5 h. The reaction mixture was cooled to room temperature, solvent was removed in vacuo and subjected to silica column chromatography (40 g), eluting with linear gradient of 10-12% MeOH in DCM, and the relevant fractions were concentrated in vacuo to afford the title compound (190 mg, 50%) as a pale brown gum: MS ES+ ve m/z 526 (M+H)⁺.

### Intermediate 14. (S)-2-((3-Bromophenoxy)methyl)tetrahydrofuran

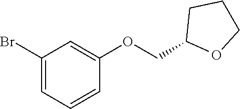 [SMILES: Brc1cccc(OC[C@@H]2CCCO2)c1]

To a stirred solution of 3-bromophenol (1 g, 5.78 mmol), triphenylphosphine (1.971 g, 7.51 mmol), and (S)-(tetrahydrofuran-2-yl)methanol (0.708 g, 6.94 mmol) (available from Alfa Aesar) in THE (15 mL) was added DIAD (1.461 mL, 7.51 mmol) at 0° C. and the solution stirred at 25° C. for 16 h. The reaction mixture was concentrated in vacuo, 1N aqueous NaOH solution (10 mL) added and extracted with DCM (2×30 mL), and purified by silica column chromatography, eluting with 5% ethyl acetate in hexane. The corresponding fractions were concentrated in vacuo to afford the title compound (1 g, 67%) as a yellow liquid: MS ES+ ve m/z 257, 259 (M+H)⁺.

### Intermediate 15. (S)-4,4,5,5-Tetramethyl-2-(3-((tetrahydrofuran-2 yl)methoxy)phenyl)-1,3,2-dioxaborolane

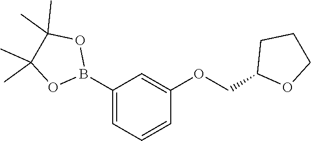 [SMILES: CC1(C)OB(c2cccc(OC[C@@H]3CCCO3)c2)OC1(C)C]

A solution of (S)-2-((3-bromophenoxy)methyl)tetrahydrofuran (Intermediate 14) (1 g, 3.89 mmol), potassium acetate (1.145 g, 11.67 mmol) and bis(pinacolato)diboron (1.481 g, 5.83 mmol) in 1,4-dioxane (15 mL) was deoxygenated with argon for 15 min, then PdCl₂(dppf)-CH₂Cl₂ adduct (0.159 g, 0.194 mmol) was added. The reaction mixture was stirred at 100° C. for 18 h. The solvent was removed in vacuo to afford the crude product. The crude product was dissolved in DCM (30 mL) then purified by silica column chromatography (50 g column), eluting with 5% EtOAc in petroleum ether, and the collected fractions were concentrated in vacuo to afford the title compound (1 g, 85%) as a yellow liquid: MS ES+ ve m/z 305 (M+H)⁺.

### Intermediate 16: (S)-Methyl 4-((S)-3-fluoro-3-(2-(5,6,7,8-tetrahydro-1,8-naphthyridin-2-yl)ethyl)pyrrolidin-1-yl)-3-(3-(((S)-tetrahydrofuran-2-yl)methoxy)phenyl)butanoate

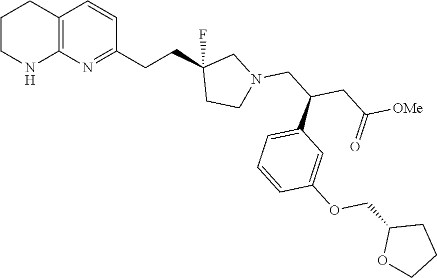 [SMILES: COC(=O)C[C@H](CN1CC[C@@](F)(CCc2ccc3c(n2)NCCC3)C1)c1cccc(OC[C@@H]2CCCO2)c1]

A stirred solution of (S,E)-methyl 4-(3-fluoro-3-(2-(5,6,7,8-tetrahydro-1,8-naphthyridin-2-yl)ethyl)pyrrolidin-1-yl)but-2-enoate (0.5 g, 1.439 mmol) (compound IIIa), (S)-4,4,5,5-tetramethyl-2-(3-((tetrahydrofuran-2-yl)methoxy)phenyl)-1,3,2-dioxaborolane (Intermediate 15) (1 g, 2.284 mmol) and 3.8 M aqueous KOH solution (1.136 mL, 4.32 mmol) in 1,4-dioxane (5 mL) was deoxygenated with argon for 20 minutes. In a separate vial (R)-BINAP (108 mg, 0.173 mmol) and chloro(1,5-cyclooctadiene)rhodium(I) dimer (35 mg, 0.072 mmol) in 1,4-Dioxane (5 mL) was deoxygenated with argon for 20 minutes and added to the reaction solution, and was deoxygenated with argon for further 10 minutes. The reaction mixture was stirred at 100° C. for 12 h. The reaction mixture was cooled to room temperature, solvent was removed in vacuo and subjected to silica column chromatography (40 g), eluting with a linear gradient of 10% MeOH in DCM, and then the relevant fractions were concentrated in vacuo to afford the title compound (300 mg, 40%) as a pale brown gum: MS ES+ ve m/z 526 (M+H)⁺.

### Intermediate 17. (R)-3-(3-Bromophenoxy)tetrahydrofuran

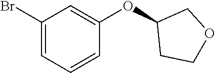 [SMILES: Brc1cccc(O[C@@H]2CCOC2)c1]

A solution of 3-bromophenol (10 g, 57.8 mmol), triphenylphosphine (22.74 g, 87 mmol), and (S)-tetrahydrofuran-3-ol (5.09 g, 57.8 mmol) in THE (100 mL) was treated at 0° C. with DIAD (11.24 mL, 57.8 mmol) and then the mixture was stirred at 25° C. for 16 h. The solvent was removed in vacuo and the residue was dissolved in DCM (100 ml), adsorbed on silica (50 g) and purified by column chromatography on silica eluting with 10% EtOAc-hexane. The fractions were concentrated in vacuo to give the title compound (8 g, 52%) as a colourless liquid: MS ES+ ve m/z 243, 245 (M+H)⁺; Analytical chiral SFC on a Chiralcel OJ-H column (250 mm×4.6 mm) RT=2.24 min, 98%, CO₂, 30% co-solvent (0.5% diethylamine in MeOH), 3 g/min, 100 Bar, 29.9° C., detecting at 272 nm.

### Intermediate 18. (R)-4,4,5,5-Tetramethyl-2-(3-((tetrahydrofuran-3-yl)oxy)phenyl)-1,3,2-dioxaborolane

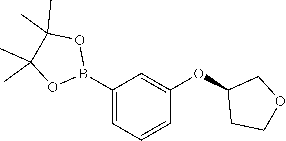 [SMILES: CC1(C)OB(c2cccc(O[C@@H]3CCOC3)c2)OC1(C)C]

A solution of (R)-3-(3-bromophenoxy)tetrahydrofuran (8 g, 33 mmol) (Intermediate 17), potassium acetate (6.46 g, 65.8 mmol) and bis(pinacolato)diboron (9.19 g, 36.2 mmol) in 1,4-dioxane (80 mL) was deoxygenated with argon gas and then treated with PdCl₂(dppf)-CH₂Cl₂ adduct (1.34 g, 1.65 mmol). The solution was deoxygenated for a further 15 min by passing argon gas through the reaction mixture and then heated to 90° C. for 16 h. The reaction mixture was cooled to room temperature, filtered through a Celite pad and washed with 1,4-dioxane (10 mL). The filtrate and washings were combined and evaporated in vacuo. The residue was adsorbed onto silica (20 g) and purified by column chromatography on silica eluting with 10% EtOAc-hexane. The fractions were concentrated in vacuo to give the title compound (6 g, 54%) as a pale yellow liquid: MS (FID) 290 (M⁺).

### Intermediate 19. (S)-Methyl 4-((S)-3-fluoro-3-(2-(5,6,7,8-tetrahydro-1,8-naphthyridin-2-yl)ethyl)pyrrolidin-1-yl)-3-(3-(((R)-tetrahydrofuran-3-yl)oxy)phenyl)butanoate

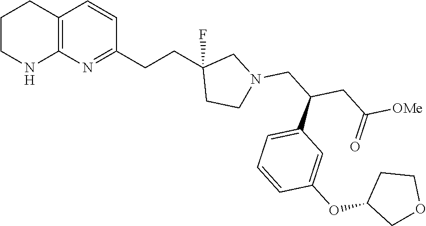 [SMILES: COC(=O)C[C@H](CN1CC[C@@](F)(CCc2ccc3c(n2)NCCC3)C1)c1cccc(O[C@@H]2CCOC2)c1]

A solution of (S,E)-methyl 4-(3-fluoro-3-(2-(5,6,7,8-tetrahydro-1,8-naphthyridin-2-yl)ethyl)pyrrolidin-1-yl)but-2-enoate (Compound IIIa) (2.5 g, 7.20 mmol), (R)-4,4,5,5-tetramethyl-2-(3-((tetrahydrofuran-3-yl)oxy)phenyl)-1,3,2-dioxaborolane (Intermediate 18) (6.26 g, 21.59 mmol), 3.8M aqueous KOH solution (4.73 mL, 17.99 mmol), (R)-BINAP (0.538 g, 0.863 mmol) and chloro(1,5-cyclooctadiene)rhodium(I) dimer (0.177 g, 0.360 mmol) in 1,4-Dioxane (12.5 mL) was stirred at 90° C. under nitrogen for 2 h. The reaction mixture was allowed to cool and separated between TBME (50 ml) and 2M aqueous HCl solution (50 ml). The aqueous phase was washed with TBME (20 ml). The aqueous phase was basified with solid sodium bicarbonate and then extracted using ethyl acetate (25 ml). The aqueous phase was extracted with more ethyl acetate (25 ml). The combined ethyl acetate extractions were washed with brine (25 ml) and dried over magnesium sulphate. The solvent was removed in vacuo to give a pale brown oil. The sample was dissolved in dichloromethane and subjected to column chromatography (100 g KPNH Silica cartridge), eluting with 0-100% EtOAc in cyclohexane. The required fractions were combined and evaporated in vacuo to give a residue which was subjected to preparative chiral HPLC on Chiralcel OD-H column (3 cm×25 cm) eluting with 30% EtOH-heptane, flow rate=30 mL/min, detecting at 215 nm, the relevant fractions were collected and concentrated in vacuo to afford the title compound (793 mg, 22%) as a gum: MS ES+ ve m/z 512 (M+H)⁺: Analytical chiral HPLC on a Chiralpak OD-H column (250 mm×4.6 mm) RT=21.27 min, 100% eluting with 50% EtOH-heptane, flow rate 1 mL/min, detecting at 215 nm.

### Intermediate 20. (S)-3-(3-Bromophenoxy)tetrahydrofuran

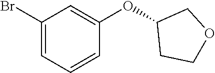 [SMILES: Brc1cccc(O[C@H]2CCOC2)c1]

To a stirred solution of 3-bromophenol (10 g, 57.8 mmol), triphenylphosphine (22.74 g, 87 mmol), (R)-tetrahydrofuran-3-ol (5.09 g, 57.8 mmol) (available from Combi Blocks) in THE (100 mL) was added DIAD (11.24 mL, 57.8 mmol) at 0° C. and the resulting mixture was stirred at 25° C. for 16 h. The solvents were removed in vacuo, the residue was diluted with DCM (100 ml) adsorbed onto silica (50 g) and purified by silica chromatography eluting with 10% EtOAc-hexane. The corresponding fractions were concentrated in vacuo and re-dissolved in DCM (100 mL), washed with 1M aqueous NaOH solution (2×25 mL) and water (50 mL), dried (Na₂SO₄) and concentrated in vacuo to afford the title compound (8 g, 56%) as a clear colourless liquid: [α]_{D}²⁵=+12 (c=1.0 in CHCl₃); Analytical chiral SFC on a YMC Amylose column (250 mm×4.6 mm) RT=2.82 min, 96%, CO₂, 25% co-solvent (0.5% diethylamine in MeOH), 3 g/min, 100 Bar, 30° C., detecting at 212 nm.

### Intermediate 21. (S)-4,4,5,5-Tetramethyl-2-(3-((tetrahydrofuran-3-yl)oxy)phenyl)-1,3,2-dioxaborolane

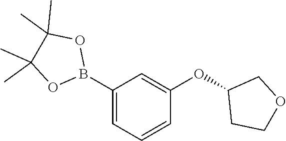 [SMILES: CC1(C)OB(c2cccc(O[C@H]3CCOC3)c2)OC1(C)C]

A solution of (S)-3-(3-bromophenoxy)tetrahydrofuran (Intermediate 20) (8 g, 33 mmol), potassium acetate (6.46 g, 65.8 mmol) and bis(pinacolato)diboron (9.19 g, 36.2 mmol) in 1,4-dioxane (80 mL) was deoxygenated with argon gas, treated with PdCl₂(dppf)-CH₂Cl₂ adduct (2.69 g, 3.29 mmol) at room temperature and the resulting mixture was deoxygenated with argon for a further 15 min. The reaction mixture was stirred at 90° C. for 16 h, cooled to room temperature and filtered through Celite. The solid was washed with 1,4-dioxane (10 mL). The filtrate washings were concentrated in vacuo, the residue was adsorbed on to silica (20 g) and purified by silica column chromatography eluting with 10% EtOAc-hexane. The corresponding fractions were collected and concentrated in vacuo to afford the title compound (6 g, 36%) as a pale brown liquid: MS ES+ ve m/z 291 (M+H)⁺.

### Intermediate 22. (S)-Methyl 4-((S)-3-fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoate

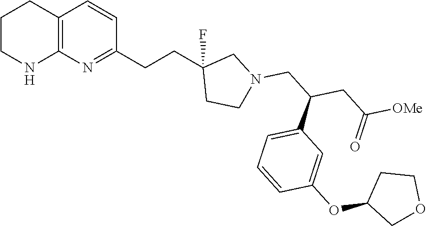 [SMILES: COC(=O)C[C@H](CN1CC[C@@](F)(CCc2ccc3c(n2)NCCC3)C1)c1cccc(O[C@H]2CCOC2)c1]

A solution of (S,E)-methyl 4-(3-fluoro-3-(2-(5,6,7,8-tetrahydro-1,8-naphthyridin-2-yl)ethyl)pyrrolidin-1-yl)but-2-enoate (Compound IIIa) (2.5 g, 7.20 mmol), (S)-4,4,5,5-tetramethyl-2-(3-((tetrahydrofuran-3-yl)oxy)phenyl)-1,3,2-dioxaborolane (Intermediate 21) (6.26 g, 21.59 mmol), 3.8M aqueous KOH solution (4.73 mL, 17.99 mmol), (R)-BINAP (0.538 g, 0.863 mmol) and chloro(1,5-cyclooctadiene)rhodium(I) dimer (0.177 g, 0.360 mmol) in 1,4-Dioxane (12.5 mL) was stirred at 90° C. under nitrogen for 2 h. The reaction mixture was allowed to cool, separated between TBME (50 ml) and 2N aqueous HCl solution (50 ml). The aqueous phase was washed with TBME (20 ml). The aqueous phase was basified with solid sodium bicarbonate and then extracted using ethyl acetate (25 ml). The aqueous phase was extracted with ethyl acetate (25 ml). The combined ethyl acetate extractions were washed with brine (25 ml) and dried over magnesium sulphate. The solvent was removed in vacuo to give a pale brown oil. The sample was dissolved in dichloromethane and subjected to column chromatography (100 g KPNH Silica cartridge), eluting with 0-100% EtOAc in cyclohexane. The required fractions were combined and evaporated in vacuo to give a residue which was subjected to preparative chiral HPLC on Chiralcel OD-H column (3 cm×25 cm) eluting with 30% EtOH-heptane, flow rate=30 mL/min, detecting at 215 nm, the relevant fraction was collected and concentrated in vacuo to afford the title compound (612 mg, 17%) as a gum: MS ES+ ve m/z 512 (M+H)⁺: Analytical chiral HPLC on a Chiralpak OD-H column (250 mm×4.6 mm) RT=21.46 min, 100% eluting with 50% EtOH-heptane, flow rate 1 mL/min, detecting at 215 nm.

### Intermediate 23. (R)-1-(3-Bromophenoxy)propan-2-ol

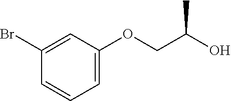

A stirred solution of 3-bromophenol (10 g, 57.8 mmol) in acetone (50 mL) was treated with (FR-2-methyloxirane (available from TCI) (16.79 g, 289 mmol) and K₂CO₃ (8.79 g, 63.6 mmol) at 0° C. in a sealed tube, and then the mixture was heated to 85° C. and stirred for 16 h. The reaction mixture was allowed to cool to room temperature and filtered. The filtrate was concentrated in vacuo, and the residue was partitioned between DCM (200 mL) and 1N aqueous NaOH solution (25 mL). The organic phase was washed with more NaOH (25 mL), water (50 mL), dried over Na₂SO₄, filtered and concentrated in vacuo to give the title compound (13 g, 94%) as a pale yellow liquid: MS ES+ ve m/z 231, 233 (M+H)⁺; Analytical chiral SFC on a YMC Amylose column (250 mm×4.6 mm) RT=2.03 min, 87%, CO₂, 20% co-solvent (0.5% diethylamine in methanol), 3 g/min, 100 Bar, 30° C., detecting at 225 nm.

### Intermediate 24: (R)-1-Bromo-3-(2-methoxypropoxy)benzene

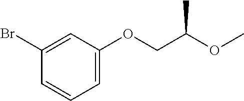

To a stirred solution of (R)-1-(3-bromophenoxy)propan-2-ol (Intermediate 23) (13 g, 56 mmol) in MeCN (130 mL) was added silver oxide (26.1 g, 113 mmol), followed by iodomethane (17.59 mL, 281 mmol) at 0° C. and the reaction mixture was stirred at 80° C. for 24 h in a sealed tube. The reaction mixture was cooled to room temperature, filtered, and the filtrate was concentrated in vacuo. The residue was diluted with DCM (10 mL), pre-adsorbed on to silica (60 g) and purified by column chromatography, eluting with 10% EtOAc in hexane. The corresponding fractions were collected and concentrated in vacuo to afford the title compound (9.50 g, 63%) as a pale yellow liquid: MS (FID) m/z 244, 246 (M)⁺.

### Intermediate 25. (R)-2-(3-(2-Methoxypropoxy)phenyl)-4,4,5,5-tetramethyl-1,3,2-dioxaborolane

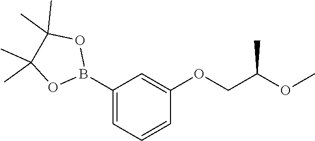

To an argon deoxygenated solution of (R)-1-bromo-3-(2-methoxypropoxy)benzene (Intermediate 24) (9.0 g, 36.7 mmol), bis(pinacolato)diboron (9.32 g, 36.7 mmol) in 1,4-dioxane (100 mL) was added potassium acetate (7.21 g, 73.4 mmol), followed by PdCl₂(dppf)-CH₂Cl₂ adduct (3.00 g, 3.67 mmol) and the resulting mixture was deoxygenated with argon for a further 20 min. The reaction mixture was heated and stirred at 90° C. for 16 h. The reaction mixture was filtered through Celite and the filtrate was concentrated in vacuo. The residue was adsorbed on Florisil and purified by silica column chromatography eluting with 2% EtOAc in hexane. The corresponding fractions were collected and concentrated in vacuo to afford the title compound (9 g, 73%) as a pale yellow liquid: MS (FID) m/z 292 (M)⁺.

### Intermediate 26. (S)-Methyl 4-((S)-3-fluoro-3-(2-(5,6,7,8-tetrahydro-1,8-naphthyridin-2-yl)ethyl)pyrrolidin-1-yl)-3-(3-((R)-2-methoxypropoxy)phenyl)butanoate

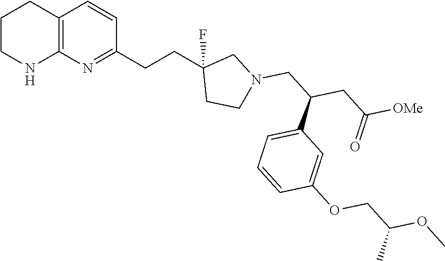 [SMILES: COC(=O)C[C@H](CN1CC[C@@](F)(CCc2ccc3c(n2)NCCC3)C1)c1cccc(OC[C@@H](C)OC)c1]

A stirred solution of (S,E)-methyl 4-(3-fluoro-3-(2-(5,6,7,8-tetrahydro-1,8-naphthyridin-2-yl)ethyl)pyrrolidin-1-yl)but-2-enoate (Compound IIIa) (400 mg, 1.151 mmol), (R)-2-(3-(2-methoxypropoxy)phenyl)-4,4,5,5-tetramethyl-1,3,2-dioxaborolane (Intermediate 25) (1009 mg, 3.45 mmol) and 3.8M aqueous potassium hydroxide solution (0.91 mL, 3.45 mmol) in 1,4-Dioxane (5 mL) was deoxygenated with argon for 15 minutes. In a separate flask a solution of (R)-BINAP (86 mg, 0.138 mmol) and Chloro(1,5-cyclooctadiene)rhodium(I) dimer (28.4 mg, 0.058 mmol) in 1,4-dioxane (3 mL) was deoxygenated with argon for 15 min. The two solutions were combined and further deoxygenated for 10 min and the reaction mixture was stirred at 90° C. for 12 h. The reaction mixture was concentrated in vacuo and subjected to silica column chromatography (40 g column) eluting with 2-4% MeOH in DCM. The corresponding fractions were collected and concentrated in vacuo to afford the title compound (350 mg, 59%): MS ES+ ve m/z 514 (M+H)⁺.

### Intermediate 27. (S)-1-(3-Bromophenoxy)propan-2-ol

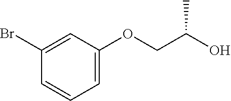

A stirred solution of 3-bromophenol (10 g, 57.8 mmol) in acetone (50 mL) was treated with (S)-2-methyloxirane (available from TCI) (20.47 mL, 289 mmol) and K₂CO₃ (8.79 g, 63.6 mmol) at 0° C. in a sealed tube, and then the mixture was heated to 85° C. and stirred for 16 h. The reaction mixture was allowed to cool to room temperature and filtered. The filtrate was concentrated in vacuo, and the residue was partitioned between DCM (10 mL) and water (10 mL). The organic phase was washed with water (10 mL), dried over Na₂SO₄, and filtered and concentrated in vacuo to give the title compound (11 g, 72%) as a yellow oil: ¹H NMR (400 MHz, CHLOROFORM-d) 7.18-7.07 (m, 3H), 6.89-6.84 (m, 1H), 4.24-4.15 (m, 1H), 3.93 (dd, J=3.2, 9.2 Hz, 1H), 3.80 (dd, J=7.6, 9.2 Hz, 1H), 1.31-1.26 (m, 3H).

### Intermediate 28: (S)-1-Bromo-3-(2-methoxypropoxy)benzene

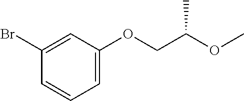

To a stirred solution of (S)-1-(3-bromophenoxy)propan-2-ol (Intermediate 27) (11 g, 47.6 mmol) in MeCN (110 mL) was added silver oxide (11.03 g, 47.6 mmol), followed by iodomethane (14.88 mL, 238 mmol) at 0° C. and the reaction mixture stirred at 80° C. for 16 h in a sealed tube. The reaction mixture was cooled to room temperature, filtered, and the filtrate was concentrated in vacuo. The residue was diluted with DCM (10 mL), pre-adsorbed on to silica (60 g) and purified by column chromatography, eluting with 10% EtOAc in hexane. The corresponding fractions were collected and concentrated in vacuo to afford the title compound (7 g, 54%) as a pale yellow liquid: MS (FID) m/z 244, 246 (M)⁺; Analytical Chiral HPLC on a Chiralpak ADH column (250 mm×4.6 mm) RT=6.07 min, 87%, eluting with 5% EtOH in hexane, flow rate 1 mL/min, detecting at 210 nm.

### Intermediate 29. (S)-2-(3-(2-Methoxypropoxy)phenyl)-4,4,5,5-tetramethyl-1,3,2-dioxaborolane

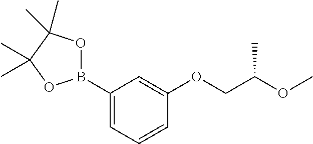

To an argon deoxygenated solution of (S)-1-bromo-3-(2-methoxypropoxy)benzene (Intermediate 28) (5.0 g, 20.4 mmol), potassium acetate (4.00 g, 40.8 mmol) and bis(pinacolato)diboron (5.70 g, 22.44 mmol) in 1,4-dioxane (100 mL) was added PdCl₂(dppf)-CH₂Cl₂ adduct (1.666 g, 2.40 mmol) and the resulting mixture was deoxygenated with argon for a further 20 min. The reaction mixture was heated and stirred at 90° C. for 16 h. The reaction mixture was cooled to room temperature filtered through Celite and the filtrate was concentrated in vacuo. The residue was washed with 1,4-dioxane, dissolved with DCM (10 mL) and purified by silica column chromatography eluting with 5% EtOAc in hexane. The corresponding fractions were collected and concentrated in vacuo to afford the title compound (3 g, 45%) as a pale yellow liquid: MS (FID) m/z 292 (M)⁺.

### Intermediate 30. (S)-methyl 4-((S)-3-fluoro-3-(2-(5,6,7,8-tetrahydro-1,8-naphthyridin-2-yl)ethyl)pyrrolidin-1-yl)-3-(3-((S)-2-methoxypropoxy)phenyl)

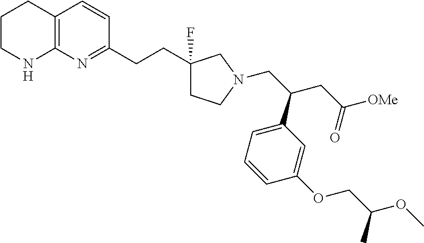 [SMILES: COC(=O)C[C@H](CN1CC[C@@](F)(CCc2ccc3c(n2)NCCC3)C1)c1cccc(OC[C@H](C)OC)c1]

A stirred solution of (S,E)-methyl 4-(3-fluoro-3-(2-(5,6,7,8-tetrahydro-1,8-naphthyridin-2-yl)ethyl)pyrrolidin-1-yl)but-2-enoate (Compound IIIa) (230 mg, 0.662 mmol), (S)-2-(3-(2-methoxypropoxy)phenyl)-4,4,5,5-tetramethyl-1,3,2-dioxaborolane (Intermediate 29) (580 mg, 1.99 mmol) and 3.8M aqueous potassium hydroxide solution (0.52 mL, 1.99 mmol) in 1,4-Dioxane (5 mL) was deoxygenated with argon for 15 minutes. In a separate flask a solution of (R)-BINAP (49.5 mg, 0.079 mmol) and Chloro(1,5-cyclooctadiene)rhodium(I) dimer (16.32 mg, 0.033 mmol) in 1,4-dioxane (5 mL) was deoxygenated with argon for 15 min. The two solutions were combined and further deoxygenated for 10 min and the reaction mixture was stirred at 90° C. for 12 h. The reaction mixture was concentrated in vacuo and subjected to silica column chromatography (40 g column) eluting with 4% MeOH in DCM. The corresponding fractions were collected and concentrated in vacuo to afford the title compound (350 mg, 59%) as a yellow gum: MS ES+ ve m/z 514 (M+H)⁺.

### Intermediate 31: Ethyl 2-(3-bromophenoxy)-2-methylpropanoate

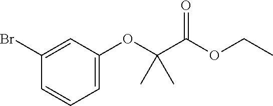 [SMILES: CCOC(=O)C(C)(C)Oc1cccc(Br)c1]

To a solution of 3-bromophenol (25 g, 145 mmol) and ethyl 2-bromo-2-methylpropanoate (23.49 mL, 159 mmol) in DMF (250 mL) was added potassium carbonate (39.9 g, 289 mmol) and the reaction mixture was stirred at 50° C. for 16 h. The reaction was cooled to 25° C. and water added (200 mL) and extracted with EtOAc (3×100 mL). The combined organic extracts were washed with water (100 mL), dried over Na₂SO₄, concentrated in vacuo and subjected to silica column chromatography eluting with 10% EtOAc in petroleum ether. The corresponding fractions were combined and concentrated in vacuo affording the title compound (16 g, 38%) as a yellow liquid: MS FID m/z 286, 288 (M)⁺.

### Intermediate 32: 2-(3-Bromophenoxy)-2-methylpropan-1-ol

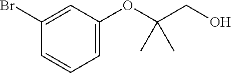

To a solution of ethyl 2-(3-bromophenoxy)-2-methylpropanoate (Intermediate 31) (16 g, 55.7 mmol) in THF (150 mL) at 0° C. was added 2M lithium borohydride in (27.9 mL, 55.7 mmol) and the resulting mixture was stirred for 8 h. The reaction mixture was cooled to 0° C. and quenched by addition of aqueous ammonium chloride solution (50 mL) and extracted with ethyl acetate (3×100 mL). The combined organic extracts were washed with water (100 mL), brine (100 mL), dried over Na₂SO₄, and concentrated in vacuo affording the title compound (10.8 g, 68%) as a pale yellow liquid: MS FID m/z 244, 246 (M)⁺.

### Intermediate 33: 1-Bromo-3-((1-methoxy-2-methylpropan-2-yl)oxy)benzene

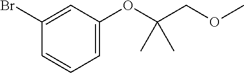

To a solution of 2-(3-bromophenoxy)-2-methylpropan-1-ol (Intermediate 32) (10 g, 40.8 mmol) in THF (100 mL) at 0° C. was added sodium hydride (60% in oil) (1.632 g, 40.8 mmol) followed by iodomethane (3.83 mL, 61.2 mmol) and it was then stirred at 25° C. for 3 h. The reaction mixture was cooled to 0° C. and quenched by addition of chilled water (50 mL), and extracted with EtOAc (3×50 mL). The combined organic extracts were dried over anhydrous Na₂SO₄ and concentrated in vacuo affording the title compound (10 g, 93%) as a yellow liquid: MS FID m/z 258, 260 (M)⁺.

### Intermediate 34: 2-(3-((1-Methoxy-2-methylpropan-2-yl)oxy)phenyl)-4,4,5,5-tetramethyl-1,3,2-dioxaborolane

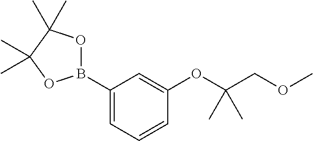

A solution of 1-bromo-3-((1-methoxy-2-methylpropan-2-yl)oxy)benzene (Intermediate 33) (10 g, 38.6 mmol), bis(pinacolato)diboron (9.80 g, 38.6 mmol) in 1,4-dioxane (100 mL) was deoxygenated with argon and potassium acetate (7.57 g, 77 mmol) was added followed by PdCl2(dppf)-CH₂Cl₂ adduct (3.15 g, 3.86 mmol) and the reaction mixture was stirred at 100° C. for 18 h. The reaction mixture was cooled to 25° C., filtered through a plug of Celite, with EtOAc (100 mL) washings, the filtrate was concentrated in vacuo and subjected to silica column chromatography, eluting with 10% EtOAc in petroleum ether. The corresponding fractions were combined and concentrated in vacuo affording the title compound (9.4 g, 75%) as a green liquid: MS FID m/z 306 (M)⁺.

### Intermediate 35: (S)-Tert-butyl 4-((S)-3-fluoro-3-(2-(5,6,7,8-tetrahydro-1,8-naphthyridin-2-yl)ethyl)pyrrolidin-1-yl)-3-(3-(oxetan-3-yloxy)phenyl)butanoate

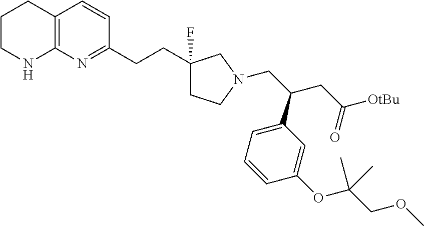 [SMILES: COCC(C)(C)Oc1cccc([C@H](CC(=O)OC(C)(C)C)CN2CC[C@@](F)(CCc3ccc4c(n3)NCCC4)C2)c1]

To a solution of (S,E)-tert-butyl 4-(3-fluoro-3-(2-(5,6,7,8-tetrahydro-1,8-naphthyridin-2-yl)ethyl)pyrrolidin-1-yl)but-2-enoate (Compound IIIb) (250 mg, 0.642 mmol) in 1,4-dioxane (5 mL) was added 2-(3-((1-methoxy-2-methylpropan-2-yl)oxy)phenyl)-4,4,5,5-tetramethyl-1,3,2-dioxaborolane (Intermediate 34) (590 mg, 1.926 mmol), and 3.8M aqueous KOH solution (0.507 mL, 1.926 mmol) and the mixture was deoxygenated with argon for 30 min. In a separate flask, a solution of (R)-BINAP (48.0 mg, 0.077 mmol) and chloro(1,5-cyclooctadiene)rhodium(I) dimer (15.82 mg, 0.032 mmol) in 1,4-dioxane (2 mL) was deoxygenated for 15 min. The two solutions were combined and heated at 100° C. for 12 h. The reaction mixture was cooled to room temperature, concentrated in vacuo and subjected to silica column chromatography (40 g column), eluting with 8% MeOH in DCM. The corresponding fractions were combined and concentrated in vacuo affording the title compound (250 mg, 68%) as a yellow gum: MS ES+ ve m/z 570 (M+H)⁺.

### Intermediate 36: 1-Bromo-3-((1,3-dimethoxypropan-2-yl)oxy)benzene

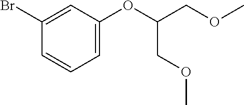

To a solution of 3-bromophenol (6 g, 34.7 mmol) and 1,3-dimethoxypropan-2-ol (5.00 g, 41.6 mmol) in THE (150 mL) was added triphenylphosphine (13.64 g, 52.0 mmol) and the reaction mixture cooled to 0° C. followed by dropwise addition of DIAD (6.74 mL, 34.7 mmol). The reaction was allowed to warm to room temperature, then stirred for 12 h. The reaction mixture was concentrated in vacuo. The obtained residue was dissolved in EtOAc (50 mL), washed with water (50 mL) and brine (50 mL), dried over Na₂SO₄, concentrated in vacuo and subjected to silica column chromatography (50 g column), eluting with 20% EtOAc in petroleum ether. The relevant fractions were combined and concentrated in vacuo affording the title compound (4.0 g, 42%) as a yellow liquid: MS ES+ ve m/z 275, 277 (M+H)⁺.

### Intermediate 37: 2-(3-Bromophenoxy)propane-1,3-diol

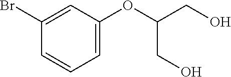 [SMILES: OCC(CO)Oc1cccc(Br)c1]

To a solution of 1-bromo-3-((1,3-dimethoxypropan-2-yl)oxy)benzene (Intermediate 36) (11 g, 40.0 mmol) in DCM (100 mL) cooled to 0° C. was added boron tribromide (11.34 mL, 120 mmol) dropwise and stirred for 0.5 h. The reaction was quenched with addition of ice water (20 mL). The layers were separated, the aqueous layer basified with 10% aqueous NaHCO₃ solution (50 mL) and extracted with DCM (3×70 mL). The combined organic layers were washed with water (50 mL) and brine (50 mL), dried over Na₂SO₄, concentrated in vacuo and subjected to silica column chromatography (25 g column) eluting with 30% EtOAc in petroleum ether. The relevant fractions were combined and concentrated in vacuo to afford the title compound (8.2 g, 83%) as an off white solid: ¹H NMR (400 MHz, CDCl₃) 7.20-7.10 (m, 3H), 6.93 (d, J=7.5 Hz, 1H), 4.43 (quin, 4.7 Hz, 1H), 3.97-3.86 (m, 4H), 3.71 (t, J=6.3 Hz, 1H), 3.51-3.43 (m, 1H).

### Intermediate 38: 2-(3-Bromophenoxy)-3-hydroxypropyl 4-methylbenzenesulfonate

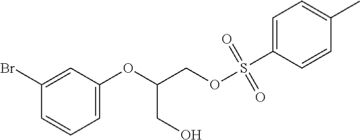 [SMILES: Cc1ccc(S(=O)(=O)OCC(CO)Oc2cccc(Br)c2)cc1]

To a solution of 2-(3-bromophenoxy)propane-1,3-diol (Intermediate 37) (8.2 g, 33.2 mmol) in THF (100 mL) cooled to 0° C. was added NaH (1.327 g, 33.2 mmol) and tosyl chloride (6.33 g, 33.2 mmol) and stirred 0.5 h. The reaction was quenched with addition of ice water (20 mL) and EtOAc (100 mL). The layers were separated and the organic layer washed with water (50 mL), brine (30 mL), dried over Na₂SO₄, concentrated in vacuo and subjected to silica column chromatography (25 g column) eluting with 30% EtOAc in petroleum ether. The relevant fractions were combined and concentrated in vacuo to afford the title compound (6.2 g, 47%) as a colourless liquid: MS ES+ ve m/z 401, 403 (M+H)⁺.

### Intermediate 39: 3-(3-Bromophenoxy)oxetane

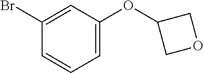 [SMILES: Brc1cccc(OC2COC2)c1]

To a solution of 2-(3-Bromophenoxy)-3-hydroxypropyl 4-methylbenzenesulfonate (Intermediate 38) (6.1 g, 15.20 mmol) in THE (60 mL) cooled to 0° C. was added NaH (0.730 g, 18.24 mmol) and stirred for 23 h at 40° C. The reaction was quenched with dropwise addition of 10% aqueous NaHCO₃ solution (15 mL) and extracted with EtOAc (3×50 mL). The combined organic layers were washed with water (20 mL), brine (20 mL), dried over Na₂SO₄, concentrated in vacuo and subjected to silica column chromatography eluting with 25% EtOAc in petroleum ether. The relevant fractions were combined and concentrated in vacuo to afford the title compound (1.3 g, 35%) as a colourless liquid: MS FID m/z 228, 230 (M)⁺.

### Intermediate 40: 4,4,5,5-Tetramethyl-2-(3-(oxetan-3-yloxy)phenyl)-1,3,2-dioxaborolane

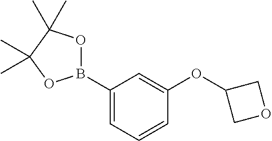 [SMILES: CC1(C)OB(c2cccc(OC3COC3)c2)OC1(C)C]

To a solution of 3-(3-bromophenoxy)oxetane (Intermediate 39) (1.0 g, 4.37 mmol) in 1,4-dioxane (20 mL) was added bis(pinacolato)diboron (1.330 g, 5.24 mmol), potassium acetate (1.285 g, 13.10 mmol). The reaction mixture was deoxygenated with N₂ for 5 min and PdCl₂(dppf)-CH₂Cl₂ adduct (0.713 g, 0.873 mmol) added. The reaction mixture was stirred at 90° C. for 12 h. The reaction mixture was concentrated in vacuo, dissolved in EtOAc (100 mL), washed with water (30 mL), brine (30 mL), dried over Na₂SO₄, concentrated in vacuo and subjected to silica column chromatography (54 g column) eluting with 20% EtOAc in petroleum ether. The relevant fractions were combined and concentrated in vacuo to afford the title compound (950 mg, 68%) as a colourless liquid: MS FID m/z 276 (M)⁺.

### Intermediate 41: (S)-Methyl 4-((S)-3-fluoro-3-(2-(5,6,7,8-tetrahydro-1,8-naphthyridin-2-yl)ethyl)pyrrolidin-1-yl)-3-(3-(oxetan-3-yloxy)phenyl)butanoate

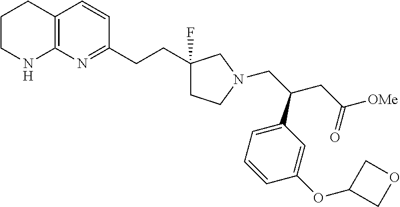 [SMILES: COC(=O)C[C@H](CN1CC[C@@](F)(CCc2ccc3c(n2)NCCC3)C1)c1cccc(OC2COC2)c1]

A solution of (S,E)-methyl 4-(3-fluoro-3-(2-(5,6,7,8-tetrahydro-1,8-naphthyridin-2-yl)ethyl)pyrrolidin-1-yl)but-2-enoate (Compound IIIa) (0.4 g, 1.155 mmol), 4,4,5,5-tetramethyl-2-(3-(oxetan-3-yloxy)phenyl)-1,3,2-dioxaborolane (Intermediate 40) (1.084 g, 3.9 mmol) and 3.8M aqueous KOH solution (0.56 mL, 3.46 mmol) in 1,4-dioxane (20 mL) was deoxygenated with argon for 20 minutes. In a separate vial, a solution of chloro(1,5-cyclooctadiene)rhodium(I)dimer (0.028 g, 0.058 mmol) and (R)-BINAP (0.144 g, 0.231 mmol) in 1,4-dioxane (10 mL) was deoxygenated with argon for 20 min. The two solutions were combined and further deoxygenated and stirred at 90° C. for 16 h. The reaction mixture was concentrated in vacuo, dissolved in 10% MeOH in DCM (10 ml) and adsorbed onto silica gel (1.2 g) and purified by silica column chromatography (40 g column), eluting with 5% MeOH in DCM. The relevant fractions were combined and concentrated in vacuo to afford the title compound (250 mg, 40%) as a brown gum: MS ES+ ve m/z 498 (M+H)⁺.

### Intermediate 42: 1-Bromo-3,5-bis(2-methoxyethoxy)benzene

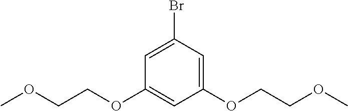

To a solution of 5-bromobenzene-1,3-diol (2.0 g, 10.58 mmol) (available from Sigma Aldrich) in DMF 10 mL) was added sequentially K₂CO₃ (5.85 g, 42.3 mmol) and 1-bromo-2-methoxyethane (3.24 g, 23.28 mmol) and the reaction mixture was stirred for 12 h. Water was added and extracted with diethylether (100 mL), dried over Na₂SO₄, concentrated in vacuo and subjected to silica column chromatography (100 g column) eluting with 10% EtOAc in hexane. The relevant fractions were combined and concentrated in vacuo affording the title compound (1.5 g, 47%) as a yellow oil: ¹H NMR (CHLOROFORM-d, 400 MHz): 6.68 (d, J=2.2 Hz, 1H), 6.45 (t, J=2.2 Hz, 1H), 4.06 (t, J=1.0 Hz, 4H), 3.71 (t, J=1.0 Hz, 4H), 3.43 (s, 6H).

### Intermediate 43: 2-(3,5-Bis(2-methoxyethoxy)phenyl)-4,4,5,5-tetramethyl-1,3,2-dioxaborolane

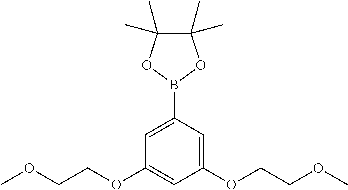

To a solution of 1-bromo-3,5-bis(2-methoxyethoxy)benzene (Intermediate 42) (3 g, 9.83 mmol) and bis(pinacolato)diboron (3.00 g, 11.80 mmol), K₂CO₃ (2.89 g, 29.5 mmol) in 1,4-dioxane (30 mL) was added PdCl₂(dppf)-CH₂Cl₂ adduct (1.606 g, 1.966 mmol) and the reaction mixture was refluxed at 100° C. overnight. The reaction mixture was concentrated in vacuo and subjected to silica column chromatography (50 g column) eluting with 30% EtOAc in hexane. The relevant fractions were combined and concentrated in vacuo affording the title compound (3.5 g, 96%) as a yellow liquid; MS ES+ ve m/z 353 (M+H)⁺.

### Intermediate 44: (S)-Methyl 3-(3,5-bis(2-methoxyethoxy)phenyl)-4-((S)-3-fluoro-3-(2-(5,6,7,8-tetrahydro-1,8-naphthyridin-2-yl)ethyl)pyrrolidin-1-yl)butanoate

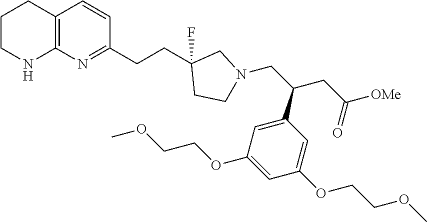 [SMILES: COCCOc1cc(OCCOC)cc([C@H](CC(=O)OC)CN2CC[C@@](F)(CCc3ccc4c(n3)NCCC4)C2)c1]

A solution of (S,E)-methyl 4-(3-fluoro-3-(2-(5,6,7,8-tetrahydro-1,8-naphthyridin-2-yl)ethyl)pyrrolidin-1-yl)but-2-enoate (Compound III a) (0.7 g, 2.015 mmol), 2-(3,5-bis(2-methoxyethoxy)phenyl)-4,4,5,5-tetramethyl-1,3,2-dioxaborolane (Intermediate 43) (2.129 g, 6.04 mmol) and 3.8M aqueous KOH (1.6 mL, 6.04 mmol) in 1,4-dioxane (5 mL) was deoxygenated with argon for 15 minutes. In a separate flask, a solution of (R)-BINAP (0.151 g, 0.242 mmol) and chloro(1,5-cyclooctadiene)rhodium(I) dimer (50 mg, 0.101 mmol) in 1,4-dioxane (2.5 mL) was deoxygenated with argon for 15 minutes. The two solutions were combined and deoxygenated for a further 10 min and stirred at 90° C. for 12 h. The reaction mixture was concentrated in vacuo and subjected to silica column chromatography (12 g column) eluting with 30% EtOAc in hexane. The relevant fractions were combined and concentrated in vacuo affording the title compound (3.5 g, 96%) as a yellow liquid; MS ES+ ve m/z 574 (M+H)⁺.

### Intermediate 45. (3-(Tetrahydrofuran-3-yl)phenyl)boronic acid

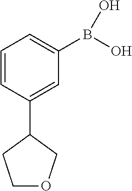 [SMILES: OB(O)c1cccc(C2CCOC2)c1]

To a stirred solution of 3-(3-iodophenyl)tetrahydrofuran (PR Guzzo et al US20120184531AA, page 52) (13 g, 47.4 mmol), triisopropylborate (17.62 mL, 76 mmol) in THE (150 mL) was added nBuLi (24.66 mL, 61.7 mmol) dropwise for 5 min at −78° C. After the addition was complete the reaction mixture was warmed to room temperature and stirred for 3 h. The reaction was quenched with 2M HCl (100 mL) and water (200 mL), EtOAc (250 mL) were added. The organic layer was separated and the aqueous layer was re-extracted with EtOAc (2×200 mL). The combined organic solutions were dried (Na₂SO₄), filtered, and concentrated under reduced pressure. The residue (10 g) was absorbed onto silica (20 g) and purified by column chromatography on silica gel (150 g), eluting with 0-50% EtOAc in petroleum ether. The fractions were combined and concentrated under reduced pressure, the residue (5 g) was washed with cold pentane (100 mL) to afford the title compound (4.2 g, 45%) as a brown gum: MS ES+ ve m/z 193 (M+H)⁺.

### Intermediate 46. (3S)-Methyl 4-((S)-3-fluoro-3-(2-(5,6,7,8-tetrahydro-1,8-naphthyridin-2-yl)ethyl)pyrrolidin-1-yl)-3-(3-(tetrahydrofuran-3-yl)phenyl)butanoate Isomer 1 and Isomer 2

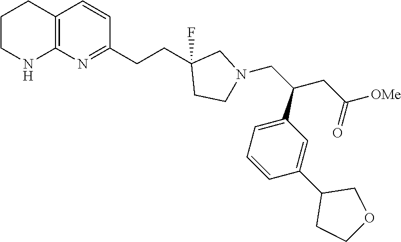 [SMILES: COC(=O)C[C@H](CN1CC[C@@](F)(CCc2ccc3c(n2)NCCC3)C1)c1cccc(C2CCOC2)c1]

A solution of (S,E)-methyl 4-(3-fluoro-3-(2-(5,6,7,8-tetrahydro-1,8-naphthyridin-2-yl)ethyl)pyrrolidin-1-yl)but-2-enoate (225 mg, 0.648 mmol) (Compound IIIa), (3-(tetrahydrofuran-3-yl)phenyl)boronic acid (Intermediate 45) (249 mg, 1.295 mmol), (R)-BINAP (48.4 mg, 0.078 mmol), chloro(1,5-cyclooctadiene)rhodium(I)dimer (15.97 mg, 0.032 mmol) and 3.8M aqueous KOH (0.341 mL, 1.295 mmol) in 1,4-dioxane (2 mL) was deoxygenated and stirred at ambient temperature for 1 h. The reaction mixture was heated to 90° C. with stirring for 1 h and left to stand overnight at ambient temperature. The reaction mixture was further heated to 90° C. for 1 h. (3-(Tetrahydrofuran-3-yl)phenyl)boronic acid (Intermediate 45) (249 mg, 1.295 mmol) was added to the reaction mixture and it was stirred for 1 h. Chloro(1,5-cyclooctadiene)rhodium(I)dimer (15.97 mg, 0.032 mmol) was added to the reaction mixture and it was stirred for 2 h. 3.8M aqueous KOH (aq) (0.341 mL, 1.295 mmol) was added to the reaction mixture and it was stirred for a further 1 h. The mixture was filtered through celite, and washed with EtOH (20 mL). The reaction mixture was concentrated in vacuo and subjected to reverse phase column chromatography (40 g C18 column) eluting with 5-70% MeCN (containing 0.1% ammonia) in 10 mM aqueous ammonium bicarbonate. The appropriate fractions were combined and concentrated in vacuo to give the crude product as a mixture of diastereomers (160 mg). This material was dissolved in EtOH (5 mL) and purified by HPLC on a Chiralcel OJ-H column (30 mm×250 mm), eluting with 80% EtOH (containing 0.2% isopropylamine) in heptane, flow-rate=20 mL/min, detecting at 215 nm. Fractions with RT=49-63 min were combined and fractions with RT=67-89 min were combined. The fractions were concentrated under reduced pressure to give the two major isomers of the title compound differing at the tetrahydrofuran asymmetric centre:

Isomer 1 (41 mg, 13%): LCMS (System A) RT=1.23 min, 88.7%, ES+ ve m/z 496 (M+H)⁺; Analytical chiral HPLC RT=25.2 min, 99.5% on a Chiralcel OJ-H column (4.6 mm×250 mm), eluting with 80% EtOH (containing 0.2% isopropylamine)-heptane, flow-rate=1 mL/min, detecting at 215 nm.

Isomer 2 (45 mg, 15%): LCMS (System A) RT=1.23 min, 90.3%, ES+ ve m/z 496 (M+H)⁺; Analytical chiral HPLC RT=32.4 min, 98.8% on a Chiralcel OJ-H column (4.6 mm×250 mm), eluting with 50% EtOH (containing 0.2% isopropylamine)-heptane, flow-rate=1 mL/min, detecting at 215 nm.

### Intermediate 47. 4-(3-Bromophenoxy)tetrahydro-2H-pyran

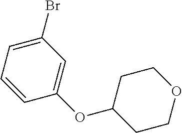 [SMILES: Brc1cccc(OC2CCOCC2)c1]

To a cooled, 5° C., solution of 3-bromophenol (7.63 g, 44.1 mmol), tetrahydro-2H-pyran-4-ol (5.41 g, 52.9 mmol) (available from Sigma Aldrich) and triphenylphosphine (23.13 g, 88 mmol) in THF (200 mL) was added DIAD (17.15 mL, 88 mmol) dropwise over 15 minutes. The reaction mixture was allowed to warm to room temperature and stirred under N₂ for 20 h. The solvent was removed in vacuo and the residue was dissolved in DCM and subjected to silica column chromatography (340 g column) eluting with 0-25% EtOAc in cyclohexane. The relevant fractions were combined and concentrated in vacuo. The residue was dissolved in TBME and washed with 2N sodium hydroxide solution. The organic phase was dried (MgSO₄) and evaporated in vacuo to give (4.89 g) as a colourless oil. The oil was dissolved in DCM and subjected to silica column chromatography (70 g column) eluting with 0-25% EtOAc in cyclohexane. The relevant fractions were combined and concentrated in vacuo to give the title compound (3.88 g, 34%) as a colourless oil; ¹H NMR (CDCl₃, 400 MHz) 7.16-7.05 (3H, m), 6.84 (1H, m), 4.50-4.42 (1H, m), 4.01-3.94 (2H, m), 3.62-3.54 (2H, m), 2.05-1.96 (2H, m), 1.83-1.73 (2H, m).

### Intermediate 48. (3-((Tetrahydro-2H-pyran-4-yl)oxy)phenyl) boronic acid

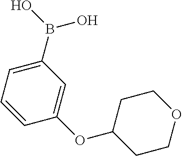 [SMILES: OB(O)c1cccc(OC2CCOCC2)c1]

A solution of 4-(3-bromophenoxy)tetrahydro-2H-pyran (3.88 g, 15.09 mmol) (Intermediate 47) in THF (70 mL) under N₂ was cooled to −70° C. To this was added dropwise 1.6M BuLi solution in hexanes (11.79 mL, 18.86 mmol) and the reaction mixture was stirred at −70° C. for 30 minutes. To this was added triisopropyl borate (5.26 mL, 22.64 mmol) and the reaction mixture was stirred at −70° C. for 1 h. The reaction mixture was allowed to warm to room temperature and then quenched with 2N aqueous hydrochloric acid (20 mL). The reaction mixture was separated between TBME (50 mL) and 2N aqueous hydrochloric acid (50 mL). The aqueous phase was extracted with TBME (50 ml). The combined organic phases were washed with brine (50 mL) and dried over magnesium sulphate. The solvent was removed in vacuo. The residue was dissolved in DCM and applied to a 100 g silica cartridge. This was eluted with a gradient of 0-100% TBME in cyclohexane over 20 minutes, followed by 0-40% methanol in TBME over 30 minutes. The relevant fractions were combined and evaporated in vacuo. The residue was treated with heptane (30 mL) and the solvent was removed in vacuo to give the title compound as a white solid (2.60 g, 78%). MS ES− ve m/z 221 (M−H).

### Intermediate 49. tert-Butyl 4-((S)-3-fluoro-3-(2-(5,6,7,8-tetrahydro-1,8-naphthyridin-2-yl)ethyl)pyrrolidin-1-yl)-3-(3-((tetrahydro-2H-pyran-4-yl)oxy)phenyl)butanoate Isomer 1 (S) and Isomer 2 (R)

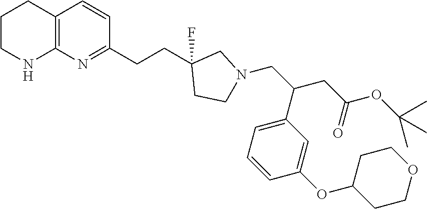 [SMILES: CC(C)(C)OC(=O)CC(CN1CC[C@@](F)(CCc2ccc3c(n2)NCCC3)C1)c1cccc(OC2CCOCC2)c1]

To a solution of (S,E)-tert-butyl 4-(3-fluoro-3-(2-(5,6,7,8-tetrahydro-1,8-naphthyridin-2-yl)ethyl)pyrrolidin-1-yl)but-2-enoate (0.62 g, 1.592 mmol) (compound IIIb), (3-((tetrahydro-2H-pyran-4-yl)oxy)phenyl)boronic acid (1.060 g, 4.78 mmol) (Intermediate 48), chloro(1,5-cyclooctadiene)rhodium(I)dimer (0.039 g, 0.080 mmol) and (R)-BINAP (0.119 g, 0.191 mmol) in 1,4-dioxane (10 mL) was added 3.8M aqueous KOH solution (1.047 mL, 3.98 mmol) and the mixture was deoxygenated. The reaction mixture was stirred at 90° C. under N₂ for 1 h. The reaction mixture was separated between ethyl acetate and 2N aqueous hydrochloric acid solution. The aqueous phase was basified with solid sodium bicarbonate. The basic phase was extracted with DCM, washed with brine and passed through a hydrophobic frit. The solvent was removed in vacuo. The residue was dissolved in DCM and subjected to silica column chromatography (20 g silica cartridge), eluting with 0-25% EtOH in EtOAc over 15 min. The relevant fractions were combined and evaporated in vacuo to give a colourless gum (684 mg). This material was dissolved in 1:1 EtOH-heptane and purified by HPLC on a Chiralcel AD-H column (30 mm×250 mm), eluting with 50% EtOH (containing 0.2% isopropylamine) in heptane, flow-rate=30 mL/min, detecting at 235 nm. Fractions with RT=7.5-12 min were combined and fractions with RT=16-21 min were combined. The relevant fractions were concentrated under reduced pressure to give the two major isomers of the title compound differing at the benzylic asymmetric centre:

Isomer 1 (S): 7.5-12 min peak (480 mg); LCMS (System C) RT=1.47 min, 100%, ES+ ve m/z 568 (M+H)⁺; Anal. Chiral HPLC RT=8.1 min, 94% on a Chiralpak AD-H column (250 mm×4.6 mm) eluting with 50% EtOH-heptane containing 0.2% isopropylamine, flow-rate=1 mL/min, detecting at 215 nm.

Isomer 2 (R): 16-21 min peak (68 mg); LCMS (System C) RT=1.46 min, 100%, ES+ ve m/z 568 (M+H)⁺; Anal. Chiral HPLC RT=17 min.

The following Intermediate compounds were prepared by similar procedures to those described above via a coupling reaction of the corresponding pinacol ester and the compound of Formula (III) wherein R⁴ represents methyl:

| Intermediate | Formula | Characterising data |
|---|---|---|
| 50 | 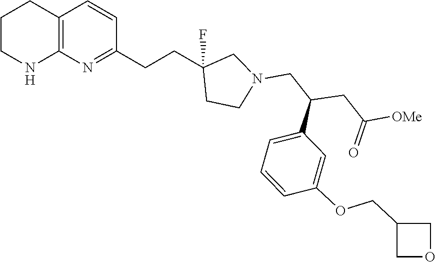 [SMILES: COC(=O)C[C@H](CN1CC[C@@](F)(CCc2ccc3c(n2)NCCC3)C1)c1cccc(OCC2COC2)c1] | MS ES+ ve m/z 512 (M + H)⁺. |
| 51 | 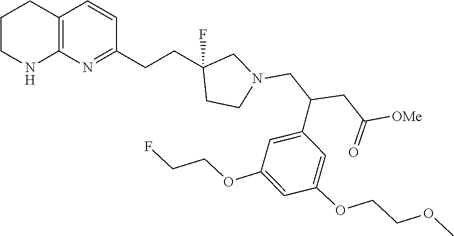 [SMILES: COCCOc1cc(OCCF)cc(C(CC(=O)OC)CN2CC[C@@](F)(CCc3ccc4c(n3)NCCC4)C2)c1] | MS ES+ ve m/z 562 (M + H)⁺. |
| 52 | 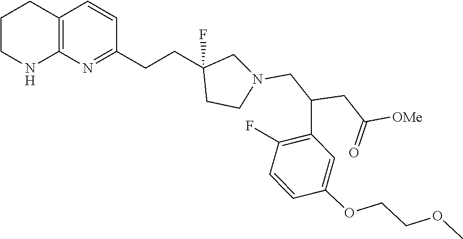 [SMILES: COCCOc1ccc(F)c(C(CC(=O)OC)CN2CC[C@@](F)(CCc3ccc4c(n3)NCCC4)C2)c1] | MS ES+ ve m/z 518 (M + H)⁺. |

### PREPARATION OF EXAMPLES

### Example 1: (S)-4-((S)-3-Fluoro-3-(2-(5,6,7,8-tetrahydro-1,8-naphthyridin-2-yl)ethyl)pyrrolidin-1-yl)-3-(3-(((R)-tetrahydrofuran-2-yl) methoxy)phenyl)butanoic acid

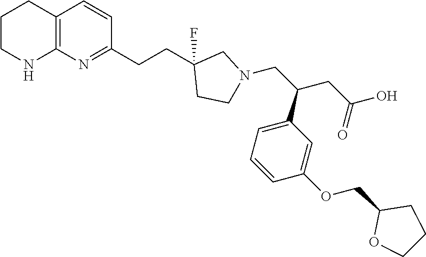 [SMILES: O=C(O)C[C@H](CN1CC[C@@](F)(CCc2ccc3c(n2)NCCC3)C1)c1cccc(OC[C@H]2CCCO2)c1]

To a solution of (S)-methyl 4-((S)-3-fluoro-3-(2-(5,6,7,8-tetrahydro-1,8-naphthyridin-2-yl)ethyl)pyrrolidin-1-yl)-3-(3-(((R)-tetrahydrofuran-2-yl)methoxy)phenyl)butanoate (Intermediate 13) (190 mg, 0.361 mmol) in THF (7.5 mL) was added a solution of LiOH (87 mg, 3.61 mmol) in water (4.8 mL) and the reaction mixture was stirred for 12 h. The reaction mixture was concentrated in vacuo and the residue (200 mg) was subjected to HPLC purification on an Xbridge C18 column (150 mm×30 mm) using a gradient of MeCN-0.1% aq TFA and a flow rate of 28 mL/min to give 150 mg and then the diastereoisomers were separated by preparative chiral SFC purification on a (R,R) Whelk-01 column (250 mm×30 mm), with 50% CO₂ and 50% MeOH (containing 0.5% diethylamine), total flow=100 g/min, back pressure=100 bar, detecting at 323 nm to give the title compound (38 mg, 25%) as an oil: LCMS (System C) RT=0.87 min, ES+ ve m/z 512 (M+H)⁺; ¹H NMR (DMSO-d₆, 600 MHz): 7.18 (t, J=8.1 Hz, 1H), 7.03 (d, J=7.3 Hz, 1H), 6.79-6.84 (m, 2H), 6.74-6.79 (m, 1H), 6.31 (br s, 1H), 6.28 (d, J=7.3 Hz, 1H), 4.11-4.20 (m, 6.2, 4.4 Hz, 1H), 3.86-3.95 (m, 2H), 3.75-3.83 (m, 1H), 3.64-3.73 (m, 1H), 3.21-3.25 (m, 2H), 3.11-3.18 (m, 1H), 2.63-2.81 (m, 6H), 2.60 (t, J=6.2 Hz, 2H), 2.46-2.56 (m, 4H), 2.41 (dd, J=15.8, 8.4 Hz, 1H), 1.79-2.03 (m, 7H), 1.75 (quin, J=5.9 Hz, 1H), 1.63-1.71 (m, 1H); Analytical chiral SFC on a (R,R) Whelk-01 column (250 mm×4.6 mm) RT=6.44 min, 99%, CO₂, 50% co-solvent (0.5% diethylamine in methanol), 4 g/min, 100 Bar, 30° C., detecting at 321 nm.

### Example 2: (S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-2-yl)methoxy)phenyl)butanoic acid

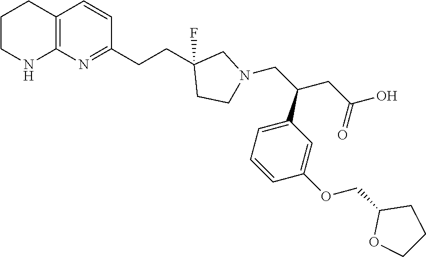 [SMILES: O=C(O)C[C@H](CN1CC[C@@](F)(CCc2ccc3c(n2)NCCC3)C1)c1cccc(OC[C@@H]2CCCO2)c1]

To a solution of (S)-methyl 4-((S)-3-fluoro-3-(2-(5,6,7,8-tetrahydro-1,8-naphthyridin-2-yl)ethyl)pyrrolidin-1-yl)-3-(3-(((S)-tetrahydrofuran-2-yl)methoxy)phenyl)butanoate (Intermediate 16) (300 mg, 0.571 mmol) in THF (7.5 mL) was added a solution of LiOH (137 mg, 5.71 mmol) in water (4.8 mL) and the reaction mixture was stirred for 12 h. The reaction mixture was concentrated in vacuo, co-distilled with MeOH to afford an off-white solid and subjected to preparative chiral SFC purification on a (R,R) Whelk-01 column (250 mm×30 mm), with 50% CO₂ and 50% MeOH (containing 0.5% diethylamine), total flow=100 g/min, back pressure=100 bar, detecting at 323 nm to give the title compound (33 mg, 11%) as an oil: LCMS (System C) RT=0.88 min, ES+ ve m/z 512 (M+H)⁺; ¹H NMR (DMSO-d₆, 600 MHz) 7.18 (t, J=8.1 Hz, 1H), 7.03 (d, J=7.3 Hz, 1H), 6.79-6.84 (m, 2H), 6.74-6.79 (m, 1H), 6.28 (d, J=7.3 Hz, 1H), 4.11-4.20 (m, 6.2, 4.4 Hz, 1H), 3.86-3.95 (m, 2H), 3.75-3.83 (m, 1H), 3.64-3.73 (m, 1H), 3.21-3.25 (m, 2H), 3.11-3.18 (m, 1H), 2.63-2.81 (m, 6H), 2.60 (t, J=6.2 Hz, 2H), 2.46-2.56 (m, 4H), 2.41 (dd, J=15.8, 8.4 Hz, 1H), 1.79-2.03 (m, 7H), 1.75 (quin, J=5.9 Hz, 1H), 1.63-1.71 (m, 1H); Analytical Chiral SFC on a (R,R) Whelk-01 column (250 mm×4.6 mm) RT=5.68 min, 98%, CO₂, 50% co-solvent (0.5% diethylamine in methanol), 4 g/min, 100 Bar, 30° C., detecting at 321 nm.

### Example 3: (S)-4-((S)-3-Fluoro-3-(2-(5,6,7,8-tetrahydro-1,8-naphthyridin-2-yl)ethyl)pyrrolidin-1-yl)-3-(3-(((R)-tetrahydrofuran-3-yl)oxy)phenyl)butanoic acid

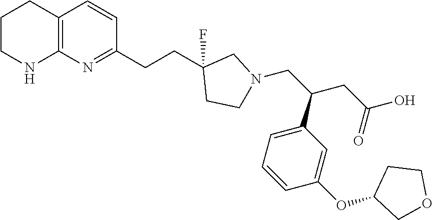 [SMILES: O=C(O)C[C@H](CN1CC[C@@](F)(CCc2ccc3c(n2)NCCC3)C1)c1cccc(O[C@@H]2CCOC2)c1]

To a solution of (S)-methyl 4-((S)-3-fluoro-3-(2-(5,6,7,8-tetrahydro-1,8-naphthyridin-2-yl)ethyl)pyrrolidin-1-yl)-3-(3-(((R)-tetrahydrofuran-3-yl)oxy)phenyl)butanoate (Intermediate 19) (793 mg, 1.550 mmol) in MeOH (3.0 mL) at 0° C. was added 2M aqueous NaOH solution (3 mL, 6.00 mmol) and the reaction mixture was stirred at room temperature for 4 hours. The reaction mixture was separated between water (5 ml) and TBME (7 ml). The aqueous phase was washed with TBME (5 ml). The aqueous phase was neutralised to pH 7.5 using 2M aqueous HCl solution and extracted with DCM (2×5 ml). The combined DCM phases were washed with brine (5 ml) and dried over magnesium sulphate and concentrated in vacuo to give the title compound (494 mg, 64% yield) as a white foam: LCMS (System C) RT=0.82 min, ES+ ve m/z 498 (M+H)⁺; ¹H NMR (400 MHz, DMSO-d₆) 12.2 (br., 1H), 7.20 (t, J=7.8 Hz, 1H), 7.08 (d, J=7.3 Hz, 1H), 6.88-6.79 (m, 2H), 6.75 (dd, J=2.3, 8.1 Hz, 1H), 6.41 (br. s., 1H), 6.32 (d, J=7.1 Hz, 1H), 5.07-4.94 (m, 1H), 3.95-3.70 (m, 4H), 3.28-3.12 (m, obscured by water), 2.93-2.65 (m, 4H), 2.64-2.47 (m, obscured by DMSO), 2.43 (dd, J=8.5, 15.8 Hz, 1H), 2.27-2.16 (m, 1H), 2.05-1.83 (m, 5H), 1.81-1.69 (m, 2H); [α]_{D}²³=+84 (c=0.5 in EtOH).

### Example 4: (S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoic acid

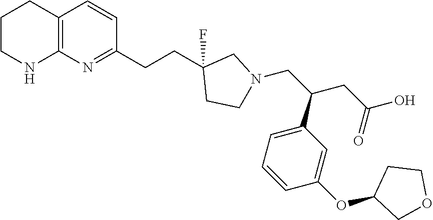 [SMILES: O=C(O)C[C@H](CN1CC[C@@](F)(CCc2ccc3c(n2)NCCC3)C1)c1cccc(O[C@H]2CCOC2)c1]

To a solution of (S)-methyl 4-((S)-3-fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoate (Intermediate 22) (612 mg, 1.196 mmol) in Methanol (2.0 mL) at 0° C. was added 2M aqueous NaOH solution (2.057 ml, 4.11 mmol) and the reaction mixture was stirred at room temperature for 4 hours. The reaction mixture was separated between water (5 ml) and TBME (7 ml). The aqueous phase was washed with TBME (5 ml). The aqueous phase was neutralised (pH 7.5) using 2M aqueous HCl solution and extracted with DCM (2×5 ml). The combined DCM phases were washed with brine (5 ml) and dried over magnesium sulphate. The solvent was removed in vacuo to give the product as a white foam (316 mg). The combined TBME phases were washed with 2M aqueous NaOH solution (50 ml) and the basic phase was added to the aqueous phase from above. The pH was adjusted to 7.5 using 2M aqueous HCl solution and extracted with DCM (2×50 ml). The combined DCM phases were dried over magnesium sulphate and concentrated in vacuo to give the product as a white foam (195 mg). The two product batches were combined to give the title compound (511 mg, 86% yield) as a white foam.

LCMS (System C) RT=0.82 min, ES+ ve m/z 498 (M+H)⁺; ¹H NMR (400 MHz, DMSO-d₆) 7.20 (t, J=8.0 Hz, 1H), 7.07 (d, J=7.1 Hz, 1H), 6.86-6.79 (m, 2H), 6.75 (dd, J=2.0, 8.1 Hz, 1H), 6.38 (br. s., 1H), 6.31 (d, J=7.3 Hz, 1H), 5.01 (m, 1H), 3.93-3.71 (m, 4H), 3.27-3.12 (m, obscured by water), 2.90-2.66 (m, 4H), 2.64-2.47 (m, obscured by DMSO), 2.43 (dd, J=8.5, 15.8 Hz, 2H), 2.28-2.16 (m, 1H), 2.06-1.83 (m, 5H), 1.80-1.70 (m, 2H); [α]_{D}²³=+95 (c=1.0 in EtOH).

### Example 5: ((S)-4-((S)-3-Fluoro-3-(2-(5,6,7,8-tetrahydro-1,8-naphthyridin-2-yl)ethyl)pyrrolidin-1-yl)-3-(3-((R)-2-methoxypropoxy)phenyl)butanoic acid

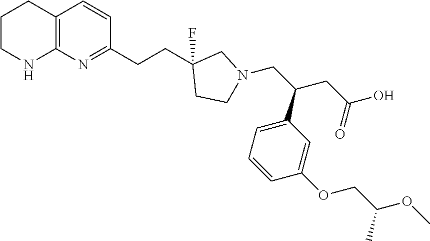 [SMILES: CO[C@H](C)COc1cccc([C@H](CC(=O)O)CN2CC[C@@](F)(CCc3ccc4c(n3)NCCC4)C2)c1]

To a solution of (R)-methyl 4-((S)-3-fluoro-3-(2-(5,6,7,8-tetrahydro-1,8-naphthyridin-2-yl)ethyl)pyrrolidin-1-yl)-3-(3-((R)-2-methoxypropoxy)phenyl)butanoate (Intermediate 26) (350 mg, 0.681 mmol) in THE (3 mL) was added a solution of LiOH (65.3 mg, 2.73 mmol) in water (2 mL) and the reaction mixture was stirred for 12 h. The reaction mixture was concentrated in vacuo, co-distilled with MeOH and subjected to preparative HPLC purification on a Kinetex acetonitrile column (150 mm×19 mm), eluting with 10 to 100% MeCN in 10 mM Ammonium bicarbonate solution over 13 min, flow rate=18 mL/min, and the relevant fractions were concentrated in vacuo to give the title compound (38 mg, 11%) as a yellow solid: LCMS (System C) RT=0.86 min, ES+ ve m/z 500 (M+H)⁺; ¹H NMR (DMSO-d₆, 400 MHz) 7.18 (t, J=8.0 Hz, 1H), 7.03 (d, J=7.3 Hz, 1H), 6.73-6.85 (m, 3H), 6.26-6.32 (m, 2H), 3.86-3.96 (m, 2H), 3.60-3.70 (m, 1H), 3.20-3.30 (m, obscured by water), 3.09-3.19 (m, 1H), 2.65-2.86 (m, 5H), 2.61 (t, J=6.1 Hz, 2H), 2.37-2.47 (m, 1H), 1.80-2.06 (m, 4H), 1.70-1.80 (m, 2H), 1.18 (d, J=6.3 Hz, 3H); [α]_{D}²³=+75 (c=1.0, EtOH).

### Example 6: ((S)-4-((S)-3-Fluoro-3-(2-(5,6,7,8-tetrahydro-1,8-naphthyridin-2-yl)ethyl)pyrrolidin-1-yl)-3-(3-((S)-2-methoxypropoxy)phenyl)butanoic acid

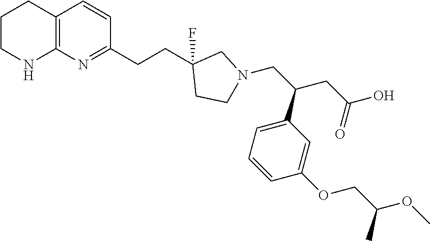 [SMILES: CO[C@@H](C)COc1cccc([C@H](CC(=O)O)CN2CC[C@@](F)(CCc3ccc4c(n3)NCCC4)C2)c1]

To a solution of (S)-methyl 4-((S)-3-fluoro-3-(2-(5,6,7,8-tetrahydro-1,8-naphthyridin-2-yl)ethyl)pyrrolidin-1-yl)-3-(3-((S)-2-methoxypropoxy)phenyl)butanoate (Intermediate 30) (230 mg, 0.448 mmol) in THE (3 mL) was added a solution of LiOH (42.9 mg, 1.79 mmol) in water (2 mL) and the reaction mixture was stirred for 12 h. The reaction mixture was concentrated in vacuo, co-distilled with MeOH and subjected to preparative HPLC purification on an Xbridge C18 column (150 mm×19 mm), eluting with 0 to 55% MeCN/MeOH (1:1) in 5 mM Ammonium bicarbonate solution, and the relevant fractions were concentrated in vacuo to give the title compound (63 mg, 28%) as a yellow solid: LCMS (System C) RT=0.84 min, ES+ ve m/z 500 (M+; ¹H NMR (DMSO-d₆, 400 MHz) 7.18 (t, J=8.0 Hz, 1H), 7.03 (d, J=7.3 Hz, 1H), 6.73-6.85 (m, 3H), 6.28 (d, J=7.3 Hz, 2H), 3.84-3.96 (m, 2H), 3.60-3.71 (m, 1H), 3.20-3.30 (m, 5H), 3.09-3.20 (m, 1H), 2.55-2.85 (m, 8H), 2.31-2.47 (m, 2H), 1.81-2.07 (m, 4H), 1.70-1.79 (m, 3H), 1.17 (d, J=6.3 Hz, 3H); [α]_{D}²³=+68 (c=1.0, EtOH).

### Example 7: (S)-4-((S)-3-Fluoro-3-(2-(5,6,7,8-tetrahydro-1,8-naphthyridin-2-yl)ethyl)pyrrolidin-1-yl)-3-(3-((1-methoxy-2-methylpropan-2-yl)oxy)phenyl)butanoic acid

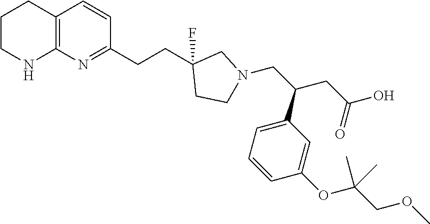 [SMILES: COCC(C)(C)Oc1cccc([C@H](CC(=O)O)CN2CC[C@@](F)(CCc3ccc4c(n3)NCCC4)C2)c1]

To a solution of (S)-tert-butyl 4-((S)-3-fluoro-3-(2-(5,6,7,8-tetrahydro-1,8-naphthyridin-2-yl)ethyl)pyrrolidin-1-yl)-3-(3-((1-methoxy-2-methylpropan-2-yl)oxy)phenyl)butanoate (Intermediate 35) (250 mg, 0.439 mmol) in DCM (5 mL) was added TFA (0.17 mL, 2.195 mmol) at 0° C. and the reaction mixture was stirred for 5 h. The reaction mixture was concentrated in vacuo and subjected to preparative HPLC purification on a Kromasil phenyl column (150 mm×25 mm), eluting with 0 to 50% MeCN in 10 mM Ammonium bicarbonate solution, flow rate=20 mL/min, and the relevant fractions were concentrated in vacuo to give the title compound (41 mg, 17%) as a brown solid: LCMS (System C) RT=0.88 min, ES+ ve m/z 514 (M+H)⁺; ¹H NMR (DMSO-d₆, 400 MHz) 7.19 (t, J=7.8 Hz, 1H), 7.06 (d, J=7.3 Hz, 1H), 6.97 (d, J=7.6 Hz, 1H), 6.85 (s, 1H), 6.81 (d, J=7.8 Hz, 1H), 6.36 (br. s., 1H), 6.30 (d, J=7.3 Hz, 1H), 3.32 (s, 3H), 3.21-3.27 (m, 2H), 3.11-3.20 (m, J=6.3 Hz, 1H), 2.66-2.88 (m, 5H), 2.61 (t, J=5.9 Hz, 2H), 2.53-2.57 (m, 5H), 2.41 (dd, J=15.7, 8.8 Hz, 1H), 1.81-2.04 (m, 5H), 1.70-1.79 (m, 2H), 1.21 (s, 6H); [α]_{D}²³=+42 (c=0.5, EtOH).

### Example 8: (S)-4-((S)-3-Fluoro-3-(2-(5,6,7,8-tetrahydro-1,8-naphthyridin-2-yl)ethyl)pyrrolidin-1-yl)-3-(3-(oxetan-3-yloxy)phenyl)butanoic acid

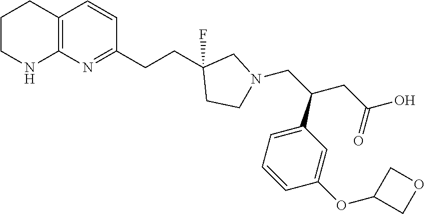 [SMILES: O=C(O)C[C@H](CN1CC[C@@](F)(CCc2ccc3c(n2)NCCC3)C1)c1cccc(OC2COC2)c1]

To a solution of (S)-methyl 4-((S)-3-fluoro-3-(2-(5,6,7,8-tetrahydro-1,8-naphthyridin-2-yl)ethyl)pyrrolidin-1-yl)-3-(3-(oxetan-3-yloxy)phenyl)butanoate (Intermediate 41) (150 mg, 0.301 mmol) in THE (8 mL) was added a solution of LiOH (36.1 mg, 1.507 mmol) in water (1.6 mL) and the reaction mixture was stirred for 18 h to give batch 1. To a solution of (S)-methyl 4-((S)-3-fluoro-3-(2-(5,6,7,8-tetrahydro-1,8-naphthyridin-2-yl)ethyl)pyrrolidin-1-yl)-3-(3-(oxetan-3-yloxy)phenyl)butanoate (Intermediate 41) (50 mg, 0.100 mmol) in THE (8 mL) was added LiOH (12.03 mg, 0.502 mmol) in water (1.6 mL) and the solution was stirred at RT for 18 h to give batch 2. Batches 1 and 2 were combined, concentrated in vacuo and subjected to preparative HPLC purification on an Xbridge C18 column (75 mm×4.6 mm), eluting with 0 to 95% MeCN in 10 mM ammonium bicarbonate solution, flow rate=18 mL/min, and the relevant fractions were concentrated in vacuo to give the title compound (80 mg) as an off white solid: LCMS (System C) RT=0.79 min, ES+ ve m/z 484 (M+H)⁺; ¹H NMR (DMSO-d₆, 400 MHz) 7.19 (t, J=7.8 Hz, 1H), 7.03 (d, J=7.1 Hz, 1H), 6.86 (d, J=7.8 Hz, 1H), 6.68-6.70 (m, 1H), 6.58 (dd, J=8.1, 1.8 Hz, 1H), 6.26-6.31 (m, 2H), 5.26 (quin, J=5.4 Hz, 1H), 4.92 (t, J=6.7 Hz, 2H), 4.53 (ddd, J=6.9, 5.2, 1.5 Hz, 2H), 3.21-3.27 (m, 3H), 3.10-3.19 (m, 1H), 2.63-2.83 (m, 4H), 2.61 (t, J=6.3 Hz, 2H), 2.31-2.44 (m, 4H), 1.81-2.06 (m, 4H), 1.75 ppm (dt, J=11.4, 6.0 Hz, 2H); [α]_{D}²³=+83 (c=1.0, EtOH).

### Example 9: (S)-3-(3,5-Bis(2-methoxyethoxy)phenyl)-4-((S)-3-fluoro-3-(2-(5,6,7,8-tetrahydro-1,8-naphthyridin-2-yl)ethyl)pyrrolidin-1-yl)butanoic acid

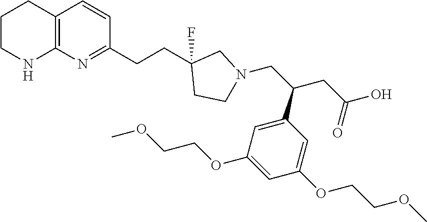 [SMILES: COCCOc1cc(OCCOC)cc([C@H](CC(=O)O)CN2CC[C@@](F)(CCc3ccc4c(n3)NCCC4)C2)c1]

To a solution of (S)-methyl 3-(3,5-bis(2-methoxyethoxy)phenyl)-4-((S)-3-fluoro-3-(2-(5,6,7,8-tetrahydro-1,8-naphthyridin-2-yl)ethyl)pyrrolidin-1-yl)butanoate (Intermediate 44) (400 mg, 0.681 mmol) in THF (4 mL) was added LiOH (3.40 mL, 3.40 mmol) and the reaction mixture stirred for 24 h. The reaction mixture was concentrated in vacuo and subjected to HPLC purification on an Xterra RP C18 (250 mm×19 mm), eluting with 0 to 100% MeCN in 5 mM Ammonium bicarbonate solution, flow rate=18 mL/min, and the relevant fractions were concentrated in vacuo to give the title compound (93 mg, 23%) as a brown gum: LCMS (System C) RT=0.82 min, ES+ ve m/z 560 (M+H)⁺; ¹H NMR (DMSO-d₆, 400 MHz) 8.15 (s, 1H), 7.06 (d, J=7.3 Hz, 1H), 6.41 (d, J=2.0 Hz, 2H), 6.34-6.39 (m, 1H), 6.30 (d, J=7.1 Hz, 1H), 4.05 (dd, J=5.4, 3.9 Hz, 4H), 3.81-3.89 (m, 1H), 3.59-3.67 (m, 4H), 3.31 (s, 6H), 3.21-3.27 (m, 4H), 3.07-3.17 (m, 2H), 2.66-2.87 (m, 4H), 2.61 (t, J=6.2 Hz, 2H), 2.55 (br. s., 1H), 2.41 (dd, J=15.8, 8.5 Hz, 1H), 1.84-2.05 (m, 4H), 1.71-1.79 (m, 2H); Analytical Chiral HPLC on a Chiralpak ID (250 mm×4.6 mm) RT=11.78 min, eluting with 75% ethanol in hexane (containing 0.1% diethylamine), 1 mL/min, detecting at 316 nm.

### Example 10: (3S)-4-((S)-3-Fluoro-3-(2-(5,6,7,8-tetrahydro-1,8-naphthyridin-2-yl)ethyl)pyrrolidin-1-yl)-3-(3-(tetrahydrofuran-3-yl)phenyl)butanoic acid (Isomer 1)

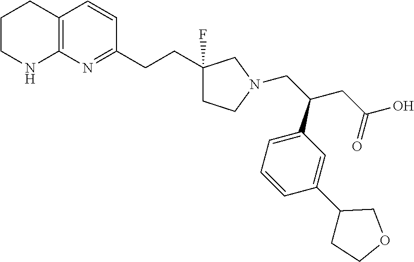 [SMILES: O=C(O)C[C@H](CN1CC[C@@](F)(CCc2ccc3c(n2)NCCC3)C1)c1cccc(C2CCOC2)c1]

A solution of (3,9-Methyl 4-((S)-3-fluoro-3-(2-(5,6,7,8-tetrahydro-1,8-naphthyridin-2-yl)ethyl)pyrrolidin-1-yl)-3-(3-(tetrahydrofuran-3-yl)phenyl)butanoate Isomer 1 (Intermediate 46—Isomer 1) (41 mg, 0.083 mmol), 1M aqueous LiOH solution (0.414 mL, 0.414 mmol) in THF (0.5 mL) was stirred at 25° C. for 18 h. 2M Aqueous HCl solution (0.331 mL, 0.662 mmol) was added and loaded onto a SCX column (5 g), washed with MeCN and eluted with 2M Ammonia in MeOH solution. The relevant fractions were combined and concentrated in vacuo to give the crude compound. The crude compound was subjected to reverse phase column chromatography (4.3 g C18 column) eluting with 15-55% MeCN (containing 0.1% ammonia) in 10 mM aqueous ammonium bicarbonate. The appropriate fractions were combined and concentrated in vacuo to give the title compound (33.8 mg, 85%): LCMS (System A) RT=0.80 min, ES+ ve m/z 482 (M+H)⁺; ¹H NMR (DMSO-d₆, 400 MHz) 7.21 (t, J=7.6 Hz, 1H), 7.17-7.13 (m, 1H), 7.12-7.07 (m, 2H), 7.03 (d, J=7.3 Hz, 1H), 6.32-6.26 (m, 2H), 4.02 (t, J=7.8 Hz, 1H), 3.94 (dt, J=4.5, 8.2 Hz, 1H), 3.79 (q, 8.0 Hz, 1H), 3.53 (t, J=8.1 Hz, 1H), 3.34 (quin, 7.9 Hz, 1H), 3.24 (t, J=4.5 Hz, 2H), 3.21-3.12 (m, 1H), 2.82-2.64 (m, 5H), 2.61 (t, J=6.3 Hz, 2H), 2.57-2.36 (m, 5H), 2.28 (dtd, J=4.5, 7.6, 12.2 Hz, 1H), 2.04-1.81 (m, 6H), 1.79-1.71 (m, 2H).

### Example 11: (3S)-4-((S)-3-Fluoro-3-(2-(5,6,7,8-tetrahydro-1,8-naphthyridin-2-yl)ethyl)pyrrolidin-1-yl)-3-(3-(tetrahydrofuran-3-yl)phenyl)butanoic acid (Isomer 2)

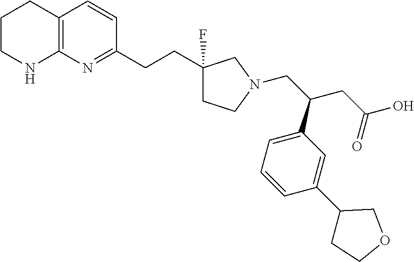 [SMILES: O=C(O)C[C@H](CN1CC[C@@](F)(CCc2ccc3c(n2)NCCC3)C1)c1cccc(C2CCOC2)c1]

A solution of (3,9-Methyl 4-((S)-3-fluoro-3-(2-(5,6,7,8-tetrahydro-1,8-naphthyridin-2-yl)ethyl)pyrrolidin-1-yl)-3-(3-(tetrahydrofuran-3-yl)phenyl)butanoate Isomer 2 (Intermediate 46—Isomer 2) (45 mg, 0.091 mmol), 1M aqueous LiOH solution (0.454 mL, 0.454 mmol) in THE (0.5 mL) was stirred at 25° C. for 18 h. 2M Aqueous HCl solution (0.363 mL, 0.726 mmol) was added and loaded onto a SCX column (5 g), washed with MeCN and eluted with 2M ammonia in MeOH solution. The relevant fractions were combined and concentrated in vacuo to give the crude compound. The crude compound was subjected to reverse phase column chromatography (4.3 g C18 column) eluting with 15-55% MeCN (containing 0.1% ammonia) in 10 mM aqueous ammonium bicarbonate. The appropriate fractions were combined and concentrated in vacuo to give the title compound (41 mg, 93%): LCMS (System A) RT=0.78 min, ES+ ve m/z 482 (M+H)⁺; ¹H NMR (DMSO-d₆, 400 MHz) 7.21 (t, J=7.6 Hz, 1H), 7.17-7.13 (m, 1H), 7.12-7.07 (m, 2H), 7.03 (d, J=7.3 Hz, 1H), 6.32-6.26 (m, 2H), 4.02 (t, J=7.8 Hz, 1H), 3.94 (dt, J=4.5, 8.2 Hz, 1H), 3.79 (q, 8.0 Hz, 1H), 3.53 (t, J=8.1 Hz, 1H), 3.34 (quin, 7.9 Hz, 1H), 3.24 (t, J=4.5 Hz, 2H), 3.21-3.12 (m, 1H), 2.82-2.64 (m, 5H), 2.61 (t, J=6.3 Hz, 2H), 2.57-2.36 (m, 5H), 2.28 (dtd, J=4.5, 7.6, 12.2 Hz, 1H), 2.04-1.81 (m, 6H), 1.79-1.71 (m, 2H).

### Example 12: (S)-4-((S)-3-Fluoro-3-(2-(5,6,7,8-tetrahydro-1,8-naphthyridin-2-yl)ethyl)pyrrolidin-1-yl)-3-(3-(oxetan-3-ylmethoxy)phenyl)butanoic acid

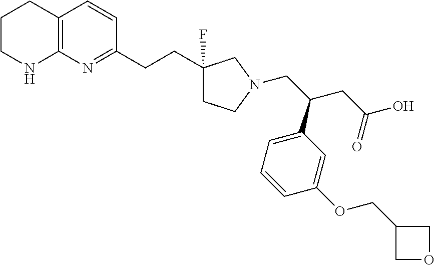 [SMILES: O=C(O)C[C@H](CN1CC[C@@](F)(CCc2ccc3c(n2)NCCC3)C1)c1cccc(OCC2COC2)c1]

To a stirred solution of (S)-methyl 4-((S)-3-fluoro-3-(2-(5,6,7,8-tetrahydro-1,8-naphthyridin-2-yl)ethyl)pyrrolidin-1-yl)-3-(3-(oxetan-3-ylmethoxy)phenyl)butanoate (Intermediate 50) (400 mg, 0.782 mmol) in THF (8 mL) was added a solution of LiOH (94 mg, 3.91 mmol) in water (8 mL) and stirred at ambient temperature overnight. Separately, a solution of (S)-methyl 4-((S)-3-fluoro-3-(2-(5,6,7,8-tetrahydro-1,8-naphthyridin-2-yl)ethyl)pyrrolidin-1-yl)-3-(3-(oxetan-3-ylmethoxy)phenyl)butanoate (Intermediate 50) (100 mg, 0.195 mmol) in THF (2 mL) was added a solution of LiOH (23.40 mg, 0.977 mmol) in water (1.6 mL) and was stirred at ambient temperature overnight. The two reaction batches were combined and concentrated in vacuo and subjected to preparative HPLC purification on an Xbridge C18 column (75 mm×4.6 mm), eluting with 0 to 95% MeCN in 10 mM Ammonium bicarbonate solution, flow rate=1 mL/min, and the relevant fractions were concentrated in vacuo to give the title compound (100 mg, 21%): LCMS (System B) RT=0.46 min, ES+ ve m/z 498 (M+; ¹H NMR (DMSO-d₆, 400 MHz) 7.18 (t, J=7.9 Hz, 1H), 7.02 (d, J=7.1 Hz, 1H), 6.87-6.74 (m, 3H), 6.31-6.24 (m, 2H), 4.70 (t, J=6.9 Hz, 2H), 4.41 (t, J=5.9 Hz, 2H), 4.18 (d, J=6.6 Hz, 2H), 3.36 (td, J=7.0, 13.6 Hz, 1H), 3.28-3.20 (m, 2H), 3.19-3.09 (m, 1H), 2.85-2.56 (m, 9H), 2.44-2.35 (m, 1H), 2.04-1.80 (m, 5H), 1.74 (m, 2H). Analytical chiral SFC on a Chiralpak AS-H column (250 mm×4.6 mm) RT=2.65 min, 93.7%, CO₂, 40% co-solvent (0.5% diethylamine in methanol), 3 g/min, 100 Bar, 30° C., detecting at 323 nm.

### Example 13: 4-((S)-3-Fluoro-3-(2-(5,6,7,8-tetrahydro-1,8-naphthyridin-2-yl)ethyl)pyrrolidin-1-yl)-3-(3-(2-fluoroethoxy)-5-(2-methoxyethoxy)phenyl)butanoic acid

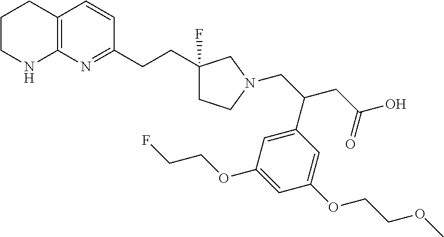 [SMILES: COCCOc1cc(OCCF)cc(C(CC(=O)O)CN2CC[C@@](F)(CCc3ccc4c(n3)NCCC4)C2)c1]

To a stirred solution of methyl 4-((S)-3-fluoro-3-(2-(5,6,7,8-tetrahydro-1,8-naphthyridin-2-yl)ethyl)pyrrolidin-1-yl)-3-(3-(2-fluoroethoxy)-5-(2-methoxyethoxy)phenyl)butanoate (Intermediate 51) (0.2 g, 0.356 mmol) in THF (5 mL) was added a solution of LiOH (8.53 mg, 0.356 mmol) in water (3 mL) dropwise and stirred at ambient temperature for 12 h. The reaction mixture was concentrated in vacuo and co-distilled with MeOH (3×5 mL) to give the crude product. The crude product subjected to preparative HPLC purification on an Xbridge C18 column (75 mm×4.6 mm), eluting with 0 to 100% MeCN in 10 mM Ammonium bicarbonate solution, flow rate=18 mL/min, and the relevant fractions were concentrated in vacuo to give the title compound (55 mg, 28%): LCMS (System B) RT=0.50 min, ES+ ve m/z 548 (M+; ¹H NMR (DMSO-d₆, 400 MHz) 7.02 (d, J=7.3 Hz, 1H), 6.43 (d, J=2.1 Hz, 2H), 6.39-6.34 (m, 1H), 6.28 (d, J=7.2 Hz, 2H), 4.79-4.62 (m, 2H), 4.26-4.12 (m, 2H), 4.08-4.02 (m, 2H), 3.67-3.59 (m, 2H), 3.30 (s, 3H), 3.23 (s, 1H), 3.10 (s, 1H), 2.87-2.63 (m, 5H), 2.60 (s, 3H), 2.55-2.45 (m, 6H), 2.39 (s, 1H), 2.03-1.79 (m, 4H), 1.74 (quin, 5.8 Hz, 2H); Analytical Chiral SFC on a Chiral Pak AD-H column (250 mm×4.6 mm) RT=3.06 min, 73%, CO₂, 40% co-solvent (0.5% diethylamine in methanol), 4 g/min, 100 Bar, 30° C., detecting at 210 nm. Diastereomeric ratio of 74:26 determined from relative integration of peaks on analytical Chiral SFC at 3.06 min (major) and 3.89 min (minor).

### Example 14: 4-((S)-3-Fluoro-3-(2-(5,6,7,8-tetrahydro-1,8-naphthyridin-2-yl)ethyl)pyrrolidin-1-yl)-3-(2-fluoro-5-(2-methoxyethoxy)phenyl)butanoic acid

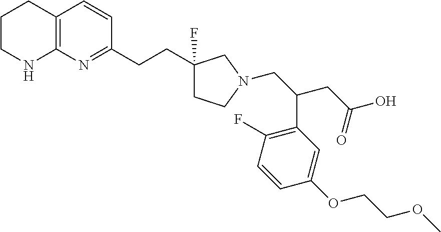 [SMILES: COCCOc1ccc(F)c(C(CC(=O)O)CN2CC[C@@](F)(CCc3ccc4c(n3)NCCC4)C2)c1]

The title compound was prepared by a similar procedure to that described for Example 1 from the corresponding methyl ester (Intermediate 52). Obtained (27 mg, 13%): LCMS (System A) RT=0.80 min, ES+ ve m/z 504 (M+H)⁺; ¹H NMR (400 MHz, D₂O) 7.48 (d, J=7.3 Hz, 1H), 7.15 (t, J=10.1 Hz, 1H), 7.02-6.92 (m, 2H), 6.59 (d, J=7.3 Hz, 1H), 4.25-4.16 (m, 2H), 3.87-3.79 (m, 2H), 3.74-3.32 (m, 8H), 3.46 (s, 3H), 3.31-3.12 (m, 1H), 2.88-2.54 (m, 6H), 2.48-2.30 (m, 1H), 2.30-2.14 (m, 3H), 1.92 (quin, 5.9 Hz, 2H); ¹⁹F NMR (376 MHz, D₂O) −127.07 (0.2 F), −127.14 (0.8 F), −144.22 (1 F). Diastereomeric ratio of 4:1 determined from relative integration of ¹⁹F NMR peaks −127.14 (major) and −127.07 (minor).

### Example 15: (S)-4-((S)-3-Fluoro-3-(2-(5,6,7,8-tetrahydro-1,8-naphthyridin-2-yl)ethyl)pyrrolidin-1-yl)-3-(3-((tetrahydro-2H-pyran-4-yl)oxy)phenyl)butanoic acid

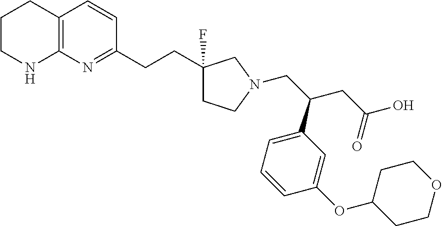 [SMILES: O=C(O)C[C@H](CN1CC[C@@](F)(CCc2ccc3c(n2)NCCC3)C1)c1cccc(OC2CCOCC2)c1]

To a solution of tert-butyl (S)-4-((S)-3-fluoro-3-(2-(5,6,7,8-tetrahydro-1,8-naphthyridin-2-yl)ethyl)pyrrolidin-1-yl)-3-(3-((tetrahydro-2H-pyran-4-yl)oxy)phenyl)butanoate (Intermediate 49—Isomer 1) (480 mg, 0.845 mmol) in 2-MeTHF (5 mL) was added 12M aqueous HCl solution (0.352 mL, 4.23 mmol) and the mixture was stirred at 40° C. under nitrogen for 1 h. The reaction mixture was separated between ethyl acetate and water. The pH of aqueous phase was adjusted to 8 using solid sodium bicarbonate. This was extracted with DCM and passed through a hydrophobic frit and evaporated in vacuo to give a white foam (367 mg) which was dissolved in DMSO/methanol and subjected to reverse phase column chromatography (30 g C18 column) eluting with 5-55% MeCN (containing 0.1% ammonia) in 10 mM aqueous ammonium bicarbonate. The appropriate fractions were combined and the pH adjusted to 8 using solid sodium bicarbonate. This was extracted with DCM and passed through a hydrophobic frit. The solvent was removed in vacuo to give the title compound (229 mg, 53%) as a white foam; LCMS (System B) RT=0.82 min, ES+ ve m/z 512 (M+; ¹H NMR (CDCl₃, 400 MHz) 8.55 (br. s., 1H), 7.24-7.13 (m, 2H), 6.86-6.71 (m, 3H), 6.32 (d, J=7.1 Hz, 1H), 4.48 (tt, J=3.9, 7.8 Hz, 1H), 4.18-4.11 (m, 1H), 4.05-3.94 (m, 2H), 3.59 (ddd, J=3.2, 8.4, 11.6 Hz, 2H), 3.52-3.37 (m, 3H), 3.00-2.39 (m, 10H), 2.24-1.71 (m, 11H).

### Example 16: (S)-4-((S)-3-Fluoro-3-(2-(5,6,7,8-tetrahydro-1,8-naphthyridin-2-yl)ethyl)pyrrolidin-1-yl)-3-(3-(((R)-tetrahydrofuran-3-yl)oxy)phenyl)butanoic acid (1:1) citrate salt

(S)-4-((S)-3-Fluoro-3-(2-(5,6,7,8-tetrahydro-1,8-naphthyridin-2-yl)ethyl)pyrrolidin-1-yl)-3-(3-(((R)-tetrahydrofuran-3-yl)oxy)phenyl)butanoic acid (125 mg, 0.25 mmol) (for preparation see Example 3) was dissolved in MeCN (125 μL) and citric acid was added (0.25 mmol). The mixture was heated to 60° C. for 1 h, then cooled to 5° C. at a rate of 0.1° C./min and held at 5° C. for 16 h. The crystalline solids were isolated by vacuum filtration to yield the crystalline citrate salt.

(S)-4-((S)-3-Fluoro-3-(2-(5,6,7,8-tetrahydro-1,8-naphthyridin-2-yl)ethyl)pyrrolidin-1-yl)-3-(3-(((R)-tetrahydrofuran-3-yl)oxy)phenyl)butanoic acid (308.74 mg, 0.62 mmol) (for preparation see Example 3) was suspended in MeCN (1.8 mL) and citric acid was added (108.3 mg, 0.56 mmol). Seeds of crystals (for a preparation see above) were added. The suspension was heated to 60° C. and stirred for 1 h. Then, the suspension was slowly cooled to 20° C. at a rate of 0.1° C./min and stirred at 20° C. for three days. The suspension was heated to 60° C., stirred for 1 h, slowly cooled to 20° C., stirred for 16 h, heated to 40° C., stirred for 1 h, slowly cooled to 20° C., and stirred for a further 16 h. The solids were isolated by filtration under vacuum and air-dried for 4 h yielding the title compound (269 mg, 65%) as a white solid; LCMS (System B) RT=0.82 min, ES+ ve m/z 498 (M+H)⁺; ¹H NMR (600 MHz, DMSO-d₆) 7.20 (t, J=7.9 Hz, 1H), 7.09 (d, J=7.3 Hz, 1H), 6.83 (br d, J=7.6 Hz, 1H), 6.80 (t, J=1.5 Hz, 1H), 6.75 (dd, J=2.5, 8.2 Hz, 1H), 6.45 (br s, 1H), 6.32 (d, J=7.3 Hz, 1H), 5.03-4.97 (m, 1H), 3.88 (dd, J=4.6, 10.1 Hz, 1H), 3.85-3.80 (m, 1H), 3.78-3.73 (m, 2H), 3.26-3.23 (m, 2H), 3.20-3.13 (m, 1H), 2.94-2.72 (m, 5H), 2.72-2.68 (m, 2H), 2.64-2.58 (m, 6H), 2.58-2.52 (m, 2H), 2.43 (dd, J=8.5, 15.8 Hz, 1H), 2.25-2.16 (m, 1H), 2.05-1.85 (m, 5H), 1.75 (quin, J=6.0 Hz, 2H). Anal. Chiral HPLC RT=22.6 min, 100% on a Chiralpak AD-H column (250 mm×4.6 mm) eluting with 30% EtOH-heptane containing 0.1% isopropylamine, flow rate=1 mL/min, detecting at 235 nm.

### Example 17: (S)-4-((S)-3-Fluoro-3-(2-(5,6,7,8-tetrahydro-1,8-naphthyridin-2-yl)ethyl)pyrrolidin-1-yl)-3-(3-(((R)-tetrahydrofuran-3-yl)oxy)phenyl)butanoic acid (1:1) maleate salt

To a mixture of maleic acid (24.5 mg, 0.211 mmol) and MeCN (0.5 mL) was added a solution of (S)-4-((S)-3-Fluoro-3-(2-(5,6,7,8-tetrahydro-1,8-naphthyridin-2-yl)ethyl)pyrrolidin-1-yl)-3-(3-(((R)-tetrahydrofuran-3-yl)oxy)phenyl)butanoic acid (125 mg, 0.25 mmol) (for preparation see Example 3) (100 mg, 0.201 mmol) in THE (0.5 mL) and stirred at ambient temperature for 1 h, then left to stand in the fridge (approximately 3° C.) for 18 h. The sample was placed in the fridge for a further 3 days. The precipitate was collected by filtration, washed with diisopropylether and placed in a vacuum oven at 35° C. for 1 h to give the title compound (96 mg, 78%) as a white solid; LCMS (System B) RT=0.79 min, ES+ ve m/z 498 (M+H)⁺; ¹H NMR (600 MHz, DMSO-d₆) 7.29 (br d, J=7.2 Hz, 1H), 7.24 (t, J=7.9 Hz, 1H), 6.89 (d, J=7.8 Hz, 1H), 6.87 (br s, 1H), 6.91 (br s, 1H), 6.79 (dd, J=2.2, 8.2 Hz, 1H), 6.45 (d, J=7.3 Hz, 1H), 6.05 (s, 2H), 5.04-4.97 (m, 1H), 3.89 (br dd, J=4.6, 10.1 Hz, 1H), 3.83 (q, J=7.8 Hz, 1H), 3.79-3.73 (m, 2H), 3.38-2.90 (m, 9H), 2.75 (dd, J=6.0, 16.1 Hz, 1H), 2.70-2.60 (m, 4H), 2.50-2.46 (m, 1H), 2.26-2.19 (m, 1H), 2.17-1.98 (m, 4H), 1.98-1.91 (m, 1H), 1.78 (quin, J=6.0 Hz, 2H)

### Example 18: (S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoic acid (1:1) citrate salt

(S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoic acid (125 mg, 0.25 mmol) (for preparation see Example 4) was dissolved in MeCN (125 μL) and citric acid was added (0.25 mmol). The mixture was heated to 60° C. for 1 h, then cooled to 5° C. at a rate of 0.1° C./min and held at 5° C. for 16 h. The crystalline solids were isolated by vacuum filtration to yield the crystalline citrate salt.

(S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoic acid (311.6 mg, 0.63 mmol) (for preparation see Example 4) was suspended in MeCN (2.7 mL) and citric acid was added (108.3 mg, 0.56 mmol). Seeds of crystals (for a preparation see above) were added. The suspension was heated to 60° C. and stirred for 1 h. Then, the suspension was slowly cooled to 20° C. at a rate of 0.1° C./min and stirred at 20° C. for three days. The suspension was heated to 60° C., stirred for 1 h, slowly cooled to 20° C., stirred for 16 h, heated to 40° C., stirred for 1 h, slowly cooled to 20° C., and stirred for a further 16 h. The solids were isolated by filtration under vacuum and air-dried for 4 h yielding the title compound (344 mg, 65%) as a white solid; LCMS (System B) RT=0.82 min, ES+ ve m/z 498 (M+H)⁺; ¹H NMR (600 MHz, DMSO-d₆) 7.20 (t, J=7.9 Hz, 1H), 7.09 (d, J=7.3 Hz, 1H), 6.83 (br d, J=7.6 Hz, 1H), 6.80 (t, J=1.5 Hz, 1H), 6.75 (dd, J=2.5, 8.2 Hz, 1H), 6.45 (br s, 1H), 6.32 (d, J=7.3 Hz, 1H), 5.03-4.97 (m, 1H), 3.88 (dd, J=4.6, 10.1 Hz, 1H), 3.85-3.80 (m, 1H), 3.78-3.73 (m, 2H), 3.26-3.23 (m, 2H), 3.20-3.13 (m, 1H), 2.94-2.72 (m, 5H), 2.72-2.68 (m, 2H), 2.64-2.58 (m, 6H), 2.58-2.52 (m, 2H), 2.43 (dd, J=8.5, 15.8 Hz, 1H), 2.25-2.16 (m, 1H), 2.05-1.85 (m, 5H), 1.75 (quin, J=6.0 Hz, 2H). Anal. Chiral HPLC RT=26.1 min, 100% on a Chiralpak AD-H column (250 mm×4.6 mm) eluting with 30% EtOH-heptane containing 0.1% isopropylamine, flow rate=1 mL/min, detecting at 235 nm.

### Example 19: (S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoic acid (1:1) maleate salt

To a mixture of maleic acid (24.5 mg, 0.211 mmol) and MeCN (0.5 mL) was added a solution of (S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoic acid (125 mg, 0.25 mmol) (for preparation see Example 4) (100 mg, 0.201 mmol) in THE (0.5 mL) and stirred at ambient temperature for 3 h, then left to stand in the fridge (approximately 3° C.) for 18 h. The mixture was removed from the fridge and was added diisopropylether dropwise until a precipitate remained. The sample was placed in the fridge for a further 3 days. The mixture was removed from the fridge, diluted with diisopyl ether (5 mL) and stirred for 1 h and the resulting solid was collected by filtration, washed with diisopropyl ether and placed in a vacuum oven at 35° C. for 1 h to give the title compound (94 mg, 76%) as a white solid; LCMS (System B) RT=0.79 min, ES+ ve m/z 498 (M+H)⁺; ¹H NMR (600 MHz, DMSO-d₆) 7.31 (br d, J=7.1 Hz, 1H), 7.24 (t, J=7.9 Hz, 1H), 6.95 (br s, 1H), 6.89 (d, J=7.8 Hz, 1H), 6.87 (br s, 1H), 6.80 (dd, J=2.2, 8.2 Hz, 1H), 6.46 (d, J=7.2 Hz, 1H), 6.05 (s, 2H), 5.04-4.97 (m, 1H), 3.89 (dd, J=4.6, 10.1 Hz, 1H), 3.83 (q, J=7.8 Hz, 1H), 3.79-3.73 (m, 2H), 3.38-2.90 (m, 9H), 2.75 (dd, J=6.0, 16.1 Hz, 1H), 2.70-2.60 (m, 4H), 2.51-2.47 (m, 1H), 2.26-2.19 (m, 1H), 2.17-1.98 (m, 4H), 1.98-1.91 (m, 1H), 1.78 (quin, J=6.0 Hz, 2H).

### Biological Assays

### Cell Adhesion Assays

Reagents and methods utilised are as described [Ludbrook et al, Biochem. J. 2003, 369, 311 and Macdonald et al. ACS Med. Chem. Lett. 2014, 5, 1207-1212 for αᵥβ₈ assay), with the following points of clarification. The following cell lines are used, with ligands in brackets: K562-αᵥβ₃ (LAP-b₁), K562-αᵥβ5 (Vitronectin), K562-αᵥβ₆ (LAP-b₁), K562-αᵥβ₈ (LAP-b₁), A549-αᵥβ₁ (LAP-b₁). The divalent cation used to facilitate adhesion is 2 mM MgCl₂. Adhesion is quantified by cell labelling with the fluorescent dye BCECF-AM (Life Technologies), where cell suspensions at 3×10⁶ cells/mL are incubated with 0.33 uL/mL of 30 mM BCECF-AM at 37° C. for 10 minutes, then 50 μL/well are dispensed into the 96-well assay plate. At the assay conclusion cells that adhered are lysed using 50 μL/well of 0.5% Triton X-100 in H₂O to release fluorescence. Fluorescence intensity is detected using an Envision® plate reader (Perkin Elmer). For active antagonists in the assay, data is fitted to a 4 parameter logistic equation for IC₅₀ determinations.

All of the exemplified compounds were generally tested according to the above assays and were found to be αᵥβ₆ integrin antagonists. Those of skill in the art will recognise that in vitro binding assays and cell-based assays for functional activity are subject to experimental variability. Accordingly, it is to be understood that the values given below are exemplary only and that repeating the assay run(s) may result in somewhat different pIC₅₀ values.

The mean affinities (pIC₅₀) for Example 1 in the cell Adhesion Assays was for: αᵥβ₆ pIC₅₀=7.6; αᵥβ₁ pIC₅₀=5.7; αᵥβ₃ pIC₅₀=7.1; αᵥβ₅ pIC₅₀=6.6; αᵥβ₈ pIC₅₀=7.0.

The mean affinities (pIC₅₀) for Example 2 in the cell Adhesion Assays was for: αᵥβ₆ pIC₅₀=7.8; αᵥβ₁ pIC₅₀=6.0; αᵥβ₃ pIC₅₀=7.2; αᵥβ₅ pIC₅₀=7.0; αᵥβ₈ pIC₅₀=7.0.

The mean affinities (pIC₅₀) for Example 3 in the cell Adhesion Assays was for: αᵥβ₆ pIC₅₀=8.3; αᵥβ₁ pIC₅₀=6.7; αᵥβ₃ pIC₅₀=7.0; αᵥβ₅ pIC₅₀=7.4; αᵥβ₈ pIC₅₀=7.3.

The mean affinities (pIC₅₀) for Example 4 in the cell Adhesion Assays was for: αᵥβ₆ pIC₅₀=8.3; αᵥβ₁ pIC₅₀=7.0; αᵥβ₃ pIC₅₀=7.3; αᵥβ₅ pIC₅₀=7.1; αᵥβ₈ pIC₅₀=7.5.

The mean affinities (pIC₅₀) for Example 5 in the cell Adhesion Assays was for: αᵥβ₆ pIC₅₀=7.9; αᵥβ₁ pIC₅₀=6.7; αᵥβ₃ pIC₅₀=7.5; αᵥβ₅ pIC₅₀=7.6; αᵥβ₈ pIC₅₀=7.5.

The mean affinities (pIC₅₀) for Example 6 in the cell Adhesion Assays was for: αᵥβ₆ pIC₅₀=7.9; αᵥβ₁ pIC₅₀=6.9; αᵥβ₃ pIC₅₀=7.2; αᵥβ₅ pIC₅₀=6.5; αᵥβ₈ pIC₅₀=7.4.

The mean affinities (pIC₅₀) for Example 7 in the cell Adhesion Assays was for: αᵥβ₆ pIC₅₀=7.7; αᵥβ₁ pIC₅₀=7.2; αᵥβ₃ pIC₅₀=7.1; αᵥβ₅ pIC₅₀=7.2; αᵥβ₈ pIC₅₀=7.3.

The mean affinities (pIC₅₀) for Example 8 in the cell Adhesion Assays was for: αᵥβ₆ pIC₅₀=7.9; αᵥβ₁ pIC₅₀=6.4; αᵥβ₃ pIC₅₀=7.0; αᵥβ₅ pIC₅₀=7.2; αᵥβ₈ pIC₅₀=7.5.

The mean affinities (pIC₅₀) for Example 9 in the cell Adhesion Assays was for: αᵥβ₆ pIC₅₀=8.0; αᵥβ₁ pIC₅₀=6.0; αᵥβ₃ pIC₅₀=7.4; αᵥβ₅ pIC₅₀=ND (not determined); αᵥβ₈ pIC₅₀=7.3.

The mean affinities (pIC₅₀) for Example 10 in the cell Adhesion Assays was for: αᵥβ₆ pIC₅₀=7.9; αᵥβ₁ pIC₅₀=6.4; αᵥβ₃ pIC₅₀=7.2; αᵥβ₅ pIC₅₀=7.1; αᵥβ₈ pIC₅₀=7.7.

The mean affinities (pIC₅₀) for Example 11 in the cell Adhesion Assays was for: αᵥβ₆ pIC₅₀=7.9; αᵥβ₁ pIC₅₀=6.9; αᵥβ₃ pIC₅₀=7.3; αᵥβ₅ pIC₅₀=6.9; αᵥβ₈ pIC₅₀=7.7.

The mean affinities (pIC₅₀) for Example 12 in the cell Adhesion Assays was for: αᵥβ₆ pIC₅₀=7.8; αᵥβ₁ pIC₅₀=6.6; αᵥβ₃ pIC₅₀=7.2; αᵥβ₅ pIC₅₀=7.5; αᵥβ₈ pIC₅₀=7.6.

The mean affinities (pIC₅₀) for Example 13 in the cell Adhesion Assays was for: αᵥβ₆ pIC₅₀=8.0; αᵥβ₁ pIC₅₀=6.9; αᵥβ₃ pIC₅₀=7.1; αᵥβ₅ pIC₅₀=7.0; αᵥβ₈ pIC₅₀=7.5.

The mean affinities (pIC₅₀) for Example 14 in the cell Adhesion Assays was for: αᵥβ₆ pIC₅₀=7.6; αᵥβ₁ pIC₅₀=5.7; αᵥβ₃ pIC₅₀=6.3; αᵥβ₅ pIC₅₀=7.6; αᵥβ₈ pIC₅₀=6.8.

The mean affinities (pIC₅₀) for Example 15 in the cell Adhesion Assays was for: αᵥβ₆ pIC₅₀=8.0; αᵥβ₁ pIC₅₀=6.5; αᵥβ₃ pIC₅₀=7.7; αᵥβ₅ pIC₅₀=7.4; αᵥβ₆ pIC₅₀=7.9.

The mean affinities (pIC₅₀) for Example 16 in the cell Adhesion Assays was for: αᵥβ₆ pIC₅₀=8.3; αᵥβ₁ pIC₅₀=6.8; αᵥβ₃ pIC₅₀=7.6; αᵥβ₅ pIC₅₀=7.4; αᵥβ₆ pIC₅₀=7.9.

The mean affinities (pIC₅₀) for Example 17 in the cell Adhesion Assays was for: αᵥβ₆ pIC₅₀=8.2; αᵥβ₁ pIC₅₀=6.9; αᵥβ₃ pIC₅₀=7.3; αᵥβ₅ pIC₅₀=8.1; αᵥβ₈ pIC₅₀=7.7.

The mean affinities (pIC₅₀) for Example 18 in the cell Adhesion Assays was for: αᵥβ₆ pIC₅₀=8.3; αᵥβ₁ pIC₅₀=6.4; αᵥβ₃ pIC₅₀=7.3; αᵥβ₅ pIC₅₀=7.5; αᵥβ₈ pIC₅₀=7.7.

The mean affinities (pIC₅₀) for Example 19 in the cell Adhesion Assays was for: αᵥβ₆ pIC₅₀=8.2; αᵥβ₁ pIC₅₀=6.6; αᵥβ₃ pIC₅₀=7.3; αᵥβ₅ pIC₅₀=7.5; αᵥβ₈ pIC₅₀=7.4.

1-32. (canceled)

33. A compound of Formula (I) or a pharmaceutically acceptable salt thereof: 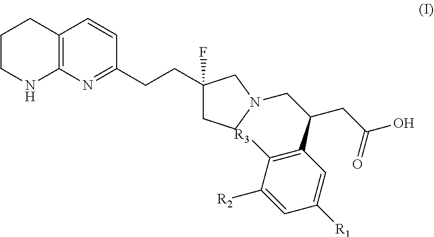
wherein:
R₁ and R₂ are each independently hydrogen or —O—CR₅R₆—CR₇R₈—O(C₁₋₂-alkyl),
wherein R₁ and R₂ are not both hydrogen;
or R₂ is hydrogen and R₁ is
(i) a group selected from 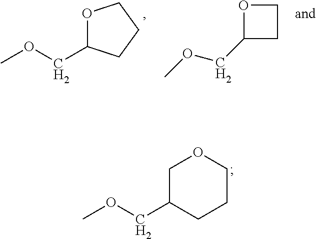
or
(ii) a group selected from 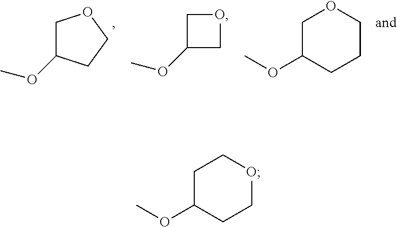
or
(iii) a group selected from 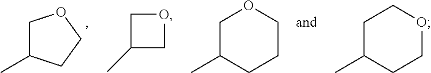
or R₂ is hydrogen and R₁ is 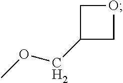
or one of R₁ and R₂ is —O(CH₂)₂OMe and the other is —O(CH₂)₂F;
R₃ is hydrogen or fluoro;
wherein R₃ is hydrogen when R₁ and R₂ are not hydrogen; and
R₅, R₆, R₇ and R₈ are each independently hydrogen or methyl;
wherein said compound is not (S)-4-((S)-3-fluoro-3-(2-(5,6,7,8-tetrahydro-1,8-naphthyridin-2-yl)ethyl)pyrrolidin-1-yl)-3-(3-(2-methoxyethoxy)phenyl)butanoic acid.

34. The compound or pharmaceutically acceptable salt thereof according to claim 33,
wherein:
R₁ and R₂ are each independently hydrogen or —O—CR₅R₆—CR₇R₈—O(C₁₋₂-alkyl),
wherein R₁ and R₂ are not both hydrogen;
or R₂ is hydrogen and R₁ is
(i) a group selected from 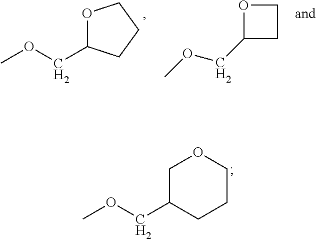
or
(ii) a group selected from 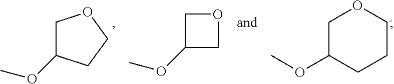
or
(iii) a group selected from 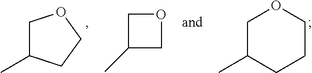 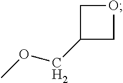
or R₂ is hydrogen and R₁ is
or one of R₁ and R₂ is —O(CH₂)₂OMe and the other is —O(CH₂)₂F;
R₃ is hydrogen or fluoro,
wherein R₃ is hydrogen when R₁ and R₂ are not hydrogen; and
R₅, R₆, R₇ and R₈ are each independently hydrogen or methyl.

35. The compound or pharmaceutically acceptable salt thereof according to claim 33, wherein one of R₁ and R₂ is hydrogen and the other is —O—CRR₆—CR₇R₈—O(C₁₋₂-alkyl), and R₅, R₆, R₇ and R₈ are each independently hydrogen or methyl.

36. The compound or pharmaceutically acceptable salt thereof according to claim 35, wherein one of R₁ and R₂ is hydrogen and the other is selected from 2-methoxyethoxy, 2-methoxypropoxy, 2-methoxy-2-methylpropoxy, (1-methoxypropan-2-yl)oxy, and (1-methoxy-2-methylpropan-2-yl)oxy.

37. The compound or pharmaceutically acceptable salt thereof according to claim 36, wherein one of R₁ and R₂ is hydrogen and the other is selected from 2-methoxypropoxy and (1-methoxy-2-methylpropan-2-yl)oxy.

38. The compound or pharmaceutically acceptable salt thereof according to claim 33, wherein both R₁ and R₂ are —O—CRR₆—CR₇R₈—O(C₁₋₂-alkyl), and R₅, R₆, R₇ and R₈ are each independently hydrogen or methyl.

39. The compound or pharmaceutically acceptable salt thereof according to claim 38, wherein both R₁ and R₂ are 2-methoxyethoxy.

40. The compound or pharmaceutically acceptable salt thereof according to claim 33, wherein R₂ is hydrogen and R₁ is (tetrahydrofuran-2-yl)methoxy.

41. The compound or pharmaceutically acceptable salt thereof according to claim 33, wherein R₂ is hydrogen and R₁ is (tetrahydrofuran-3-yl)oxy.

42. The compound or pharmaceutically acceptable salt thereof according to claim 33, wherein R₂ is hydrogen and R₁ is tetrahydrofuran-3-yl.

43. The compound or pharmaceutically acceptable salt thereof according to claim 33, wherein R₂ is hydrogen and R₁ is (tetrahydropyran-4-yl)-oxy.

44. The compound or pharmaceutically acceptable salt thereof according to claim 33, wherein R₂ is hydrogen and R₁ is oxetan-3-yloxy.

45. The compound or pharmaceutically acceptable salt thereof according to claim 33, wherein R₃ is hydrogen.

46. The compound or pharmaceutically acceptable salt thereof according to claim 33, wherein R₃ is fluoro.

47. The compound or pharmaceutically acceptable salt thereof according to claim 46, wherein R₂ represents hydrogen.

48. The compound or pharmaceutically acceptable salt thereof according to claim 33, which is:
(S)-4-((S)-3-Fluoro-3-(2-(5,6,7,8-tetrahydro-1,8-naphthyridin-2-yl)ethyl)pyrrolidin-1-yl)-3-(3-(((R)-tetrahydrofuran-2-yl)methoxy)phenyl)butanoic acid;
(S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-2-yl)methoxy)phenyl)butanoic acid;
(S)-4-((S)-3-Fluoro-3-(2-(5,6,7,8-tetrahydro-1,8-naphthyridin-2-yl)ethyl)pyrrolidin-1-yl)-3-(3-(((R)-tetrahydrofuran-3-yl)oxy)phenyl)butanoic acid;
(S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoic acid;
((S)-4-((S)-3-Fluoro-3-(2-(5,6,7,8-tetrahydro-1,8-naphthyridin-2-yl)ethyl)pyrrolidin-1-yl)-3-(3-((R)-2-methoxypropoxy)phenyl)butanoic acid;
((S)-4-((S)-3-Fluoro-3-(2-(5,6,7,8-tetrahydro-1,8-naphthyridin-2-yl)ethyl)pyrrolidin-1-yl)-3-(3-((S)-2-methoxypropoxy)phenyl)butanoic acid;
(S)-4-((S)-3-Fluoro-3-(2-(5,6,7,8-tetrahydro-1,8-naphthyridin-2-yl)ethyl)pyrrolidin-1-yl)-3-(3-((I-methoxy-2-methylpropan-2-yl)oxy)phenyl)butanoic acid;
(S)-4-((S)-3-Fluoro-3-(2-(5,6,7,8-tetrahydro-1,8-naphthyridin-2-yl)ethyl)pyrrolidin-1-yl)-3-(3-(oxetan-3-yloxy)phenyl)butanoic acid;
(S)-3-(3,5-Bis(2-methoxyethoxy)phenyl)-4-((S)-3-fluoro-3-(2-(5,6,7,8-tetrahydro-1,8-naphthyridin-2-yl)ethyl)pyrrolidin-1-yl)butanoic acid;
(3S)-4-((S)-3-Fluoro-3-(2-(5,6,7,8-tetrahydro-1,8-naphthyridin-2-yl)ethyl)pyrrolidin-1-yl)-3-(3-(tetrahydrofuran-3-yl)phenyl)butanoic acid (Isomer 1);
(3S)-4-((S)-3-Fluoro-3-(2-(5,6,7,8-tetrahydro-1,8-naphthyridin-2-yl)ethyl)pyrrolidin-1-yl)-3-(3-(tetrahydrofuran-3-yl)phenyl)butanoic acid (Isomer 2);
(S)-4-((S)-3-Fluoro-3-(2-(5,6,7,8-tetrahydro-1,8-naphthyridin-2-yl)ethyl)pyrrolidin-1-yl)-3-(3-(oxetan-3-ylmethoxy)phenyl)butanoic acid;
4-((S)-3-Fluoro-3-(2-(5,6,7,8-tetrahydro-1,8-naphthyridin-2-yl)ethyl)pyrrolidin-1-yl)-3-(3-(2-fluoroethoxy)-5-(2-methoxyethoxy)phenyl)butanoic acid; or
4-((S)-3-Fluoro-3-(2-(5,6,7,8-tetrahydro-1,8-naphthyridin-2-yl)ethyl)pyrrolidin-1-yl)-3-(2-fluoro-5-(2-methoxyethoxy)phenyl)butanoic acid;
or a pharmaceutically acceptable salt thereof.

49. The compound or pharmaceutically acceptable salt thereof according to claim 33, which is (S)-4-((S)-3-Fluoro-3-(2-(5,6,7,8-tetrahydro-1,8-naphthyridin-2-yl)ethyl)pyrrolidin-1-yl)-3-(3-((tetrahydro-2H-pyran-4-yl)oxy)phenyl)butanoic acid.

50. The compound or pharmaceutically acceptable salt thereof according to claim 33, which is (S)-4-((S)-3-Fluoro-3-(2-(5,6,7,8-tetrahydro-1,8-naphthyridin-2-yl)ethyl)pyrrolidin-1-yl)-3-(3-(((R)-tetrahydrofuran-3-yl)oxy)phenyl)butanoic acid.

51. A pharmaceutically acceptable salt of the compound according to 50, wherein the salt is a maleate or a citrate.

52. The compound or pharmaceutically acceptable salt thereof according to claim 33, which is (S)-4-((S)-3-Fluoro-3-(2-(5,6,7,8-tetrahydro-1,8-naphthyridin-2-yl)ethyl)pyrrolidin-1-yl)-3-(3-(((S)-tetrahydrofuran-3-yl)oxy)phenyl)butanoic acid.

53. A pharmaceutically acceptable salt of the compound according to claim 52, wherein the salt is a maleate or a citrate.

54. A pharmaceutical composition comprising the compound or pharmaceutically acceptable salt thereof according to claim 33 and a pharmaceutically acceptable carrier, diluent, or excipient.

55. A method of treating a disease or condition in a human, wherein the disease or condition is responsive to antagonism of an αᵥβ₆ receptor, the method comprising administering to the human in need thereof a therapeutically effective amount of a compound or pharmaceutically acceptable salt thereof according to claim 33.

56. The method according to claim 55, wherein the disease or condition is a fibrotic disease.

57. The method according to claim 56, wherein the fibrotic disease is idiopathic pulmonary fibrosis.
